(19) United States
(12) Patent Application Publication
(10) Pub. No.: US 2010/0092562 A1
(43) Pub. Date: Apr. 15, 2010

(54) SUSTAINED-RELEASE DRUG DELIVERY COMPOSITIONS AND METHODS

(76) Inventors: R. Gary Hollenbeck, Ellicott City, MD (US); James Joseph McDermott, Glen Allen, VA (US); Craig Linwood Attkisson, Richmond, VA (US)

Correspondence Address: JONES DAY, 222 EAST 41ST ST, NEW YORK, NY, 10017, US

(21) Appl. No.: 12/434,413

(22) Filed: May 1, 2009

Related U.S. Application Data
(63) Continuation-in-part of application No. 11/198,937, filed on Aug. 4, 2005.
Application No. 11/198,937 is a continuation of application No. 11/150,572, filed on Jun. 9, 2005.
Application No. 11/150,572 is a continuation-in-part of application No. 10/724,276, filed on Nov. 26, 2003, now abandoned.
(60) Provisional application No. 60/429,202, filed on Nov. 26, 2002.

(51) Int. Cl. A61K 9/16; A61P 11/00; A61P 37/08

(52) U.S. Cl. 424/488; 424/484; 424/490

(57) ABSTRACT

The present invention relates to liquid sustained release suspension dosage forms. In particular, the invention encompasses sustained release compositions comprising a dispersed phase, which contains an ion-exchange matrix drug complex, a diffusion controlling membrane coating and a dispersion medium comprising an excipient capable of impeding water activity such that drug dissolution is inhibited prior to administration. Further, the invention provides for compositions wherein several active ingredients associate in a single bead in the dispersed phase, such that the abuse potential of such active ingredients is reduced. The invention also encompasses sustained release formulations of combination drugs comprising an extended release phase and an immediate release phase. The formulations of the invention may be used to treat a variety of conditions and symptoms, including those that require administration of several drugs, such as cold and allergy symptoms. In one of the embodiments, the sustained release composition combines an antihistamine, an antitussive and a decongestant. The invention further provides for methods of making and using such formulations.

This application is a continuation-in-part of U.S. patent application Ser. No. 11/198,937, filed Aug. 4, 2005, which is a continuation of U.S. patent application Ser. No. 11/150,572, filed Jun. 9, 2005, which is a continuation-in-part of U.S. patent application Ser. No. 10/724,276, filed Nov. 26, 2003, which is entitled to and claims priority benefit under 35 U.S.C. §119(e) to U.S. Provisional Patent Application No. 60/429,202, filed Nov. 26, 2002, each of which is incorporated herein by reference in its entirety.

### 1. FIELD OF THE INVENTION

The present invention relates to liquid sustained release suspension dosage forms comprising ionized forms of water-soluble drugs. The present invention also relates to sustained release drug delivery formulations comprising a combination of such drugs in an extended release and immediate release forms.

### 2. BACKGROUND OF THE INVENTION

Liquid formulations have the distinct advantages of dosage flexibility and ease of swallowing. In addition, it is possible to administer, in a single volume of liquid, a relatively large quantity of dispersed solid, which would normally require several tablets or capsules. Moreover, there is a recognized need for sustained release formulations to be available in a convenient, easy-to-take liquid dosage form. However, the formulation of liquid oral suspensions having sustained-released capabilities has only resulted in limited success. In fact, liquid oral suspensions having a 24 hour sustained release profile has been extremely difficult to achieve. In part, this is due to the challenges presented in maintaining the stability of sustained-release particles when present in liquid dispersal systems, and the difficulty in achieving sustained release of the drug from the dispersed phase. In addition, there are challenges associated with maintaining the stability of liquid sustained release compositions in the presence of ionic components in the liquid phase of such compositions. Specifically, it has been difficult to achieve stable sustained release drug formulations in the presence of an additional drug or drugs in the liquid phase, in particular in the presence of ionized or salt forms of drugs in the liquid phase.

Traditionally, the attempts to achieve a liquid oral dosage formulation capable of sustained release have focused on controlling the release of drug from the dispersed phase of a liquid dosage form. Traditional pharmaceutical suspensions have a low concentration of drug in the liquid phase (also called the dispersion medium) and offer a sustained release of drug from the dispersed phase, controlled at least in part, by the rate of the dissolution process.

Fundamental ion-exchange technology has been an approach utilized for achieving sustained release for solid dosage forms and various attempts have been made to further utilize the technology in liquid suspension formulations as well (see, e.g., U.S. Pat. No. 2,990,332 to Keating; Y. Raghunathan et al., J. Pharm. Sci. 70: 379-84 (1981); T. Tarvainen et al., Biomaterials 20:2177-83 (1999); and U.S. Pat. No. 6,247,174 to Horn-ma et al.). However, a recurring problem with traditional ion-exchange resin drug complexes is the rapid rate of counterion exchange, which causes the drug to be released too rapidly or in an uncontrolled manner from the ion-exchange resin.

Diffusion through a porous membrane is one of the classical approaches to achieve sustained release for solid dosage forms, and the Pennkinetic system utilizes a membrane coat to provide diffusion control for the traditional ion-exchange resin drug complex (see, e.g., U.S. Pat. No. 4,996,047 to Kelleher et al.; S. Motycka et al., J. Pharm. Sci. 74:643-46 (1985); M. G. Moldenhauyer et al., J. Pharm. Sci., 79:659-66 (1990); U.S. Pat. No. 5,376,384 to Eichel et al.). However, even though the Pennkinetic ion-exchange system was introduced over 20 years ago, only one product utilizing this technology exists in the market place, possibly due to: the poor suitability of the ion-exchange resin (i.e., hydrophobicity and swelling); complex formulation and manufacturing processes are required; and long term stability problems.

The traditional approach taken to circumvent the problems associated with ion-exchange technology and the application of diffusion membranes or coatings in preparing liquid oral dosage forms is the use of various impregnating or solvating agents (see, e.g., U.S. Pat. No. 4,859,461 to Chow et al.; U.S. Pat. No. 4,221,778 to Raghunathan; EP 171,528; EP 254,811, and EP 254,822; U.S. Pat. No. 5,186,930 to Kogan et al.; U.S. Pat. No. 4,762,709 to Scheumaker). However, there remain challenges in maintaining the physical stability of controlled release suspension dosage forms consisting of coated beads dispersed in an aqueous vehicle. Specifically, because the core bead is “dry”, and the components hydrophilic, diffusion of water across the coating and into the bead produces significant swelling and the development of high osmotic pressure “inside” the bead. This swelling can then lead to the disruption of coatings and certain failure of any control release mechanism reliant on bead coatings.

There also remains a problem of preparing a liquid oral dosage formulations capable of sustained release in the presence of ionic components in the liquid phase of the formulation. Ionic components are known to affect stability of the drug/ion-exchange resin complex and to disturb the expected dissolution profile of the drug from the dispersed phase. The problem was observed to be particularly acute when attempts were made to provide formulations of combination type drugs where each drug was ionic and of the same ionic charge and where one drug was present in the coated, drug-resin complex form while the other was present in its ionic form in the liquid phase (see, e.g., U.S. Pat. No. 4,762,709 to Scheumaker). Specifically, there remains a problem of preparing a stable liquid oral dosage formulations capable of sustained release of one or more drugs from the dispersed solid phase in the presence of ionized forms of drug(s) in the liquid phase.

One solution to the above-described problem was suggested in U.S. Pat. No. 4,762,709 to Scheumaker, by providing that the second drug in the liquid phase, bearing the same ionic charge as the first drug in the dispersed solid phase, is bound to an its own ion-exchange resin. According to the reference, the unbound second drug in the liquid phase of the same ionic charge as the first drug in the dispersed solid phase perturbs the dissolution profile of the first drug unless it is also bound to a resin. A product based on the ion-exchange technology based on this reference is Tussionex® (Hydrocodone Polistirex and Chlorpheniramine Polistirex) Pennkinetic® Extended Release Suspension (Celltech Pharmaceuticals, Inc.), which was recently approved by the U.S. Food and Drug Administration. However, there still remains a need for stable liquid oral dosage formulations capable of sustained release in the presence of unbound ionic components in the liquid phase.

Also, there is a need for formulations containing a combination of drugs. A variety of medical conditions require administration of combination formulations, especially conditions that require administration of several active ingredients and conditions that require chronic administration of drugs. In particular, there is a need for such combination products for treatment of cold symptoms, which otherwise require multiple administrations of single-ingredient, immediate release drugs.

Americans suffer an estimated one billion colds a year, 2 to 4 colds per year for adults and 6 to 10 colds a year for children. National Institute of Allergy and Infectious Diseases (NIAID) Facts Sheets. “The Common Cold,” available at www.niaid.nih.gov/factsheets/cold.htm December, 2004. Approximately nine out of ten Americans will have at least one cold or similar form of upper respiratory infection annually. Colds are the most prevalent illness in children, occurring more frequently then all other diseases combined and accounting for as much as 50% of all school absenteeism. Micromedex Healthcare Series, “The Common Cold Etiology and Treatment,” available at www.thomsonhc.com/hcs/librarian/ND, accessed Jun. 18, 2008. Colds occur most frequently during the colder months spanning from August or early September through March or April. The lower humidity that tends to accompany decreased ambient temperatures may combine with the increased indoor and close quarter person to person interaction to enhance the proliferation of viruses that cause colds. National Institute of Allergy and Infectious Diseases (NIAID) Facts Sheets. “The Common Cold.” available at www.niaid.nih.gov/factsheets/cold.htm, accessed December, 2004.

Patients with the common cold typically present with signs and symptoms of nasal discharge, obstruction of nasal breathing, swelling of the sinus membranes, sneezing, sore throat, cough and headache. Micromedex Healthcare Series, “The Common Cold Etiology and Treatment,” ibid. Seventy to 90% of patients suffer from rhinorrhea or sneezing, 65% of patients have nasal obstruction or congestion or and 25% of patients have cough or hoarseness. Id. Most colds last between 7-14 days. Patients experiencing symptoms longer than 2 weeks may also be affected by allergies. Id.

According to the American Lung Association, colds account for more visits to the doctor than any other condition. American Lung Association, “Cold and Flu Guidelines: The Common Cold,” available at www.lungusa.org, accessed Jun. 16, 2008. In fact, a study on the impact of the common cold showed that in 2003 alone there were more than 100 million physician visits related to colds, at a cost of $7.7 billion. The study also estimated that children missed 189 million days of school annually and parents missed 126 million days of work to care for a child with a cold. WebMD. “Cost of the Common Cold: $40 Billion.” available at www.medmutual.com, accessed Feb. 24, 2003; Fendrick et al. “The Economic Burden of Non-Influenza-Related Viral Respiratory Tract Infection in the United States.” Arch Intern Med. 163(4): 487-494 (2003). When added to the 150 million workdays missed by employees suffering from a cold, the total economic impact of cold-related work loss exceeds $20 billion. Fendrick, ibid.; Garibaldi R A, “Epidemiology of community-acquired respiratory tract infections in adults. Incidence, etiology, and impact”. Am. J. Med. 78 (6B): 32-37 (1985); Common Cold. National Institute of Allergy and Infectious Diseases www3.niaid.nih.gov/healthscience/healthtopics/colds/ Retrieved on Jun. 11, 2008; US Census Bureau. www.Quickfacts.census.gov/qfd/states/00000.html 2004 Estimates.

American spending on over-the-counter (OTC) and prescription drugs for cough and cold relief is in excess of $3 billion annually. WebMD, ibid; Fendrick, ibid. In the USA alone, the common cold leads to 75 to 100 million physician visits annually at a conservative cost estimate of $7.7 billion per year. Americans spend $2.9 billion on over-the-counter drugs and another $400 million on prescription medicines for symptomatic relief. Fendrick., ibid: Garibaldi, ibid. More than one-third of patients who saw a doctor received an antibiotic prescription, which not only contributes to unnecessary costs ($1.1 billion annually on an estimated 41 million antibiotic prescriptions in the United States), but also has implications for antibiotic resistance from overuse of such drugs. Fendrick, ibid. According to IMS Health (August 2007), annual prescriptions for cough, cold and flu medicines were approximately 42,858,000. Because no single active pharmaceutical ingredient (API) treats all cold symptoms, combination products often provide a convenient and sometimes less expensive means of providing relief than the use of multiple single-ingredient products.

Extended release products do already exist on the market that contain one or two of the APIs, as well as some IR products that contain all three drugs. For example, marketed OTC or prescription drugs containing chlorpheniramine (CPM), hydrocodone (HC), and/or pseudoephedrine (PSE) in IR or ER forms include the following:

Products with Pseudoephedrine:

### Afrinol®

### Cenafed®

### D-Isoephedrine

### D-Pseudoephedrine

### Decofed®

### Dimetapp® Decongestant

Dimetapp® Decongestant Pediatric props

### Drixoral® Nasal Decongestant

### Efidac 24® Pseudoephedrine HCl

### Eltor 120®

### Genaphed®

### Isoephedrine

### Maxenal®

### Myfedrine®

### Novafed®

### Pedia Care®

Pseudo 60's®

### Pseudo-12®

### Pseudoephdrine HCl

### Sudafed 12 Hour®

### Sudafed 24 Hour®

### Sudogest

Products with Chlorpheniramine:

### Aller-Chlor

### Antagonate®

### Chlo-Amine®

### Chlor-Trimeton®

### Chlor-Tripolon®

### Dexchlorpheniramine Maleate

### Efidac 24® Chlorpheniramine Maleate

### Gen-Allerate®

### Haynon®

### Histadur®

### Kloromin®

### Mylaramine®

### Novo-Pheniram®

### Phenetron®

### Piriton®

### Polaramine®

### Pyridamal 100®

### Telachlor®

### Teldrin®

Products with Hydrocodone:

Hycodan® (includes homatropine methylbromide)
Lortab® (includes acetaminophen)
Maxidone® (includes acetaminophen)
Norco® (includes acetaminophen)
Vicodin® (includes acetaminophen)
Zydone® (includes acetaminophen)

Products with Pseudoephedrine Chlorpheniramine:

### Allerest®

### Anamine®

### Biohist-LA®

### Brexin®

### Chlordrine® SR

### Chlor-Phed®

Chlor-Trimeton® (4 hour and 12 hour)

### Deconamine®

### De-congestine TR®

### Dynahist ER®

### Histalet® Syrup

### Kronofed-A® Kronocaps

### Kronofed-A-Jr.® Kronocaps

### ND® Clear

### Pseudoephed/Chlorphen 100®

### Rescon®

### Rescon® Jr.

### Rescon® ED

### Ryna®

### Sudafed® Cold and Allergy

### Tanafed®

Products with Chlorpheniramine+Hydrocodone:

### Tussionex® Pennkinetic® Extended Release Suspension

### TussiCaps® Extended Release Capsules

### S-T Forte® 2

Products with Pseudoephedrine+Hydrocodone:

### Detussin® Liquid

### Histussin® D Liquid

### Tyrodone® Liquid

Products with Chlorpheniramine+Pseudoephedrine+Hydrocodone:

### A-G Tussin®

### Atuss® HD

### Cordron-HC®

### Hexatussin®

### Histinex® PV

### Hydrocof-HC®

### Hydron® PCS

### Hydrotuss® HC

### Hyphed®

### KG-Tussin®

### M-End®

### Notuss®

### P-V-Tussin® Syrup

### Pediatex® HC

### Q-V Tussin®

### Tussin-V®

None of the above-mentioned triple-acting formulations (containing CPM, PSE, and HC in a single product) are extended release products for all three drugs. Likewise, no group has established that administration to a patient of a single dose of an oral composition comprising all three active ingredients provides serum levels of the three drugs over 12 hours that are bioequivalent to serum levels achieved upon administration of an appropriate number of doses over 12 hours of FDA-approved immediate release reference listed drug (RLD) compositions comprising the active ingredients.

Similarly, none of the above-mentioned formulations containing hydrocodone and pseudoephedrine (with or without any other active ingredient, such as CMP) are extended release products. No group has established that administration to a patient of a single dose of an oral composition comprising hydrocodone and pseudoephedrine provides serum levels of these two active ingredients over 12 hours that are bioequivalent to serum levels achieved upon administration of an appropriate number of doses over 12 hours of FDA-approved immediate release RLD compositions comprising hydrocodone and/or pseudoephedrine. In fact, no products containing both hydrocodone and pseudoephedrine with or without another active ingredient, immediate or extended release are currently FDA approved. It should be noted that the foregoing list of marketed drugs includes drugs that are not FDA-approved, but are nonetheless marketed.

As relevant to other embodiments of the present invention, diversion and abuse of drugs present in over-the-counter (“OTC”) and prescribed products has escalated in recent years. For example, many OTC cold and allergy tablets contain pseudoephedrine or ephedrine, which is used to clandestinely produce a drug of abuse known as methamphetamine. This drug, also known as “meth,” “speed,” “crank,” or “ice,” is a powerful, addictive stimulant that affects the central nervous system. Methamphetamine is sold illegally in the form of pills, capsules, or powder that can be smoked, snorted, injected, or swallowed.

Makeshift secret and illegal laboratories (“meth labs”) isolate pseudoephedrine or ephedrine from OTC cold and allergy tablets using a solution of water, alcohol, or other solvent for several hours until the pseudoephedrine or ephedrine separates from the tablet. The pseudoephedrine or ephedrine is then be converted into methamphetamine using readily available common household products and easily assessable equipment, such as alcohol, Coleman fuel, acetone, road flares, drain cleaners, iodine, muriatic acid, rock salt, starting fluid, coffee filters and matches. Methods for making the drug are available from “recipes” and exchange of information readily available via the Internet. Methamphetamine trafficking and abuse are on the rise in the United States, and measures to prevent theft and diversion of pseudoephedrine are cumbersome and costly.

Many other OTC and prescription products, containing drugs such as opioids, are also ripe for diversion for illegal drug abuse. For example, hydrocodone is legally used as an antitussive (cough suppressant) in cold medicines, and as analgesic agent for the treatment of moderate to moderately severe pain in prescription medicines. Hydrocodone is the most frequently prescribed opiate in the U.S. with over 110 million prescriptions for hydrocodone-containing products dispensed in 2003. Although it is generally not clandestinely produced, hydrocodone is currently illegally diverted and directly abused for its euphoria and pain-relieving effects. Widespread diversion occurs via bogus call-in prescriptions, altered prescriptions, theft and illicit purchases from Internet sources.

Thus, a need exists for preparing and selling OTC and prescription products that avoid the potential for abuse and diversion of drugs for illegal use. For example, a need exists for cold/cough and allergy formulations where drugs such as pseudoephedrine, ephedrine and/or hydrocodone cannot easily be extracted or separated out in a makeshift laboratory. Likewise, a need exists for cold/cough, pain relief and muscle relaxation products that cannot be easily diverted for illegal purposes, regardless of whether they are clandestinely produced in illegal laboratories.

Citation of any reference in Section 2 of this application is not an admission that the reference is prior art to the application.

### 3. SUMMARY OF THE INVENTION

There remains a need for sustained release liquid dosage forms, in particular dosage forms with better pharmacologic properties and stability and dosage forms which will appeal to the commercial marketplace. In particular, there remains a need for sustained release liquid dosage forms, suitable for administration of drugs fewer times a day, i.e., less often than every 4-6 hours. There remains a need for such sustained release liquid dosage formulations containing a combination of drugs, including drugs in an extended release form and drugs in an immediate release form. In particular, there remains a need for formulations comprising several active ingredients, wherein such formulations can be administered to a patient less often than currently available immediate release product/s, while in the same time achieving bioequivalence to such products. Moreover, there remains a need for achieving sustained release drug delivery formulations that avoid the potential for abuse and diversion of drugs for illegal use.

In one embodiment, the inventors of the present invention have overcome the challenges presented by a drug delivery system that comprises beads containing drug that are coated with a material capable of controlling release of the highly soluble drug and immersed in an aqueous dispersion medium. In particular, the inventors have demonstrated that by manipulating the components of the bead and selection of the suspension medium, initial water penetration into the bead can be precluded and the drug can remain sequestered in the dispersed phase until administration to a patient. Moreover, the inventors have demonstrated that by controlling shifts in the osmotic pressure, the traditional challenges of keeping coatings intact are overcome and controlled release can be readily achieved using polymeric porous or non-porous membranes. In addition, products utilizing the drug delivery systems of the invention have a long shelf life since the drug remains confined in the dispersed phase and any functional coatings remain intact.

As such, the instant invention encompasses liquid sustained release suspension dosage forms that have the following characteristics: capability for maintaining the physical integrity of the coating both in vitro (e.g., in the bottle or in storage) and in vivo for a period long enough to control release; control of water activity such that the water present outside the beads in the product is at an activity less than inside the bead; and the presence of free dissolved drug exists in the reservoir available to diffuse out once the drug is administered. In particular, the invention contemplates the inclusion of a soluble, non-electrolytic component(s) within the bead during manufacture. Such a component will dissolve when water is absorbed into the bead and diffuse out of the reservoir and reduce osmotic pressure, thereby reducing swelling of the bead and loss of integrity of the bead coating. Accordingly, the invention encompasses the use of excipients that are capable of being mass transferred out of the drug-loaded bead when the bead absorbs water, thereby reducing pressure inside the bead and preventing the disruption of bead coatings and facilitating controlled release of the drug.

The inventors have also made the surprising discovery that adding a high concentration of an inactive highly hydrated excipient to the dispersion medium can impede dissolution of the drug in the dispersed phase, and that, therefore, free drug concentration in the dispersion medium is extremely low, or virtually non-existent. As such, the invention also contemplates reducing the water activity in the dispersion medium by adding a high concentration of inactive component that is highly hydrated and capable of associating strongly with water and impeding the association of water with the drug complex of the dispersed phase. As such, water in the dispersion medium is not sufficiently attracted to the drug loaded bead, thereby eliminating dissolution of drug prior to administration and confining the drug in the dispersed phase of a suspension.

Accordingly, in one of the preferred embodiments, the present invention encompasses thermodynamically stable liquid dosage drug suspensions capable of providing sustained drug release when administered to a patient. The present invention encompasses a novel class of sustained release drug formulations comprising a drug confined in a dispersed phase of a suspension through thermodynamically stable electrostatic interactions with a pharmaceutically acceptable ion-exchange matrix having a surface charge opposite that of the drug. Such liquid formulations are capable of achieving sustained release over 12, 24 hours and up to 48 hours. As such, the invention encompasses liquid dosage forms that need only be administered once or twice daily, ensuring ease of administration. It is envisaged by the invention that a soluble non-electrolytic component, having relatively low molecular weight may also be included in the drug complex. In one embodiment, the drug-ion exchange matrix-complex is a bead. It is also envisaged by the invention that the dispersed phase can optionally be membrane-coated with a porous or non-porous polymeric membrane. In one of the embodiments, the dispersion medium also includes a highly hydrated excipient(s) capable of associating closely with water in the dispersion medium, thereby limiting the water activity in the dispersion medium, minimizing water attraction to the drug loaded bead and impeding the dissolution of drug prior to administration. In one embodiment, drug release is activated following administration to a patient. Drug release is triggered when the suspension is placed in an environment, for example gastric or intestinal fluid, with high concentrations of water and small ions that possess the same charge as the drug, as the small ions swamp the diffuse double layer. In certain embodiments, the gastric fluid of the patient dilutes the dispersion medium after administration of the dosage form and the membrane becomes hydrated and more porous, allowing dissolution and dispersion of drug from the beads.

In one embodiment of the invention, the dispersed phase contains more than one drug, e.g., two, three, four or more drugs or active ingredients. In one embodiment, the dispersed phase comprises three drugs or active ingredients. It is also envisaged that two or more drugs associate in a single particulate, pellet or bead. In one of the embodiments, two, three or more drugs associate with the pharmaceutically acceptable ion-exchange matrix having a surface charge opposite that of such drugs. In certain embodiments, two, three or more drugs associate with the same pharmaceutically acceptable ion-exchange matrix having a surface charge opposite that of such drugs in a single particulate, pellet or bead. Surprisingly, inventors found that binding two or more drugs to the same ion-exchange matrix provides an adequate rate of release of the individual drugs and does not interfere with the controlled release. Further, inventors found that binding of two or more drugs to the same ion-exchange matrix ensures dose uniformity of the respective drugs in the combination formulation, and provides an advantage of having only one drug-bound resin complex in the dispersed phase. In yet another embodiment, two or more drugs associate with different pharmaceutically acceptable ion-exchange matrices having a surface charge opposite that of the respective drugs.

In one of the embodiments, the dispersed phase contains a salt form of a drug or active ingredient. In another embodiment, the dispersed phase contains a salt form of the ion-exchange matrix. In one embodiment, the dispersed phase contains pharmaceutically acceptable salt forms of drug/s and/or the ion-exchange matrix.

In one of the embodiments, the drug or drugs in the dispersed phase have a very low rate of release into the dispersion medium before its administration to a patient, i.e., less than 5% based on the total molar amount of drug in the dispersion medium and dispersed phase. The present invention contemplates liquid form controlled release compositions wherein the amount of the drug released from the dispersed phase into the dispersion medium before administration to a patient is less than 30%, less than 25%, less than 20%, less than 15%, less than 10%, less than 5%, less than 0.5%, less than 0.05% based on the total molar amount of drug in the dispersion medium and dispersed phase. In such embodiments, the dispersion medium is physically and chemically stable for more than about one year, more than about 2 years, more than about 3 years, or more than about 4 years. In one embodiment, the dispersion medium may be substantially devoid of free drug. In yet another embodiment, one or more drugs in the dispersed phase is released into the dispersion medium before its administration to a patient, e.g., about 10%, 15%, 20%, 25%, 30%, 35%, 40%, 45% or 50% of the drug is released based on the total molar amount of drug in the dispersion medium and dispersed phase.

In addition, there still remains a need for stable liquid oral dosage formulations capable of sustained release in the presence of unbound ionic components in the liquid phase. In particular, there is a need for formulations containing a combination of drugs, wherein at least one drug is in an extended release form and resides in a dispersed solid phase and wherein another drug is in an immediate release form and resides in the liquid phase. A variety of medical conditions require administration of such combination formulations, especially conditions that require administration of several active ingredients and conditions that require chronic administration of drugs.

Surprisingly, the compositions of the present invention are stable in the presence of ionic components. In one of the embodiments, the composition of the present invention is stable in the presence of ionic components in the dispersion medium, such as electrolytic drugs. Surprisingly, the compositions of the present invention maintain stability in the presence of one or more free drugs in the dispersion medium, i.e., drug or drugs that are not bound to an ion-exchange matrix. Moreover, unexpectedly the compositions of the present invention maintain adequate release of one or more drugs from the dispersed phase in the presence of free drug(s) or ionic component(s) in the dispersion medium. One of the embodiments envisages that the composition comprises one or more drugs in a dispersed phase in an extended release form and one or more drugs in a dispersion medium in an immediate release form. In one such embodiment, drug(s) in the dispersion medium are not bound to an ion-exchange matrix. In another such embodiment, the dispersion medium does not comprise an ion-exchange matrix. In yet another such embodiment, the dispersion medium does not comprise an ion-exchange matrix with the surface charge opposite that of at least one drug of one or more drugs in the dispersion medium. In another embodiment, the dispersion medium contains pharmaceutically acceptable salt form drug(s).

It is envisaged that the compositions of the present invention have a storage shelf life at room temperature conditions of at least 6 months, at least one year, at least about two years, or at least three, four or five years, during which time the stability and drug release profile characteristics of such compositions are maintained.

In one embodiment, the present invention relates to a liquid form controlled release drug composition, comprising:

(a) a dispersed phase comprising an ion-exchange matrix drug complex comprising a pharmaceutically acceptable ion-exchange matrix and at least one water-soluble electrolytic drug associated with the ion-exchange matrix, wherein the surface charge of the ion-exchange matrix is opposite that of the electrolytic drug(s); and

(b) a dispersion medium.

In another embodiment, the present invention relates to a liquid form controlled release drug composition, comprising:

(a) a dispersed phase comprising two or more active ingredients in an extended release form, wherein the dispersed phase comprises particulates, pellets or beads; wherein two or more active ingredients associate in a single particulate, pellet or bead; and

(b) a dispersion medium.

In another embodiment, the present invention relates to a liquid form controlled release drug composition, comprising:

(a) a dispersed phase comprising one or more active ingredients in an extended release form, and

(b) a dispersion medium comprising one or more active ingredients in an immediate release form.

In some embodiments, the present invention relates to a liquid form controlled release drug composition, comprising:

(a) a dispersed phase comprising two or more active ingredients in an extended release form, wherein the dispersed phase comprises particulates, pellets or beads; wherein two or more active ingredients associate in a single particulate, pellet or bead; and

(b) a dispersion medium comprising one or more active ingredients in an immediate release form.

In another embodiment, the dispersed phase further comprises a pharmaceutically acceptable ion-exchange matrix and a water-soluble electrolytic drug(s) associated with the ion-exchange matrix, wherein the surface charge of the ion-exchange matrix is opposite that of the electrolytic drug.

In another embodiment, the dispersed phase is membrane-coated. In particular embodiments, the membrane is polymeric. The membrane can be porous or non-porous. In one preferred embodiment, the membrane is porous. In one such preferred embodiment, the membrane controls diffusion of the drug. In such embodiment, porous membrane coating enables slow release of the drug.

In yet other embodiments, the dispersed phase comprises drug-loaded beads that include a low molecular weight, non-electrolytic soluble excipient(s) capable of dissolving and diffusing out of the beads when water is absorbed into the bead and reducing osmotic pressure inside the beads. In one of the embodiments, the low molecular weight excipient is lactose.

In another embodiment, the dispersion medium further comprises a highly hydrated excipient that attracts water in the dispersion medium. The invention contemplates a high concentration of a highly hydrated excipient, e.g., 50% to 70% on a weight by weight basis of a highly hydrated excipient in the dispersion medium. In one of the embodiments, the highly hydrated excipient in the dispersion medium is sucrose, for example 65% sucrose on a weight by weight basis.

In another embodiment, one or more drugs or active ingredients in a dispersed phase and/or dispersion medium are in a salt form. In yet another embodiment, the dispersed phase comprises a mixture of drug and ion-exchange matrix powders, wherein the drug(s) and the ion-exchange matrix are in a salt form. In one of the embodiments, the drug(s) are a pharmaceutically acceptable salt form of the drug(s).

The present invention also relates to methods for making the liquid form controlled release drug composition. In one embodiment, the invention relates to methods for preparing a liquid form controlled release drug composition, comprising:

(a) allowing a water-soluble electrolytic drug to associate with an ion-exchange matrix to form an ion-exchange matrix drug complex; and

(b) dispersing the ion-exchange matrix drug complex into a dispersion media; wherein

the surface of the ion-exchange matrix has a charge opposite that of the electrolytic drug.

In another embodiment, the invention relates to methods for preparing a liquid form controlled release drug composition, comprising:

(a) preparing the dispersed phase, which comprises preparing particulates, pellets or beads, combining two or more active ingredients so that they associate in a single particulate, pellet or bead, and wherein two or more active ingredients associate in a single particulate, pellet or bead;

(b) coating the particulates, pellets or beads with a membrane coating;

(c) preparing the dispersion medium, wherein the dispersion medium comprises one or more active ingredients; and

(d) dispersing the beads into a dispersion medium.

The present invention also envisages a method of preparing a liquid form controlled release drug compositions, wherein preparing the dispersed phase comprises blending of the two or more active ingredients and ion exchange matrix powders. In one of the embodiments, a dispersed phase is prepared with ionic or salt forms of active ingredients mixed with a salt form of an ion-exchange matrix. In one embodiment the two or more active ingredients comprise an antihistamine (e.g., chlorpheniramine), an antitussive (e.g., hydrocodone), and a decongestant (e.g., pseudoephedrine). In another embodiment, the two or more active ingredients consist of an antihistamine (e.g., chlorpheniramine), an antitussive (e.g., hydrocodone), and a decongestant (e.g., pseudoephedrine). In a certain specific embodiment, the two or more active ingredients consist of chlorpheniramine, hydrocodone and pseudoephedrine.

In a specific embodiment, the salt forms of chlorpheniramine, pseudoephedrine and hydrocodone are allowed to mix with sodium alginate powders in the presence of water and one or more other ingredients. Non-limiting examples of the other ingredients include microcrystalline cellulose, forming drug-loaded beads. In one embodiment, the drug-loaded beads further comprise lactose. In certain embodiments, the resulting beads are coated with EUDRAGIT® in the presence of triethyl citrate and talc, and cured in an oven. The coated beads are suspended in a dispersion medium that comprises salt forms of chlorpheniramine and hydrocodone, water and sucrose. In certain specific embodiments, the dispersion medium comprises Syrup NF. The dispersion medium can also further comprise preservatives and other non-active additives. In such embodiments, the resulting liquid sustained release product is capable of maintaining physical stability in a bottle and capable of achieving controlled release of drug product when administered to a patient.

The present invention also relates to methods for treating a patient suffering from a symptom or condition. In such embodiments, the invention relates to methods for treating a condition or symptom comprising administering a liquid form controlled release drug composition as described herein to a patient in need thereof. The dosage forms of the invention are particularly beneficial to patients who require administration of more than one drug at a time and to patients who require chronic drug administration.

In one specific embodiment, the present invention envisages treatment of cold symptoms using liquid form controlled release drug composition comprising, for example, the following active ingredients: an antihistamine, an antitussive, and a decongestant. In one embodiment, the present invention overcomes problems and disadvantages previously associated with formulations of immediate release (IR) and/or combination products comprising an antihistamine, an antitussive, and a decongestant. The present invention provides unique benefits as compared to immediate release and/or combination cold and allergy products that are current available. For example, in one embodiment, the present invention integrates the benefits of three active pharmaceutical ingredients (“APIs”), i.e., an antihistamine (e.g., chlorpheniramine or “CPM”), an antitussive (e.g., hydrocodone or “HC”), and a decongestant (e.g., pseudoephedrine or “PSE”), into one extended release (ER) (e.g., 12 hour) composition. Previously, when used together, these APIs needed to be dosed 4-6 times daily in their immediate release (IR) forms because they are not currently available in a single extended release triple-acting combination product.

In contrast to the many products currently commercially available, the present invention provides a formulation that allows extended release (ER) (e.g., 12 hour) of all three active ingredients. As one example, the present invention provides an oral formulation that comprises a novel mixture of IR and ER forms of chlorpheniramine (an antihistamine), hydrocodone (a narcotic antitussive) and pseudoephedrine (a decongestant), in a single product. This formulation results in an ER combination product that can be dosed twice daily with the same effectiveness as previously available IR forms (which have been sold and administered either individually or via combination products). The formulation is also superior to existing single and combination ER formulations in that it (a) provides both IR and ER dosages of drugs for immediate and long term drug delivery; (b) provides bioequivalent dosages of three RLDs in a single dosage form; and (c) resists abuse and diversion of the component drugs.

In one embodiment, the present invention provides that administration to a patient of a single dose of an oral drug composition comprising decongestant, antitussive and/or antihistamine as active ingredients provides serum levels of those active ingredients over 12 hours that are bioequivalent to serum levels achieved upon administration of an appropriate number of doses over 12 hours of FDA-approved immediate release reference listed drug (IR RLD) compositions comprising the active ingredients. The appropriate number of doses of the IR RLDs corresponds to the number of doses recommended in one or more FDA-approved labels for the administration of the IR RLDs over 12 hours.

In another embodiment, administration to a patient of a sufficient number of doses of an extended release oral composition comprising decongestant, antitussive and/or antihistamine as active ingredients to achieve steady-state serum levels of the active ingredients over a dosing period of greater than 24 hours yields serum levels of those active ingredients that are bioequivalent to serum levels achieved upon administration of an appropriate number of doses over the same time period of one or more FDA-approved IR drug compositions comprising the active ingredients. The appropriate number of doses of IR drugs corresponds to a number of doses recommended in one or more FDA-approved labels for the administration of the one or more FDA-approved IR drugs over the same time period. In another embodiment, the present invention provides oral formulations comprising hydrocodone and pseudoephedrine, with or without an antihistamine, that allow for extended release of those active ingredients in a human.

In one embodiment, the ER portion of the present invention formulation may exist in the form of coated beads, particulates or pellets within a liquid suspension, with an IR portion in the liquid suspension. Alternatively, the ER portion may comprise a solid dosage form such as capsule, tablet, or other oral solid, with an IR portion as a secondary layer or medium outside the ER portion. In one embodiment, the product is formulated to be dosed once every 12 hours. Other embodiments include those dosed every 8 hours, 16 hours, 24 hours, etc.

Likewise, in another embodiment, a single combination ER product exhibits a specific IR to ER ratio, where the ER component is in a particulate, pellet, or bead and the IR portion is outside the particulates, pellets, or beads (e.g., suspended in syrup; in powder in a capsule, tablet, etc.). The ratio achieves blood serum levels that are bioequivalent (BE) to reference listed drugs (RLDs) at both single-dose and steady state conditions. In other embodiments, one using the present formulations obtains certain specific blood serum ranges (as measured by AUC, Tₘₐₓ, T_{1/2}, etc.) in humans over time, where the levels are bioequivalent to RLDs at both single-dose and steady state conditions.

In one embodiment, the present invention relates to a method for making oral extended release drug compositions comprising a first portion comprising an antihistamine, an antitussive, and optionally a decongestant, as active ingredients in an immediate release form, and a second portion comprising particulates, pellets or beads that comprises the antihistamine, the antitussive and the decongestant as active ingredients in an extended release form. In another embodiment, a method of the present invention involves making a composition so that an IR portion initially comprises an antihistamine and an antitussive, but not a decongestant, and an ER portion comprises an antihistamine, an antitussive and pseudoephedrine. In a related embodiment, leaching of one or more of the ER components, such as pseudoephedrine, into the IR vehicle may be used to provide the IR form of one or more components.

In another embodiment, the present formulation provides particles that comprise all three drugs in a single bead. Such an embodiment offers advantages over previously available ER formulations that provide each drug in separate beads in terms of both dosage efficacy, safety, and resistance to diversion and abuse.

In other embodiments of the present invention, formulations comprise ER particulates, pellets or beads that comprise pseudoephedrine (or chemically related decongestant, such as ephedrine) and/or narcotic antitussives (such as hydrocodone) in a manner that prevents or makes difficult the misuse, abuse or illegal diversion of such drugs, as compared to other commercially available OTC or prescription products comprising one or more of these drugs. For example, if each individual ER particulate, pellet or bead in the present formulations comprises pseudoephedrine (or related compound) along with other compounds, including an antihistamine and antitussive, such as chlorpheniramine and hydrocodone, the formulations will prevent or make difficult the separation or extraction of PSE (or ephedrine) and/or a narcotic antitussive from the final formulation for the purpose of preparing meth or isolating a narcotic.

While it may be technically possible to extract PSE, ephedrine and/or a narcotic from formulations of the present invention (e.g., from the beads), extraction will require elaborate and expensive equipment and techniques. Most illegal producers of meth will not have access to such equipment and/or resources to make extraction worthwhile, especially as compared what can be easily done using other OTC products containing these drugs. Thus, unlike many commercially available OTC formulations containing PSE (or related compound) or a narcotic antitussive, retailers will be able to sell OTC versions of the present formulations that are more freely available.

Thus, embodiments of the present invention will provide unique benefits when compared to immediate release and/or drug combination products that are currently on the market. Such benefits include: (1) dosing two times a day, instead of every four to six hours, which improves patient compliance and that the correct dose of drug is delivered; (2) a liquid suspension offers more flexible dosing options than tablets, which allows for individualized patient treatment and the ability to titrate therapy based on symptoms; (3) reducing the potential for abuse and/or diversion of hydrocodone and/or pseudoephedrine, resulting from the processing of the APIs in the extended release portion of the formulations; and (4) using a unit of use (4 oz) or unit dose (5 ml to 10 ml) that will be easier to track and can also reduce the potential for diversion.

The present invention can be understood more fully by reference to the following detailed drawings, description and illustrative examples, which exemplify non-limiting embodiments of the invention.

### 4. BRIEF DESCRIPTION OF THE DRAWINGS

FIG. 1 shows one embodiment of the invention, wherein in the liquid form controlled drug release comprises a dispersed phase 1 comprising a calcium alginate matrix drug complex that is coated with a porous diffusion-controlling membrane 5.

FIG. 2 shows a process for release of the electrolytic drug from a dispersed phase 1 comprising a calcium alginate matrix drug complex that is coated with a porous diffusion-controlling membrane 5.

FIG. 4 shows Langmuir interaction isotherms at 25° C. resulting from the interaction of propranolol with the ion-exchange matrices sodium alginate (♦), xanthan gum (▪) and gellan gum ().

FIG. 5 shows Scatchard plots at 25° C. resulting from the interaction of propranolol with the ion-exchange matrices sodium alginate (♦), xanthan gum (▪) and gellan gum ().

FIG. 6 shows a method for preparing calcium alginate in the form of beads by controlled addition of an aqueous suspension of sodium alginate to an aqueous solution of CaCl₂.

FIG. 7 shows a multilayered calcium alginate drug precursor that forms a calcium alginate drug complex when contacted with water.

FIG. 8 shows a diagram of a miniature fluid bed coater.

FIG. 9 shows the release profile of albuterol from coated beads. (Vessels 1-3; 1 g samples of 5% Albuterol Alginate/Lactose 35% Eudragit RS30D: Vessels 4-6, 2 g samples of 2.5% Albuterol Carbopol/Lactose 35% Eudragit RS30D.)

FIG. 10 shows the release profile of albuterol in various coated albuterol/alginate/lactose bead suspensions.

FIG. 11 shows the release profile of albuterol in various coated albuterol/carbopol/lactose bead suspensions.

FIG. 12 shows the effect of dispersion medium components on albuterol release from sustained release suspensions. Alginate bead formulation including 20% lactose; binary Eudragit RL/RS (5:95) coating system applied at a 25% coating level with 10% plasticizer.

FIG. 13 shows the effect of lactose level in bead formulation on albuterol release from sustained release suspensions. Alginate bead formulation; binary Eudragit RL/RS (5:95) coating system applied at a 20% coating level with 10% plasticizer.

FIG. 14 shows the effect of coating level on albuterol release from sustained release suspensions. Alginate bead formulation; binary Eudragit RL/RS coating system applied at a 20% coating level with 10% plasticizer.

FIG. 15 shows the release profile of albuterol from various coated beads in suspension released in water.

FIG. 16 shows the difference in albuterol release from various coated beads in suspension released in water as compared to release in typical buffer systems.

FIG. 17 shows the effect of sucrose concentration in the vehicle on the release of albuterol from beads with 20% coating. Legend indicates coating level first, then sucrose concentration second.

FIG. 18(A) shows the release profile of pseudoephedrine from sustained release suspensions of Formulation X.

FIG. 18(B) shows the release profile of pseudoephedrine from coated beads.

FIG. 19(A) shows the release profile of hydrocodone from sustained release suspensions of Formulation X.

FIG. 19(B) shows the release profile of hydrocodone from coated beads.

FIG. 20(A) shows the release profile of chlorpheniramine from sustained release suspensions of Formulation X.

FIG. 20(B) shows the release profile of chlorpheniramine from coated beads.

FIG. 21(A) shows the release profile of pseudophedrine at time=3 weeks for suspensions of the base formulations (n=3, vessels 4, 5, and 6).

FIG. 21(B) shows the release profile of pseudoephedrine (pseudoephedrine hydrochloride) at time=3 weeks for suspensions of the salt formulations (n=3, vessels 1, 2, and 3).

FIG. 22(A) shows the release profile of hydrocodone at time=3 weeks for suspensions of the base formulations (n=3, vessels 4, 5, and 6).

FIG. 22(B) shows the release profile of hydrocodone (hydrocodone bitartrate) at time=3 weeks for suspensions of the salt formulations (n=3, vessels 1, 2, and 3).

FIG. 23(A) shows the release profile chlorpheniramine at time=3 weeks for suspensions of the base formulations (n=3, vessels 4, 5, and 6).

FIG. 23(B) shows the release profile of chlorpheniramine (chlorpheniramine maleate) at time=3 weeks for suspensions of the salt formulations (n=3, vessels 1, 2, and 3).

FIG. 24 shows release profile of hydrocodone at time=0 from a sustained release suspension containing one active ingredient, hydrocodone (10 mg/5 mL), bound to alginic acid.

### 5. DETAILED DESCRIPTION OF THE INVENTION

### 5.1 Definitions

As used herein, the term “patient” includes, but is not limited to any animal such as a bird (e.g., poultry) or a mammal, including humans, domestic and farm animals, and zoo, sports and pet companion animals such as household pet and other domesticated animals such as, but not limited to, cattle, sheep, ferrets, swine, horses, rabbits, goats. As used herein, the terms “subject” and “patient” can also be used interchangeably. In one embodiment, a patient is a mammal such as a non-primate (e.g., cows, pigs, horses, cats, dogs, rats, etc.) and a primate (e.g., monkey and human).

As used herein, the terms “treat” and “treatment” refer to both therapeutic treatments and prophylactic or preventative measures, wherein the object is to prevent or attenuate an undesired physiological condition, disorder or disease or obtain beneficial or desired clinical results. For purposes of this invention, beneficial or desired clinical results include but are not limited to, alleviation of symptoms; diminishment of extent of condition, disorder or disease; stabilized (i.e., not worsening) state of condition, disorder or disease; delay or slowing of condition, disorder or disease progression; amelioration of the condition, disorder or disease state; remission (whether partial or total), whether detectable or undetectable; or enhancement or improvement of condition, disorder or disease. Treatment includes eliciting a cellular response that is clinically significant, without excessive levels of side effects. Treatment also includes prolonging survival as compared to expected survival if not receiving treatment.

As used herein, the phrase “electrolytic drug” refers to the pharmaceutically acceptable ionic form of a drug that is capable of being ionized by dissociation or protonation.

As used herein, the term “drug” means an active ingredient, e.g., a therapeutically active ingredient; and the terms “drug”, “active ingredient”, and “active pharmaceutical ingredient” (or “API”) are used interchangeably.

As used herein, the terms “extended release phase” or “extended release portion” refer to the phase or portion of a drug composition which undergoes sustained release over time upon administration of the composition to a patient; and in a liquid form controlled release composition, such terms refer to the dispersed solid phase of the composition, i.e., the dispersed phase.

As used herein, the terms “immediate release phase” or “immediate release portion” refer to the phase or portion of a drug composition which undergoes immediate release upon administration of the composition to a patient; and in a liquid form controlled release composition, such terms refer to the liquid phase of the composition, i.e., the dispersion medium.

As used herein, the term “diffusible counterion” refers to a pharmaceutically acceptable ion that is capable of displacing or replacing the electrolytic drug from the ion-exchange matrix. If the diffusible counterion has a positive charge, it is referred to herein as a diffusible counter-cation. Non-limiting examples of diffusible counter-cations such as, e.g., sodium, potassium, magnesium or calcium. If the diffusible counterion has a negative charge, it is referred to herein as a diffusible counter-anion. Non-limiting examples of diffusible counter-anions include, e.g., chloride, bromide, iodide, and phosphate.

As used herein, the term “water soluble” when used in connection with an electrolytic drug means having a solubility of greater than about 3 g of the electrolytic drug in 100 ml of water at any physiologically relevant pH. In particular embodiments, the term water soluble means having a solubility of greater than 1 g of the electrolytic drug in 100 ml of water at any physiologically relevant pH.

As used herein, the phrase “substantially free of diffusible counterions” when used to describe the concentration or presence of diffusible counterions in the dispersion medium means the concentration of diffusible counterions in the dispersion medium is less than about 0.1-0.5 moles per liter of liquid, less than about 0.05-0.1 moles per liter of liquid, less than about 0.01-0.5 moles per liter of liquid, or less than about 0.01 moles per liter of liquid.

As used herein, the phrase “base form of the amine” when used in connection with a drug or an ion-exchange matrix means that substantially all the amine-nitrogen atoms are unprotonated and have a neutral charge.

As used herein, the phrase “acid form” when used in connection with a drug or an ion-exchange matrix means that substantially all the acid groups are in their undissociated, uncharged acid form.

As used herein, the term “polyelectrolyte” means a molecule having a charge on its surface. In certain embodiments, a polyelectrolyte has a molecular weight large enough that it will not substantially diffuse through the optional diffusion-controlling membrane such as those useful for the present invention. The term “polyelectrolyte” encompasses molecules, oligomers, co-oligomers, polymers, co-polymers, each of which may be organic, inorganic or a combination thereof.

As used herein, the term “hydrophilic colloid” encompasses large organic molecules capable of being solvated or associated with the molecules of the dispersion medium. Hydrophilic colloid also encompasses a colloidal dispersion in which the dispersed particles are more or less liquid and exert a certain attraction on and adsorb a certain quantity of the fluid in which they are suspended.

As used herein, the term “diffuse double layer” means the molecularly dynamic region in the immediate vicinity of the charged surface of a dispersed or suspended solid or liquid phase, such dispersed material usually having a particle size that falls within the colloidal range, that contains both ions with opposite and similar charge to that of the colloidal surface, but possessing overall, in the extent of the diffuse double layer, an excess concentration of oppositely charged ions (i.e., counterions) sufficient to neutralize the surface charge of the dispersed phase.

As used herein, the phrase “uncomplicated gel” refers, in part, to the absence of spectator ions in an aqueous gel.

As used herein, the phrase “highly hydrated” describes a component having sufficient hydrogen bonds to restrict the thermodynamic activity of water.

### 5.2 Abbreviations

| Abbreviation | Definition |
|---|---|
| ACCP | American College of Chest Physicians |
| ADME | Absorbtion, Distribution, Metabolism, Excretion |
| API | Active Pharmaceutical Ingredient |
| AUC | Area under the concentration versus time curve from time 0 |
| | to infinity |
| AUCₜ | Area under the concentration versus time curve from time 0 |
| | to the last measured concentration (Cₜ) |
| BA | Bioavailability |
| BE | Bioequivalent |
| Cₘₐₓ | Maximum plasma concentration; the highest concentration |
| | observed during a dosage interval |
| Cₘᵢₙ | Minimum plasma concentration; the lowest concentration |
| | observed during a dosage interval |
| CPM | Chlorpheniramine maleate |
| CNS | Central Nervous System |
| ER | Extended Release |
| FDA | U.S. Food and Drug Administration |
| GRASE | Generally Recognized as Safe and Effective |
| HC | Hydrocodone |
| IND | Investigational New Drug |
| IR | Immediate Release |
| OTC | Over-the-counter |
| PSE | Pseudoephedrine |
| RLD | Reference Listed Drug |
| PK | Pharmacokinetic |
| SAE | Serious adverse event |
| t_{1/2} | Terminal half-life; |
| tₘₐₓ | The time that Cₘₐₓ was observed |

### 5.3 Sustained Release Technology of the Invention

### 5.3.1 The Dosage Form of the Invention

As provided above, the invention described and claimed herein comprises a novel class of sustained release drug formulations comprising a drug confined in a dispersed phase of a suspension by an ion-exchange matrix having a surface charge opposite that of the drug.

The invention specifically encompasses liquid sustained release formulations comprising drug-loaded beads produced by combining an ion-exchange matrix that is a hydrophilic colloid and a drug having a charge opposite that of the matrix in the presence of water.

The liquid sustained release formulations of the invention are capable of controlled release spanning about 8 hours, about 10 hours, about 12 hours, about 16 hours, about 18 hours, about 24 hours, or up to about 48 hours. In certain embodiments, from about 15%, 20%, 25%, 30%, 35%, 40%, or 45% and up to about 50%, 55%, 60%, 65%, 70%, 75%, 80%, 85%, 90%, 95% or 100% of the total molar amount of a drug in a liquid sustained release formulation of the invention is released over the time period of 12 hours, 16 hours, or 24 hours upon administration of such formulation to a patient. In some embodiments, from about 40% to about 100%, or 40% to 60%, or 45% to 55%, of the total molar amount of a drug in a liquid sustained release formulation of the invention is released over the time period of 12 hours upon administration of such formulation to a patient. In certain embodiments, from about 40% to about 100%, or 40% to 60%, or 45% to 55%, of the total molar amount of a drug in an extended release phase of a liquid sustained release formulation of the invention is released over the time period of 12 hours upon administration of such formulation to a patient. In other embodiments, from about 40% to about 100%, or 70% to 100%, or 90% to 100%, of the total molar amount of a drug in a liquid sustained release formulation of the invention is released over the time period of 24 hours upon administration of such formulation to a patient. In certain embodiments, from about 40% to about 100%, or 70% to 100%, or 90% to 100%, of the total molar amount of a drug in an extended release phase of a liquid sustained release formulation of the invention is released over the time period of 24 hours upon administration of such formulation to a patient.

In certain embodiments, the liquid sustained release drug delivery systems comprise beads which contain a drug and are coated with a material that controls the release of the drug. In a specific embodiment, the coating is a barrier through which the drug must diffuse before it becomes bioavailable.

In certain embodiments, the drug is in its base form. The hydrophilic colloids contemplated by the instant invention have acidic and basic functional groups which can be ionized under certain conditions which leads to extensive hydration characterized by strong ion-dipole interactions with water. As such the ion-exchange matrix interaction with the drug results in spontaneous and reversible formation of uncomplicated aqueous gels. Such an interaction enhances thermodynamic stability by virtue of attractive electrostatic forces. Other advantages conferred by such interactions include but are not limited to high viscosity and electrophoretic mobility. The aqueous gel resulting from the interaction is suitable for granulation and spheronization/extrusion. In one embodiment, the resulting gel is used to form beads. The resulting drug-loaded beads are very easily manufactured with few critical variables that require exact control. The beads are hydrophilic with content uniformity. The beads are uniquely suitable for incorporation into sustained release liquid suspension dosage forms. A suitable polymer coating on the bead can confine the drug inside the bead because of electrical neutralizing constraints associated with the inability of the drug complex to permeate the membrane. The drug remains associated with the ion-exchange matrix until encountering other components that encourage drug diffusion. As such, in certain embodiments, the dispersed phase is membrane-coated.

In other embodiments, the drug is in its salt form, e.g., a pharmaceutically acceptable salt form. Suitable pharmaceutically acceptable salts of drugs include, but are not limited to, sodium, potassium, lithium; calcium, magnesium; aluminum and zinc or other similar metals; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine; also sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate, hydrochloride, pamoate (i.e., 1,1′-methylene-bis-(2-hydroxy-3-naphthoate), embonate, estolate, and tosylate. As such, use of salt forms of drugs provide multiple advantages. A drug substance often has certain suboptimal physicochemical or biopharmaceutical properties that can be overcome by pairing an ionized basic or acidic drug molecule with a counterion to create a salt version of the drug. In addition, pharmaceutically acceptable drugs, suitable for use in humans, are generally more easily available from manufacturers in salt forms rather than base forms. The choice of salt is governed largely by the acidity or basicity of the ionizable group, the safety of the counterion, the drug indications and the intended dosage form. One skilled in the art would know how to select a pharmaceutically acceptable salt form of a drug (see, e.g., Kumar, L., et al., “Salt Selection in Drug Development,” in Pharmaceutical Technology 3(32) (2008)).

In certain embodiments, the dispersion medium is substantially free of diffusible counterions, and optionally includes a polyelectrolyte with the same charge as the drug in a dispersion medium. Drug “release” is triggered when the suspension is placed in an environment, for example gastric or intestinal fluid, with a high concentration of water and small ions (i.e., capable of diffusing through and/or displacing the drug from the ion exchange matrix) that possess the same charge as the drug. These small ions or counter ions swamp the ion-exchange matrix drug complex and cause release of the electrolytic drug into the dispersed phase of gastrointestinal fluid. Thus, the concentration of electrolytic drug in the dispersion medium is effected, in part, by the rate and extent to which the ion-exchange matrix influences diffusion into the dispersion medium. The rate and extent to which the ion-exchange matrix influences diffusion can affect the sustained release profile of the drug.

In alternate embodiments, the dispersion medium comprises a component(s) that is highly hydrated and capable of associating with water. Such components attract water from the dispersion medium such that water activity outside the bead and in the dispersion medium is less than inside the bead. In various embodiments, the dispersion medium has low enough water activity to preclude water diffusion into the drug-loaded bead and the development of internal osmotic pressure until the formulation is administered and the dispersion medium is diluted. In certain embodiments, the component is a non-electrolytic excipient such as, but not limited to, sucrose, dextrose, maltose, mannitol, sorbitol, glycerin, or low molecular weight polyethylene glycol. In certain such embodiments, the drug delivery systems contemplated by the invention are activated by water (e.g., water in gastric fluid). In such embodiments, when the activity of water outside the bead in the dispersion medium is greater than the activity of water inside the bead, water will diffuse through the coating and dissolve soluble components of the bead and create a reservoir of diffusible free drug. Such free drug can permeate the coating which can control release of the drug to the patient.

In one embodiment, the dispersion medium does not comprise hydroxypropylmethyl cellulose (“HPMC”).

As such, it is an object of the invention to provide such a novel class of drug delivery system comprising a drug confined in a dispersed phase of a suspension, which comprises an ion-exchange matrix with a charge opposite that of the drug.

In certain embodiments, the composition of the present invention, particularly the dispersion medium, also comprises a polyelectrolyte having a surface charge like that of the electrolytic drug. Without being limited by theory, Applicants believe that release of the electrolytic drug from the dispersed phase is controlled or delayed by repulsive interactions between the polyelectrolyte and the electrolytic drug, since both have the same charge. This “trapping” of the electrolytic drug allows for the administration of the ionized form of drugs with high water solubility, and provides a low concentration of the electrolytic drug in the dispersion medium.

In one embodiment, the present invention relates to a liquid form controlled release drug composition, comprising:

(a) a dispersed phase comprising a water-soluble electrolytic drug associated with a pharmaceutically acceptable ion-exchange matrix, wherein the surface charge of the ion-exchange matrix is opposite that of the electrolytic drug; and

(b) a dispersion medium that is substantially free of diffusible counterions.

In one embodiment, the present invention relates to a liquid form controlled release drug composition, comprising:

(a) a dispersed phase comprising a water-soluble electrolytic drug associated with a pharmaceutically acceptable ion-exchange matrix, wherein the surface charge of the ion-exchange matrix is opposite that of the electrolytic drug; and

(b) a dispersion medium comprising a polyelectrolyte having the same charge as the electrolytic drug.

In one embodiment, the present invention relates to a liquid form controlled release drug composition, comprising:

(a) a dispersed phase comprising an ion-exchange matrix drug complex comprising a pharmaceutically acceptable ion-exchange matrix and a water-soluble electrolytic drug associated with the ion-exchange matrix, wherein the surface charge of the ion-exchange matrix is opposite that of the electrolytic drug; and

(b) a dispersion medium that is substantially free of diffusible counterions.

In one embodiment, the present invention relates to a liquid form controlled release drug composition, comprising:

(a) a dispersed phase comprising an ion-exchange matrix drug complex comprising a pharmaceutically acceptable ion-exchange matrix and a water-soluble electrolytic drug associated with the ion-exchange matrix, wherein the surface charge of the ion-exchange matrix is opposite that of the electrolytic drug; and

(b) a dispersion medium comprising a polyelectrolyte having the same charge as the electrolytic drug.

In another embodiment, the present invention relates to a liquid form controlled release drug composition, comprising:

(a) a dispersed phase comprising two or more active ingredients in an extended release form, wherein the dispersed phase comprises particulates, pellets or beads; wherein two or more active ingredients associate in a single particulate, pellet or bead; and

(b) a dispersion medium.

In a specific embodiment, the present invention relates to a liquid form controlled release drug composition, comprising:

(a) a dispersed phase comprising two or more active ingredients in an extended release form, wherein the dispersed phase comprises particulates, pellets or beads; wherein two or more active ingredients associate in a single particulate, pellet or bead;

(b) a diffusion-controlling membrane coating; and

(c) a dispersion medium.

In some of these embodiments, the dispersed phase further comprises an ion-exchange matrix drug complex comprising a pharmaceutically acceptable ion-exchange matrix and a water-soluble electrolytic drug associated with the ion-exchange matrix, wherein the surface charge of the ion-exchange matrix is opposite that of the electrolytic drug. In some embodiments, the ion-exchange matrix is sodium alginate. In one such embodiment, at least one of the active ingredients is in a salt form.

In yet other embodiments, the dispersion medium comprises one or more active ingredients in an immediate release form. In one embodiment, the dispersion medium does not contain an ion-exchange matrix. In yet another embodiment, the dispersion medium does not comprise an ion-exchange matrix with the surface charge opposite that of at least one drug of one or more drugs in the dispersion medium. Specifically, in such embodiments, at least one, or all, of the active ingredients present in the dispersion medium is/are not bound to an ion-exchange matrix. Further, in some embodiments, at least one of the active ingredients in the dispersion medium is in a salt form. In other embodiments, at least one of the active ingredients in the dispersion medium is not in a base form, or at least two active ingredients, at least three active ingredients, or all active ingredients in the dispersion medium are not in a base form.

In yet another embodiment, the dispersion medium comprises one or more active ingredients in an immediate release form, wherein at least one of the active ingredients in the dispersion medium is not in a salt form, or wherein two, three or all active ingredients in the dispersion medium are not in a salt form. In one such embodiment, at least one, two, three or all active ingredients in the dispersion medium are in a base form.

In one of the preferred embodiments, the compositions of the invention have a shelf life of 6 months or more. In certain embodiments, the compositions of the invention maintain stability prior to administration to a patient for 6 months or more.

Yet further, in some compositions of the invention, the diffusion-controlling membrane is selected from the group consisting of ethylcellulose, methylmethacrylate, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, and combinations thereof. In one embodiment, the diffusion-controlling membrane is ethylcellulose, methylmethacrylate, or combinations thereof. In another embodiment, the diffusion-controlling membrane coating is from about 20% to about 30% by weight based on the total weight of the coating and the ion-exchange matrix drug complex.

Further, in another embodiment, the dispersion medium comprises a highly hydrated excipient. Specifically, in one embodiment, the dispersion medium comprises 50% to 70% on a weight by weight basis of a highly hydrated excipient. In yet another embodiment, the highly hydrated excipient is sucrose.

In certain embodiments, the dispersion medium comprises a solution of 50% to 70% on a weight by weight basis of a highly hydrated excipient. In such embodiment, the highly hydrated excipient is completely dissolved. In one such embodiment, the highly hydrated excipient is sucrose. In one such embodiment, the dispersion medium comprises a solution of 50% to 70% on a weight by weight basis of sucrose, wherein the sucrose is dissolved. These embodiments are based on the inventor's discovery that dissolved highly hydrated excipient, e.g., dissolved sucrose, in the dispersion medium allows to achieve desired drug release and stability characteristics of the liquid form controlled release drug compositions of the invention. In certain embodiments, the dispersion medium does not comprise any undissolved highly hydrated excipient, e.g., undissolved sugar or sucrose.

The compositions of the invention, in one embodiment, further comprise an excipient selected from the group consisting of sweetening agents, flavoring agents, coloring agents, and any combination thereof. In another embodiment, the composition, further comprises a dispersion additive selected from the group consisting of stabilizing agents, dispersion agents, and any combination thereof.

Suitable examples of the families of drugs for use in the present invention include the following.

Alpha-adrenergic agonists that can be used include, but are not limited to adrafinil, adrenolone, amidephrine, apraclonidine, budralazine, clonidine, cyclopentamine, detomidine, dimetofrine, dipivefrin, ephedrine, epinephrine, fenoxazoline, guanabenz, guanfacine, hydroxyamphetamine, ibopamine, indanazoline, isometheptene, mephentermine, metaraminol, methoxamine, methylhexaneamine, metizoline, midodrine, modafinil, moxonidine, naphazoline; norepinephrine, norfenefrine, octodrine; octopamine, oxymetazoline, phenylephrine hydrochloride, phenylpropanolamine hydrochloride; phenylpropyl-methylamine, pholedrine, propylhexedrine, pseudoephedrine, rilmenidine, synephrine, talipexole, tetrahydrozoline, tiamenidine, tramazoline, tuaminoheptane, tymazoline, tyramine, and xylometazoline.

Beta-adrenergic agonists that can be used include albuterol, bambuterol, bitolterol, carbuterol, clenbuterol, clorprenaline, denopamine, dioxethedrine, dopexamine, ephedrine, epinephrine, etafedrine, ethylnorepinephrine; fenoterol, formoterol, hexoprenaline; ibopamine, isoetharine, isoproterenol, mabuterol, metaproterenol, ethoxyphenamine, oxyfedrine, pirbuterol, prenalterol, procaterol, protokylol, reproterol, rimiterol, ritodrine, salmeterol, soterenol terbutaline, tretoquinol, tulobuterol, and xamoterol.

Alpha-adrenergic blockers that can be used include amosulalol, rotinolol, dapiprazole, doxazosin, ergoloid mesylates, fenspiride, indoramin, labetalol, naftopidil, nicergoline, prazosin, tamsulosin, terazosin, tolazoline, trimazosin, and yohimbine.

Beta-adrenergic blockers that can be used include acebutolol, alprenolol, amosulalol, arotinolol, atenolol, befunolol, betaxolol, bevantolol, bisoprolol, bopindolol, bucumolol, bufetolol, bufuralol, bunitrolol, bupranolol, butidrine hydrochloride, butofilol, carazolol, carteolol, carvedilol, celiprolol, cetamolol, cloranolol, dilevalol, epanolol, esmolol, indenolol, labetalol, levobunolol, mepindolol, metripranolol, metoprolol, moprolol, nadolol, nadoxolol, nebivalol, nifenalol, nipradilol, oxprenolol, penbutolol, pindolol, practolol, pronethalol, propranolol, sotalol, sulfinalol, talinolol, tertatolol, tilisolol, timolol, toliprolol, and xibenolol.

Narcotic analgesics that can be used include alfentanil, benzylmorphine, codeine, codeine methyl bromide; codeine phosphate, codeine sulfate, desomorphine, dihydrocodeine, dihydrocodeinone enol acetate, dihydromorphine, ethylmorphine, hydrocodone, hydromorphone, methadone hydrochloride, morphine, morphine hydrochloride, morphine sulfate, nicomorphine, normethadone, normorphine, opium, oxycodone, oxymorphone, phenoperidine, and propiram.

Non-narcotic analgesics that can be used include aceclofenac, acetaminophen, acetanilide, acetylsalicylsalicylic acid; aspirin, carbamazepine, dihydroxyaluminum acetylsalicylate, fenoprofen, fluproquazone, ibufenac, indomethacin, ketorolac, magnesium acetylsalicylate, morpholine salicylate, naproxen, phenacetin, phenyl salicylate, salacetamide, salicin, salicylamide, sodium salicylate, and tolfenamic acid.

Anorexics that may be used include a minorex, amphecloral, amphetamine, benzphetamine, chlorphentermine, clobenzorex, cloforex, clortermine, cyclexedrine, dextroamphetamine sulfate, diethylpropion, diphemethoxidine, n-ethylamphetamine, fenbutrazate, fenfluramine, fenproporex, furfurylmethyl amphetamine, levophacetoperane, mazindol, mefenorex, metamfepramone, methamphetamine, norpseudoephedrine, pentorex, phendimetrazine, phenmetrazine, phentermine, phenylpropanolamine hydrochloride, picilorex, and sibutramine.

Antiallergics that may be used include amlexanox, astemizole, azelastine, cromolyn, fenpiprane, ibudilast lodoxamide, nedocromil, oxatomide pemirolast, pentigetide, picumast, repirinast, suplatast tosylate, tazanolast, tranilast, and traxanox.

Antianginals that can be used include acebutolol, alprenolol, amiodarone, amlodipine, arotinolol, atenolol, bamidipine, bepridil, bevantolol, bucumoloL bufetolol, bufuralol, bunitrolol, bupranolol, carazolol, carteolol, celiproloL cinepazet maleate, diltiazem, elgodipine, epanolol, felodipine, gallopamil, imolamine, indenolol, isosorbide dinitrate, sradipine, limaprost, mepindolol, metoprolol, molsidomine, nadolol, nicardipine, nicorandil, nifedipine, nifenalol, nilvadipine, nipradilol, nisoldipine, nitroglycerin, oxprenolol, oxyfedrine, ozagrel, penbutolol, pentaerythritol tetranitrate, pindolol, pronethalol, propranolol, ranolzazine, somotiadil, sotalol, terodiline, timolol, toliprolol, trolnitrate phosphate, verapimil, and zatebradine.

Antiasthmatics that can be used include amlexanox, azelastine, cromolyn, ibudilast, ketotifen, montelukastnedocromil, oxatomide, pranlukast, seratrodast, suplatast tosylate, tiaramide, traxanox, zafirlukast, and zileuton.

Antibacterials or antibiotics can be used. The general classes of aminoglycosides, carbacephems, carbapenems, cephalosporins, cephamycins, penicillins, polypeptides, tetracyclines, etc. can be used. Specific antibacterials or antibiotics that can be used include amikacin, dihydrostreptomycin, kanamycin, neomycin, neomycin undecylenate; spectinomycin, streptomycin, loracarbef, biapenem, cefaclor, cefazolin, cefepime, cephalosporin C, cefbuperazone, amdinocillin, amoxicillin, ampicillin, cloxacillin, metampicillin, penicillin G benzathine, penicillin G procaine, penicillin V, piperacillin, amphomycin, vancomycin, viomycin, apicycline, chlortetracycline, methacycline, and tetracycline.

Synthetic antibacterials such as quinolones and analogs, sulfonamides, etc. can be used. Specific synthetic antibacterials that can be used include cinoxacin, lomefloxacin, nalidixic acid, oxolinic acid, acetyl sulfa-methoxypyrazine, mafenide, succinylsulfathiazole, sulfacetamide, sulfadiazine, and sulfaloxic acid.

Anticholinergics that can be used include adiphenine hydrochloride, aminopentamide, atropine, chlorphenoxamine, cyclodrine, mecloxamine pentapiperide, phencarbamide, pridinol, and scopolamine.

Antidepressants that can be used include bicyclics, hydrazides, hydrazines; pyrrolidones, tetracyclics, tricydies, etc. Specific antidepressants that can be used include binedaline, nefopam, trazodone, iproniazid, roliprani maprotiline, adinazolam, amitriptyline, clomipramine, imipramine, nortriptyline, primipramine, adrafinil, milnacipran, nefazodone, and zimeldine.

Synthetic antifungals that can be used include allylamines, imidazoles, thiocarbamates, triazoles, etc. Specific synthetic antifungals that can be used include butenafine, bifonazole, butoconazole, chlordantoin, clotrimazole, tolcielate, fluconazole, acrisorcin, exalamide, triacetin, and zinc propionate.

Nonsteroidal anti-inflammatories that can be used include aminoarylcarboxylic acid derivatives, arylacetic acid derivatives, arylbutyric acid derivatives, arylcarboxylic acids, arylpropionic acid derivatives, pyrazoles, pyrazolones, salicylic acid derivatives, thiazinecarboxamides, etc. Specific nonsteroidal anti-inflammatories that can be used include flufenamic acid, terofenamate, acemetacin, clopirac, indomethacin, metiazinic acid, fenbufen, clidanac, alminoprofen; bucloxic acid, ketoprofen, naproxen, tiaprofenic acid, difenamizole, apazone, mofebutazone, phenylbutazone, acetaminosalol, lysine acetylsalicylate, parsalmide, ampiroxicam, bendazac, nabumetone, superoxide dismutase, and zileuton.

Antispasmodics that can be used include alibendol, ambucetamide, aminopromazine, apoatropine, bevonium methyl sulfate, bietamiverine, butaverine, butropium bromide, caroverine, cimetropium bromide, cinnamedrine, clebopride, cyclonium iodide, difemerine, diisopromine, dioxaphetyl butyrate, diponium bromide, drofenine, emepronium bromide, fenalamide, fenoverine, flavoxate, flopropione, gluconicacid, hydramitrazine, hymecromone, octamylamine, pentapiperide; phloroglucinol, pinaverium bromide, piperilate; prifinium bromide, proxazole, racefimine, rociverine; spasmolytol, sultroponium, tigloidine, tiropramide, tricromyl, trimebutine, and xenytropium bromide.

Antiulceratives that can be used include acetoxolone, aldioxa, arbaprostil, benexate hydrochloride, carbenoxolone, cetraxate, cimetidine, colloidal bismuth subcitrate, ebrotidine, ecabet, enprostil, esaprazole, famotidine, gefarnate, guaiazulene, irsogladine, lansoprazole, misoprostol, nizatidine, omeprazole, ornoprostil, pantoprazole, pifamine, pirenzepine, plaunotol, polaprezinc, rabeprazole, ranitidine, rebamipide, rioprostil, rosaprostol, rotraxate, roxatidine acetate, sofalcone; spizofurone, sucralfate, telenzepine, teprenone, trimoprostil, trithiozine, troxipide, and zolimidine.

Antivirals such as purines, pyrimidines, etc. can be used. Specific antivirals that can be used include acyclovir, cidofivir, cytarabine, dideoxyadenosine, didanosine, edoxudine, famciclovir, floxuridine, ganciclovir, idoxuridine, inosine pranobex, lamivudine, penciclovir, sorivudine, stavudine, zidovudine, acemannan, amantadine, amidinomycin, lysozyme; nevirapine, and ribavirin.

Anxiolytics such as arylpiperazines, benzodiazepine derivatives, carbamates, etc. can be used. Specific anxiolytics that can be used include buspirone, lesopitron, alprazolam, bromazepam, diazepam, fludiazepam, loxapine, metaclazepam, prazepam, cyclarbamate, meprobamate, abecarnil, benzoctamine, glutamic acid, mephenoxalone, and pazinaclone.

Calcium channel blockers such as arylalkylamines, dihydropyridine derivatives, piperazine derivatives, etc. can be used. Specific calcium channel blockers that can be used include bepridil, diltiazem, gallopamil, terodiline, amlodipine, benidipine, lercanidipine, nicardipine, cinnarizine, and fantofarone.

Dopamine receptor agonists can be used. Specific dopamine receptors that can be used include bromocriptine, cabergoline, carmoxirole, dopexamine, fenoldopam, ibopamine, lisuride, pergolide, pramipexole, quinagolide, ropinirole, roxindole, and talipexole.

Dopamine receptor antagonists can be used. Specific dopamine receptor antagonists that can be used include amisulpride, clebopride, domperidone, metoclopramide, mosapramine, nemonapride, romoxipride, risperidone, sulpiride, sultopride, and ziprasidone.

Narcotic antagonists can be used. Specific narcotic anagonists that can be used include amiphenazole, cyclazocine, levallorphan, nalmefene, nalorphine, naloxone, and naltrexone.

Protease inhibitors can be used. Specific protease inhibitors that can be used include aprotinin, camostat, gabexate, nafamostat, and urinastatin.

Respiratory stimulants can be used. Specific respiratory stimulants that can be used include almitrine, bemegride, cropropamide, crotethamide, dimefline, dimorpholamine, doxapram, ethamivan, fominoben, lobeline, mepixanox, nikethamide; picrotoxin, pimeclone, pyridofylline, sodium succinate, andtacrine.

Retroviral protease inhibitors can be used. Specific retroviral protease inhibitors that can be used include indinavir, and ritonavir.

Reverse transcriptase inhibitors can be used. Specific reverse transcriptase inhibitors that can be used include delavirdine, didanosine, dideoxyadenosine, foscarnet sodium, lamivudine, nevirapine, stavudine, suramin sodium, zalcitabine; and zidovudine.

Sedatives such as benzodiazepine derivatives can be used. Specific sedatuves that can be used include brotizolam, cinolazepam, doxefazepam, estazolam, flunitrazepam, flurazepam, haloxazolam, loprazolam, lormetazepam, nitrazepam, quazepam, temazepam, and triazolam.

Cerebral vasodilators can be used. Specific cerebral vasodilators that can be used include bencyclane, cinnarizine, citicoline, cyclandelate, ciclonicate, ebumamonine, fasudil, fenoxedil, flunarizine, ibudilast, ifenprodil, lomerizine, nafronyl, nicametate, nicergoline, nimodipine, papaverine, pentifylline, tinofedrine, vincamine, vinpocetine, and viquidil.

Coronary vasodilator can be used. Specific coronary vasodilators that can be used include amotriphene, bendazol, benfurodil hemisuccinate, benziodarone, chloracizine, chromonar clobenfurol, clonitrate, cloricromen, dilazep, dipyridamole, droprenilamine, efloxate, erythrityl tetranitrate, etafenone, fendiline, floredil, ganglefene; heart muscle extract, hexobendine, itramin tosylate, khellin, lidoflazine, mannitol hexanitrate, medibazine; pentaerythritol tetranitrate, pentrinitrol, perhexyline, pimethylline, prenylamine, propatyl nitrate, pyridofylline, trapidil, tricromyl, trimetazidine; trolnitrate phosphate, and visnadine.

Peripheral vasodilator can be used. Specific peripheral vasodilators that can be used include bamethan, bencyclane, betahistine, bradykinin, brovincamine, bufeniode, buflomedil, butalamine, cetiedil, ciclonicate, cinepazide; cyclandelate, eledoisin, fenoxedil, flunarizine, hepronicate, ifenprodil, iloprost, inositol niacinate, isoxsuprine, kallidin, kallikrein, moxisylvyte, nafronyl, nicametate, nicergoline, nicofuranose, nicotinyl alcohol, nylidrin, pentifylline, pentoxifylline, piribedil, suloctidil, tolazoline, and xanthinol niacinate.

Antiamebics that can be used include arsthinol, bialamicol, carbarsone, cephaeline, chlorbetamide, chloroquine, chlorphenoxamide, chlortetracycline, dehydroemetine, dibromopropamidine, diloxanide, diphetarsone, emetine, fumagillin, glaucarubin, iodoquinol, paromomycin, phanquinone, polybenzarsol, propamidine, quinfamide, secnidazole, sulfarside, teclozan, tetracycline, thiocarbamizine, thiocarbarsone, and timidazole.

In one embodiment of the compositions of the present invention, the one or more of the active ingredients are selected from a cardiovascular drug, respiratory drug, sympathomimetic drug, cholinomemetic drug, adrenergic drug, antimuscarinic drug, antispasmodic drug, skeletal muscle relaxant, diuretic drug, anti-migraine drug, anesthetic, sedative, hypnotic, antiepileptic, psychopharmacologic agent, analgesic, including opioid and non-opioid analgesic, antipyretic, CNS stimulant, antineoplastic, antiemetic, immunosuppressive drug, antimicrobial drug, antihistamine, anti-inflammatory, antibiotic, decongestant, cough suppressant, expectorant or a combination thereof. In one embodiment one or more of the active ingredients are selected from the group of an antihistamine, an antitussive, a decongestant, an antiemetic, and a sedative. In a specific embodiment, the active ingredients comprise an antihistamine, an antitussive, and optionally a decongestant. In a certain specific embodiment, the two or more active ingredients comprise chlorpheniramine, hydrocodone and optionally pseudoephedrine. In another embodiment, the two or more active ingredients consist of an antihistamine, an antitussive, and optionally a decongestant. In a certain specific embodiment, the two or more active ingredients consist of chlorpheniramine, hydrocodone and optionally pseudoephedrine. In one embodiment, the two or more active ingredients consist of chlorpheniramine, hydrocodone and pseudoephedrine.

Further, in another embodiment of a liquid form controlled release drug composition, administration of a single dose of the drug composition to a patient provides serum levels of the active ingredients over a time period of at least 8 hours that are bioequivalent to serum levels achieved upon administration of an appropriate number of doses, over the same time period, of FDA-approved immediate release reference listed drug (IR RLD) compositions comprised of the active ingredients, and the appropriate number of doses corresponds to a number of doses recommended in one or more FDA-approved labels for administration of the IR RLD compositions over the same time period.

In yet another embodiment, the present invention relates to a liquid form controlled release drug composition, comprising:

(a) a dispersed phase comprising one or more active ingredients in an extended release form, and

(b) a dispersion medium comprising one or more active ingredients in an immediate release form.

In some embodiments, the present invention relates to a liquid form controlled release drug composition, comprising:

(a) a dispersed phase comprising two or more active ingredients in an extended release form, wherein the dispersed phase comprises particulates, pellets or beads; wherein two or more active ingredients associate in a single particulate, pellet or bead; and

(b) a dispersion medium comprising one or more active ingredients in an immediate release form.

In another embodiment, the dispersed phase further comprises a pharmaceutically acceptable ion-exchange matrix and a water-soluble electrolytic drug(s) associated with the ion-exchange matrix, wherein the surface charge of the ion-exchange matrix is opposite that of the electrolytic drug. In another embodiment, the dispersed phase is membrane-coated. In particular embodiments, the membrane is polymeric. The membrane can be porous or non-porous. In one preferred embodiment, the membrane is porous. In such particular embodiment, the membrane controls diffusion of the drug. In such embodiment, the porous membrane coating enables slow release of a drug in the liquid form controlled release drug composition of the invention. In another preferred embodiment, the membrane coating is not non-porous. In yet another preferred embodiment, the dispersed phase of the liquid form controlled release compositions of the invention is not coated with a coating that does not allow diffusion of the drug from the dispersed phase, e.g., a non-porous membrane coating. In yet other embodiments, the dispersed phase comprises drug-loaded beads that include a low molecular weight, non-electrolytic soluble excipient(s) capable of dissolving and diffusing out of the beads when water is absorbed into the bead and reducing osmotic pressure inside the beads. In one of the embodiments, the low molecular weight excipient is lactose. In another embodiment, the dispersion medium further comprises a highly hydrated excipient that attracts water in the dispersion medium. The invention contemplates a high concentration of a highly hydrated excipient, e.g., 50% to 70%, or 55% to 70%, or 45% to 70%, or 55% to 65%, or 60% to 70% on a weight by weight basis of a highly hydrated excipient in the dispersion medium. In one of the embodiments, the highly hydrated excipient in the dispersion medium is sucrose, for example 65% sucrose on a weight by weight basis. In a certain embodiment, one or more drugs or active ingredients in a dispersed phase and/or dispersion medium are not in a base form. In another embodiment, one or more drugs or active ingredients in a dispersed phase and/or dispersion medium are in a salt form. In yet another embodiment, the dispersed phase comprises a mixture of drug and ion-exchange matrix powders, wherein the drug(s) and the ion-exchange matrix are in a salt form, e.g., a pharmaceutically acceptable salt form.

In yet another embodiment, one or more drugs or active ingredients in a dispersed phase and/or dispersion medium is not in a salt form. In one such embodiment, one or more drugs or active ingredients in a dispersed phase and/or dispersion medium are in a base form.

The ion-exchange matrix can be a high molecular weight organic compound such as an oligomer, co-oligomer, polymer or co-polymer; a porous inorganic network solid such as, e.g., a zeolite; and/or combinations thereof, which have charged surfaces and are capable of retaining an oppositely-charged ion. As used herein, the phrase “cation-exchange matrix” refers to an ion exchange matrix which is capable of retaining a cationic form of a drug. As used herein, the phrase “anion-exchange matrix” refers to an ion exchange matrix which is capable of retaining an anionic form of a drug.

The design of the system and the selection of appropriate components are predicated on the charge of the therapeutically active ingredient (drug). The invention is suitable for the administration of drugs which are uncharged bases or acids; or cationic or anionic drugs, which are strong electrolytes as well as weakly acidic drugs above their pKa (anions) and weakly basic drugs below their pKa (cations). When the drug is an ion, the ion-exchange matrix must have a charge opposite that of the drug ion. When the drug is an uncharged base or acid, the ion-exchange matrix is in the form of an acid or base, respectively. If the electrolytic drug is a cation, then an ion-exchange matrix with a negative surface functionality must be utilized as ion-exchange matrix. If the drug is an anion, then an ion-exchange matrix with a positive surface functionality must be employed. Examples of suitable pharmaceutical ingredients that may be used to form the core in this case include but are not limited to chitosan, polylysine, and gelatin.

Cation- and anion-exchange matrices are well-known in the art. Non-limiting examples of useful cation-exchange matrices include cation-exchange resins such as, e.g., resins having polymer backbones comprising styrene-divinyl benzene copolymers and having pendant sulfonate groups, available from Rohm and Haas, Philadelphia, Pa., and sold under the tradename AMBERLITE™ IRP69; methacrylic acid and divinyl benzene co-polymers which have a carboxylate functionality, available from Rohm and Haas, and sold under the tradenames AMBERLITE™ IRP64 and IRP88; hydrophilic colloids such as, e.g., alginate, carboxymethylcellulose, croscarmellose, microcrystalline cellulose, xanthan gum, carboxy vinyl polymers such as carbomer 94, gelatin; or any combination thereof. In one embodiment, the cation-exchange matrix is alginate, carboxymethylcellulose, microcrystalline cellulose, xanthan gum, carboxy vinyl polymer, gelatin or any combination thereof.

Non-limiting examples of useful anion-exchange matrices include anion-exchange resins such as, e.g., resins having polymer backbones comprising styrene-divinyl benzene copolymers and having pendant ammonium or tetraalkyl ammonium functional groups, available from Rohm and Haas, Philadelphia, Pa., and sold under the tradename DUOLITE™ AP143; and hydrophilic colloids such as, but not limited to, chitosan, polylysine, or gelatin; and any combination thereof. In one embodiment, the anion-exchange matrix is chitosan, polylysine, gelatin or any combination thereof.

In certain embodiments, the ion-exchange matrix is water-insoluble. In an alternate embodiment, the ion-exchange matrix is water soluble. In such embodiments, the ion-exchange matrix is capable of being solvated with the dispersion medium, and is preferably a hydrophilic colloid. The invention contemplates hydrophillic colloids including but not limited to natural materials such as starch, agar, cellulose, alginic acid, guar gum, xanthan gum, gelatin, acacia, and albumin have been used for applications that range from something as simple as a wet binder to something as novel as a component of microspheres. Non-limiting synthetic examples include methylcellulose, carboxymethylcellulose, hydroxypropylmethyl cellulose, methylacrylic acid, polylactic acid, polyglycolic acid, and polyanhydrides are also widely deployed in non-limiting applications ranging from the traditional, such as wet granulation, to the more contemporary, such as functional coatings and biodegradable implants.

In certain embodiments, the cation-exchange matrix is a hydrophillic colloid. In such embodiments, the cation-exchange matrix is alginate, carboxymethylcellulose, microcrystalline cellulose, xanthan gum, carboxyvinyl polymers such as carbomer 94, or any combination thereof. In certain embodiments, the hydrophilic colloid is cross-linked to reduce swelling. In one embodiment, the ion-exchange material is calcium alginate. In another embodiment, the ion-exchange matrix is sodium alginate.

In other embodiments, the anion-exchange matrix is a hydrophillic colloid. In such embodiments, the anion-exchange matrix is chitosan, polylysine, gelatin, or any combination thereof. In certain embodiments, the hydrophilic colloid is cross-linked to reduce swelling.

The water soluble electrolytic drug associates with the ion-exchange matrix and forms an ion-exchange matrix drug complex. Without being bound by any particular theory, Applicants believe that one advantage of the present invention stems from the electrostatic interactions between the drug and the ion-exchange matrix, which circumvents many of the traditional challenges faced when formulating liquid sustained release oral dosages.

In certain embodiments, the ion-exchange matrix drug complex is in the form of a particulate or bead. The particulate or bead is of a size which can be administered orally in a liquid dosage form. In one embodiment, the particulate or bead is of a size and/or density such that it does not settle in suspension. In certain embodiments, the particulate or bead does not have undesirable patient attributes. In particular embodiments, the diameter of the particulate or bead ranges from about 0.01 μm to about 2000 μm; in another embodiment, from about 0.1 μm to about 1000 μm; and in another embodiment, from about 1 μm to about 1000 μm. In other embodiments, the diameter of the particulate or bead is greater than 2000 μm, greater than 3000 μm, or greater than 5000 μm. In alternate embodiments, the diameter of the particulate or bead is no greater than 2000 μm, no greater than 1000 μm, no greater than 500 μm, no greater than 50 μm, or no greater than 1 μm. In one embodiment, the diameter of the particulates, pellets or beads is about 600 μm. In some embodiments, the diameter of the particulates, pellets or beads is about 200 μm, about 300 μm, about 400 μm, about 500 μm, about 600 μm, about 700 μm, about 800 μm, or about 900 μm.

The core may further comprise pharmaceutically acceptable processing aid useful for forming solid dosage forms including, but limited to, bulking agents such as starch, titanium oxide, and silica; preservatives; stabilizers such as antioxidants; lubricants such as vegetable oils; and the like.

In certain embodiments, the ion-exchange matrix drug complex further comprises a low molecular weight, soluble, non-electrolytic excipient. Such an excipient is capable of dissolving in water and diffusing out of the bead when the bead absorbs water and thereby reduces osmotic pressure inside the bead. The excipient must have a low enough molecular weight to permeate any membrane coating the bead. In various embodiments, the amount of excipient included in the bead can affect the rate of drug release. In various embodiments, the excipient is present in the bead at about 5% to about 10%, at about 10% to about 20%, at about 20% to about 30% at about 30% to about 40%, at about 40% to about 45%. In a specific embodiment, the excipient is lactose. In certain such embodiments, the more lactose incorporated in the dispersed phase during manufacturing, the faster the release of drug from the bead after administration. In various embodiments, lactose is present in the bead at about 5% to about 10%, at about 10% to about 20%, at about 20% to about 30% at about 30% to about 40%, at about 40% to about 45%. In certain embodiments, lactose is present in the bead at about 20% to about 30%. Other examples of soluble non-electrolytic excipients encompassed by the invention include but are not limited to dextrose, maltose, mannitol, sorbitol, glycerin, or low molecular weight polyethylene glycol.

In one embodiment, the ion-exchange matrix drug complex further comprises a diffusion-controlling membrane coating. The membrane coating is useful for further controlling diffusion of counterions into and drug out of the ion-exchange matrix. Thus, the diffusion-controlling membrane coating is useful for controlling the release of the electrolytic drug into the dispersion medium and/or the digestive tract after administration to a patient. The invention encompasses the use of any membrane-coating that provides diffusion control. The coating materials may be any of a large number of natural or synthetic film-formers used alone, in admixture with each other, and in admixture with other components such as plasticizers, pigments, and other substances. The components of the coating are preferably insoluble in, and permeable to, water. Incorporation of a water-soluble substance can be useful in altering the permeability of the coating. Diffusion-controlling membranes are known in the art. Non-limiting examples include ethylcelluloses such as SURELEASE® (Colorcon, Westpoint, Pa.); methylmethacrylate polymers such as EUDRAGIT® (Rohm Pharma, GmbH, Weiterstat, Germany); cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, and cellulose acetate butyrate. In one embodiment, the coating is a methylmethacrylate polymer.

In one embodiment, the diffusion-controlling membrane is selected from the group consisting of ethylcellulose, methylmethacrylate, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, and combinations thereof.

In one embodiment, the ion-exchange matrix drug complex is coated with from about 1% up to about 75% of diffusion-controlling membrane based on the total weight of the ion-exchange matrix drug complex and the diffusion-controlling membrane; in another embodiment, from 5% to about 50%; and in one of the preferred embodiments, from about 10% to about 30%, or from about 20% to about 25%. Typically, the more coating, the more delay in the release of the drug.

In one embodiment, drug-loaded alginate beads are coated with sufficient EUDRAGIT® (Rohm) RS 30 D to provide a coated bead having from about 20% to about 30% by weight of coating based on the total weight of the coating and the drug-loaded alginate beads.

In another embodiment, the diffusion-controlling membrane coating of the ion-exchange matrix drug complex further comprises a plasticizer. Plasticizers are useful to increase flexibility and reduce brittleness of the coating. Plasticizers also affect drug release rate. A plasticizer lowers the glass transition temperature of the coating and that facilitates coalescence of the applied droplets into a coherent film, and affects permeability of the coating. Plasticizers are known in the art. Non-limiting examples of plasticizers include triethyl citrate, diethyl sebacate, diethyl phthalate, tributyl citrate, and acetyl tributyl citrate. In one embodiment, the plasticizer is triethyl citrate. In one embodiment, the ion-exchange matrix drug complex contains from about 0.1% up to 30%, or from about 0.5% up to about 20%, or from about 1% to about 20%, or from about 2% to about 10%, of the plasticizer based on the total weight of the ion-exchange matrix drug complex and the diffusion-controlling membrane.

In one embodiment, the dispersion medium of the invention is a liquid with a low concentration of diffusible counterions. In such embodiment, the dispersion medium is substantially free of diffusible counterions. Useful liquids include non-aqueous pharmaceutically acceptable liquids, water, or a combination thereof. In such embodiment, the only requirement of the dispersion medium is that it has a low concentration of diffusible counterions. As used herein, the term “low” when used in connection with the diffusible counterion concentration in the dispersion medium means less than about 0.1-0.5 moles per liter of liquid, or less than about 0.05-0.1 moles per liter of liquid, or less than about 0.01-0.5 moles per liter of liquid, or less than about 0.01 moles per liter of liquid. In certain embodiments, the diffusible counterion concentration is less than 0.001-0.1 moles per liter of liquid, or no greater than 0.01 moles per liter of liquid. In certain embodiments, the dispersion medium is substantially free of diffusible counterions.

Because the concentration of diffusible counterions in the dispersion medium is low, the extent of counterion exchange with the electrolytic drug is minimized or eliminated. Thus, a substantial fraction of the electrolytic drug remains associated with the ion-exchange matrix, i.e., in the dispersed phase. In one embodiment, the amount of free drug in the dispersion medium is less than 10% based on the total molar amount of drug in the dispersion medium and dispersed phase; in another embodiment, the amount of free drug in the dispersion medium is less than 5% based on the total molar amount of drug in the dispersion medium and dispersed phase; and in another embodiment, the amount of free drug in the dispersion medium is less than 0.5% based on the total molar amount of drug in the dispersion medium and dispersed phase; and in yet another embodiment, the amount of free drug in the dispersion medium is less than 0.05% based on the total molar amount of drug in the dispersion medium and dispersed phase.

In another embodiment, the use of one or more electrolytic drugs in the dispersion medium is contemplated. Surprisingly, the composition of the present invention is stable in the presence of ionic components. In one embodiment, the composition of the present invention is stable in the presence of ionic components in the dispersion medium. In such embodiment, the composition of the present invention is stable in the presence of diffusible counterions in the dispersion medium. In another embodiment, the composition of the present invention is stable in the presence of electrolytic drugs in the dispersion medium. Surprisingly, the composition of the present invention maintains stability in the presence of a free drug or drugs in the dispersion medium, i.e., a drug or drugs that are not bound to an ion-exchange matrix. Specifically, unexpectedly the composition of the present invention maintains adequate sustained release profile of one or more drugs from the dispersed phase in the presence of free drug(s) or ionic component(s) in the dispersion medium. One of the embodiments envisages that the dispersion medium comprises at least one drug or active ingredient in an immediate release form. In another embodiment, the dispersion medium comprises two, three, four or more drugs or active ingredients in the immediate release form. In such embodiments, drug or drugs in the dispersion medium are not bound to an ion-exchange matrix. In another such embodiment, the dispersion medium does not comprise an ion-exchange matrix. In yet another such embodiment, the dispersion medium does not comprise an ion-exchange matrix with the surface charge opposite that of at least one drug of one or more drugs in the dispersion medium. In one of the embodiments, the dispersion medium Comprises one, two, three, four or more electrolytic drugs. In one such embodiment, the dispersion medium contains a pharmaceutically acceptable salt form of a drug(s). Surprisingly, such compositions of the present invention comprising a free drug or drugs in the dispersion medium have a storage shelf life at room temperature conditions of at least 1 month, of at least 3 months, of at least 6 months, or at least or about one year, two years, three, four, five or more years, during which time the stability and drug release profile characteristics of such compositions are maintained

In one of the invention's preferred embodiments, the liquid form controlled release compositions of the invention are stable in the presence of free drugs and/or diffusible counterions in the dispersion medium. In one such embodiment, the composition of the invention comprises an extended release phase and an immediate release phase, wherein the immediate phase contains a certain amount of free drug, wherein the amount of the drug released from the extended release phase into the immediate release phase before administration to a patient is less than 30%, or less than 25%, or less than 20%, or less than 10%, or less than 5%, or less than 0.5%, or less than 0.05% based on the total molar amount of drug in the dispersion medium and dispersed phase.

In some embodiments, the dispersion medium comprises one, two, three, four or more electrolytic drugs. In certain embodiments, the dispersion medium comprises two or three electrolytic drugs.

In certain embodiments of the liquid form controlled release composition of the invention comprising two or more drugs, at least one drug present in the dispersed phase and/or the dispersion medium has a therapeutic indication that is different from another drug in the dispersed phase and/or dispersion medium. A non-limiting example of such embodiments includes compositions that contain three drugs used for three different therapeutic indications, e.g., an antihistamine for the treatment of allergies and rhinorrhea, an antitussive for the treatment of cough, and a decongestant for the treatment of nasal obstruction.

In some embodiments, the dispersed phase comprises one or more drugs that are not present in the dispersion medium. In other embodiments, the dispersion medium comprises one or more drugs that are not present in the dispersed phase. For example, the first drug may be present in the dispersed phase but not the dispersion medium, and the second drug may be present in the dispersion medium but not the dispersed phase of the liquid form controlled release composition of the invention. Whereas in yet another embodiment, both the first and the second drugs may be present in both the dispersed phase and the dispersion medium.

In yet another embodiment of the liquid form controlled release composition of the invention, one or more drugs in the dispersed phase leach into the dispersion medium before its administration to a patient. In such embodiment, about 10%, 15%, 20%, 25%, 30%, 35%, 40%, 45%, 50%, 55%, 60%, 70% and up to 75% of the drug is released from the dispersed phase into the dispersion medium before its administration to a patient, based on the total molar amount of drug in the dispersion medium and dispersed phase. In one such embodiment, from about 15% to about 35%, or about 25% of the drug is released from the dispersed phase into the dispersion medium before its administration to a patient, based on the total molar amount of drug in the dispersion medium and dispersed phase.

In certain embodiments, the weight ratio of a drug in an immediate release phase to the same drug in an extended release phase of the liquid sustained release compositions of the invention (“IR/ER ratio”) is about 0:100, or about 5:100, or about 10:100, or about 15:85, or about 20:80, or about 25:75, or about 30:70, or about 35:65, or about 40:60, or about 45:50 or about 50:50. In a specific embodiment, IR/ER ratio of a drug is about 25:75. In one specific embodiment of the present invention the weight ratio of an antihistamine (e.g., chlorpheniramine) in the immediate release portion to the extended release portion of the oral composition of the invention is about 25:75, and the weight ratio of an antitussive (e.g., hydrocodone) in the immediate release portion to the extended release portion is about 25:75, and the weight ratio of a decongestant (e.g., pseudoephedrine) in the immediate release portion to the extended release portion is from about 25:75 to about 0:100.

In other embodiments, the drug composition contains from 0.1-0.5, 0.5-1 mg, 1-5 mg, 5-10 mg, 10-15 mg, 15-20 mg, 20-25 mg, 25-30 mg, 30-40 mg, 40-50 mg, 50-60 mg, 60-70 mg, 70-80 mg, 80-90 mg, 90-100 mg, 100-120 mg, 120-140 mg, 140-160 mg, 160-180 mg, 180-200 mg, 200-220 mg, 220-240 mg, 240-260 mg, 260-280 mg, 280-300 mg, 300-350 mg, 350-400 mg, 400-450 mg, 450-500 mg, up to 600 mg, up to 700 mg, up to 800 mg, up to 900 mg, up to 1000 mg of each of one or more drug/s or active ingredient's per 1 ml of the single dose of the liquid form controlled release drug composition. In yet another embodiment, the drug composition contains from 0.1-0.5, 0.5-1 mg, 1-5 mg, 5-10 mg, 10-15 mg, 15-20 mg, 20-25 mg, 25-30 mg, 30-40 mg, 40-50 mg, 50-60 mg, 60-70 mg, 70-80 mg, 80-90 mg, 90-100 mg, 100-120 mg, 120-140 mg, 140-160 mg, 160-180 mg, 180-200 mg, 200-220 mg, 220-240 mg, 240-260 mg, 260-280 mg, 280-300 mg, 300-350 mg, 350-400 mg, 400-450 mg, 450-500 mg, up to 600 mg, up to 700 mg, up to 800 mg, up to 900 mg, up to 1000 mg of each of one or more drug/s or active ingredient/s per 5 ml of the single dose of the liquid form controlled release drug composition. In some specific embodiments, the drug composition contains 1-5 mg, 5-10 mg, 10-15 mg, 15-20 mg, 20-25 mg, 25-30 mg, 30-40 mg, 40-50 mg, and up to 100-120 mg, 120-140 mg, 140-160 mg, 160-180 mg, 180-200 mg, 200-220 mg, 220-240 mg, 240-260 mg, 260-280 mg, 280-300 mg of each of one or more drug/s or active ingredient/s per 5 ml of the single dose of the liquid form controlled release drug composition.

As noted above, in specific embodiments, the liquid form controlled release drug composition comprises a dispersion medium further comprising a polyelectrolyte having the same charge as the electrolytic drug. In one embodiment, the polyelectrolyte does not displace the drug from the ion-exchange matrix. Rather, the polyelectrolyte further confines the drug in the matrix until the formulation is exposed to a high concentration of water and small ions that possess the same charge as the drug. In another embodiment, the polyelectrolyte is also a pharmaceutically acceptable molecule incapable of diffusing through a membrane. In yet another embodiment, the polyelectrolyte is capable of counteracting the effects of any diffusible counterions that may be present in the dispersion medium.

In certain preferred embodiments, the dispersion medium further comprises a high concentration of excipient(s) that are highly hydrated, capable of associating with the water in the dispersion medium. Although the drug is highly soluble in aqueous dispersion media, the presence of the highly hydrated component in the dispersion medium attracts the water in the dispersion medium necessary to begin the dissolution of drug from the drug-ion-exchange matrix complex. Only until the formulation is administered and gastric liquids (largely water) dilute the dispersion medium will drug dissolve and become available and begin to permeate the membrane and/or diffuse from the bead. In some such embodiments, the dispersion medium is substantially devoid of free drug, or has less than 0.5% drug, or less than 0.05% drug in the dispersion medium. In various embodiments, the highly hydrated component is present in the dispersion medium, on a weight to weight basis, at about 10% to about 20%, at about 20% to about 30%, at about 30% to about 40%, at about 40% to about 50%, at about 50% to about 60%. In one preferred embodiment, the dispersion medium comprises from about 50% to about 70% of the highly hydrated component. In one such embodiment, the component is present at about 60% to about 65%, up to about 70%. In specific embodiments, the dispersion medium comprises sucrose, or other sugar molecules. In such embodiments, the dispersion medium comprises, on a weight to weight basis, more than 10% sucrose, preferably more than 20% sucrose, or more than 30% sucrose, or more preferably more than 40% sucrose, or more than 50% sucrose. In one embodiment, the dispersion medium comprises about 65% sucrose (i.e., Syrup NF), but no more than about 70% sucrose. Other examples of excipients encompassed by the invention include but are not limited to dextrose, mannitol, fructose, polyethylene glycol, glycols, and glycerins. In certain embodiments, the dispersion medium comprises, on a weight to weight basis, more than 10% of dextrose, mannitol, fructose, polyethylene glycol, glycol or glycerin, preferably more than 20% dextrose, mannitol, fructose, polyethylene glycol, glycol or glycerin, or more than 30% dextrose, mannitol, fructose, polyethylene glycol, glycol or glycerin, or more preferably more than 40% dextrose, mannitol, fructose, polyethylene glycol, glycol or glycerin, or more than 50% of dextrose, mannitol, fructose, polyethylene glycol, glycol or glycerin. In one embodiment, the dispersion medium comprises about 65% dextrose, mannitol, fructose, polyethylene glycol, glycol or glycerin, but no more than about 70% of dextrose, mannitol, fructose, polyethylene glycol, glycol or glycerin. One of skill in the art could readily determine other highly hydrated excipients that would function similarly.

The inventors have recognized that in preparing liquid dosage forms, components necessary for dosage characteristics such as but not limited to palatability and suspension can be chosen in order to keep the concentration of diffusible counterions low and to choose components that are polyelectrolytes capable of further trapping the drug in the ion-exchange matrix drug complex.

Non-limiting examples of useful positively-charged polyelectrolytes include oligomers and polymers comprising the ammonium and/or tetralkylammonium salt forms of alkylaminoethyl(meth)acrylate, dimethylaminoethyl(eth)acrylate, aminoethyl(meth)acrylate, dimethylaminoethyl(meth)acrylate, dimethylaminopropyl(meth)acrylamide, vinyl pyridine, vinyl-N-ethylpyridine, vinylbenzyldimethylamine; or a positively charged hydrophillic colloid such as. e.g., chitosan; and any combination thereof. In a specific embodiment, the positively-charged polyelectrolyte is chitosan.

Non-limiting examples of useful negatively-charged polyelectrolytes include oligomers and polymers comprising the salt form of (meth)acrylic acid, (eth)acrylic acid, itaconic acid, maleic acid anhydride, vinylsulfonic acid, vinyl sulfuric acid, styrenesulfonic, vinylphenylsulfuric acid, alginate, xanthan gum, carboxymethyl cellulose, carboxymethyl-hydroxyethyl cellulose, dextran sulfate, hyaluronic acid, heparin, chondroitin sulfate, galacturonic acid, glutamic acid, gellan gum and any mixture thereof. In a specific embodiment, the negatively-charged polyelectrolyte is xanthan gum.

In one embodiment, the polyelectrolyte molecules do not further comprise diffusible counterions.

The inclusion of a like-charged, high molecular weight, polyelectrolyte in the dispersion medium, which is not capable of diffusing through the membrane, forces the lower molecular weight drug into the dispersed phase (through Donnan membrane effect) where it is confined by electrostatic force.

The liquid form controlled release drug composition can further comprise a dispersion additive selected from the group consisting of stabilizing agents, dispersing agents, and the like, provided the excipients do not adversely affect the intended operation of the invention.

The liquid form controlled release drug composition can further comprise excipients useful in oral liquid dose formulations such as, e.g., sweetening agents, flavoring agents, coloring agents, thickeners, and the like, provided the excipients do not adversely affect the intended operation of the invention.

The present invention contemplates the use of the dispersion additives and excipients discussed in the above embodiments that are also polyelectrolytes having a similar charge as the electrolytic drug. The invention also encompasses the use of such dispersion additives and excipients discussed in the above embodiments as the non-drug components included in the bead to reduce osmotic pressure or in the dispersion medium to impede drug dissolution prior to administration.

Advantages of the dosage form of the present invention is that the ion-trapping, osmotic control, and thermodynamic balancing mechanisms are generally applicable and inherently stable and the fact that effects can be implemented through the use of traditional and widely accepted pharmaceutical excipients one skilled in the art could utilize.

For illustration, the example of a cation-forming drug (propranolol) employing an alginate core bead will be used as a specific example for each stage, but the overall objective is to create polyelectrolyte(s) with a surface charge selected so as to confine oppositely charged drug molecules in the diffuse double layer (“DDL”) of the colloidal dispersed phase as depicted in FIG. 3. Typically, the dispersed phase will be aggregated in the form of an approximately spherical particle with a diameter in the 100 to 2000 um range. The size of the particles and/or swelling can be restricted through the use of cross-linking and/or cross-linking agents. In various embodiments, cross-linking can be accomplished through covalent bonding, e.g., AcDiSol is cross-linked carboxymethyl cellulose and cross-linked gelatin can be achieved via the exposure of gelatin to formaldehyde.

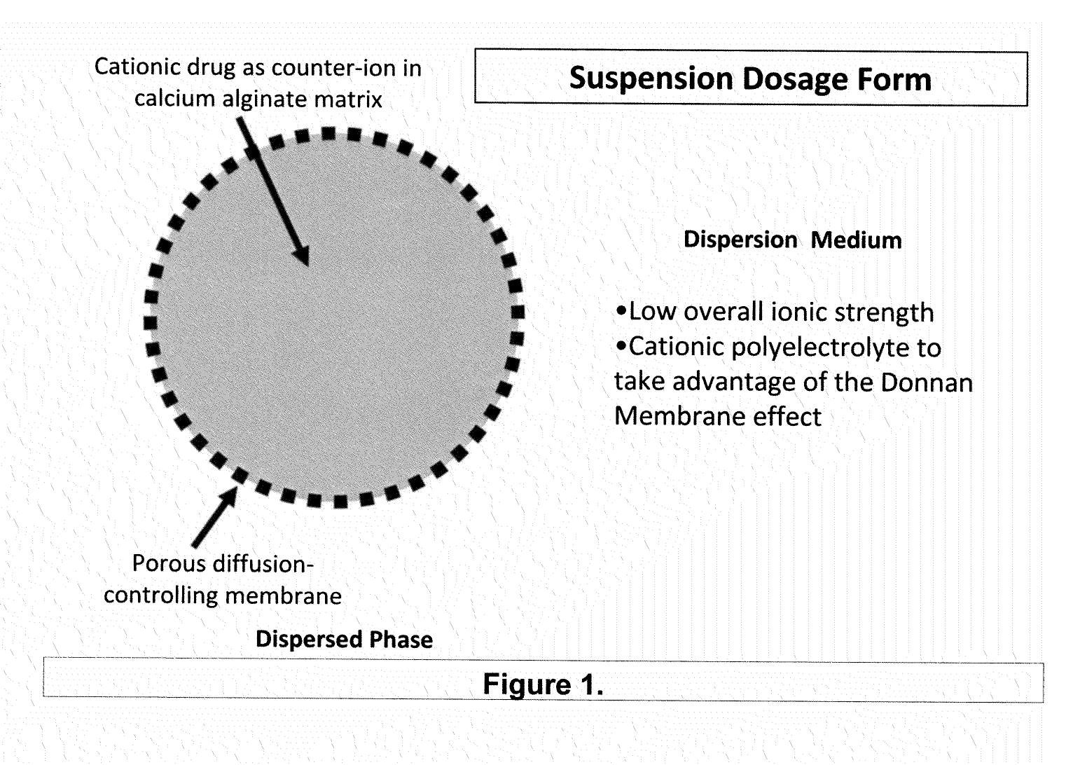

One embodiment of the invention is depicted in FIG. 1. An electrolytic or ionic form of a drug is confined in a dispersed phase of a suspension 1 comprising an ion exchange matrix with a charge opposite that of the drug 2. The dispersion medium 3 comprises a polyelectrolyte 4 with a surface charge the same as that of the drug. A thermodynamically stable trapping of water-soluble electrolytic drug in the dispersed phase 1 of the suspension 2 is achieved using electrostatic interactions, allowing a water-soluble drug to be confined in the dispersed phase 1 at high concentration without diffusing into the dispersion medium 3. A reservoir is created in which the drug will remain in the dispersed phase 1 until release is triggered by a high concentration of ions similar in charge to the drug in the dispersed phase. This triggering occurs when the drug is administered into physiological fluids with relatively high ionic strength. When the dispersed phase 1 comprises an optional diffusion-controlling membrane coating 5, the rate of release can be further controlled, because the rate of counterion exchange and electrolyte diffusion is inhibited.

Also by way of illustration, the exemplary drug albuterol will be used to demonstrate the applicability of a drug delivery system comprising drug-loaded beads manufactured from a physically stable aqueous gel that incorporates lactose. Typically such beads are then suspended in a dispersion medium containing sucrose. However, the overall objective is to create a stable aqueous gel by reacting any charged drug with an ion-exchange matrix having a charge opposite that of the drug in the presence of water to form beads that further comprise a soluble non-electrolyte capable of dissolving and diffusing out of the bead to relieve the osmotic pressure in the beads. Typically such beads are coated with a polymeric membrane that contributes to the control of drug release and the beads are suspended in a dispersion medium that includes a non-electrolytic excipient that is highly hydrated and capable of ensuring that the water activity outside the beads in the dispersion medium is less than the water activity inside the bead.

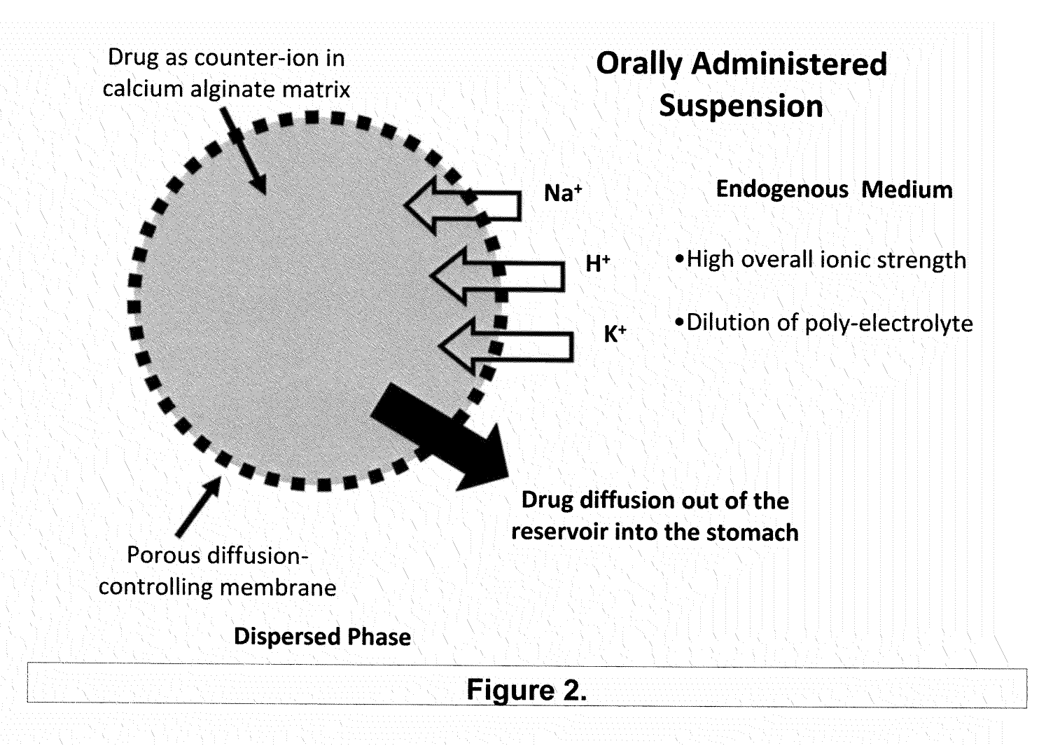

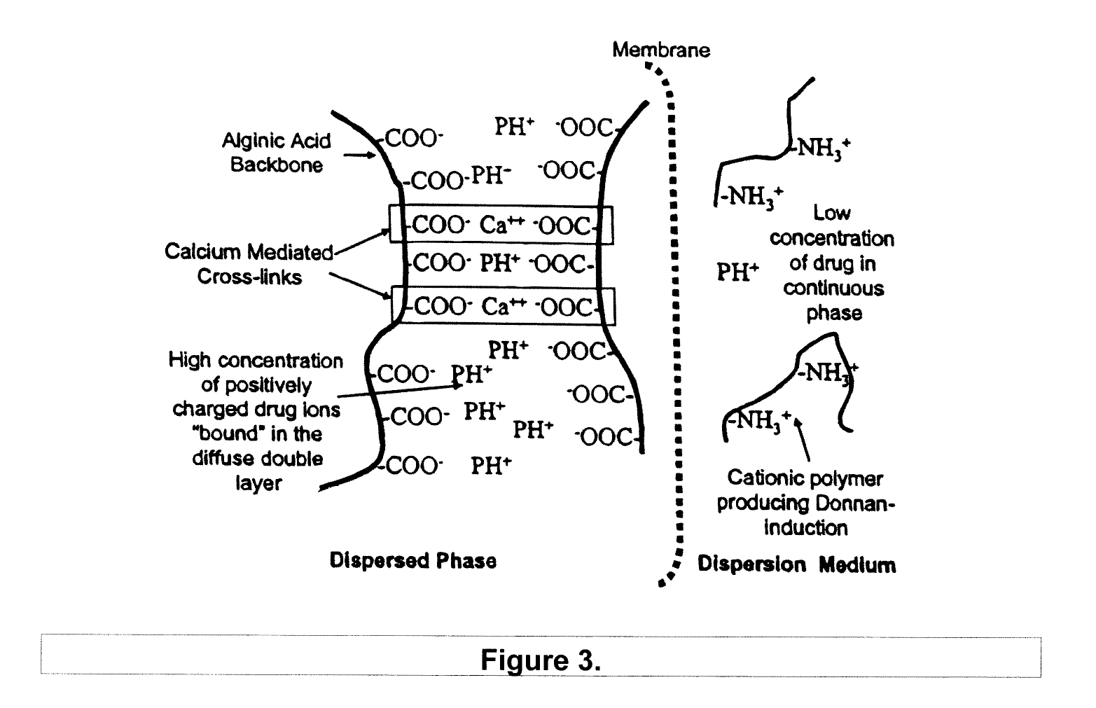

In the present system, the selection of a polyelectrolyte with a surface charge the same as the drug can serve to further depress free drug concentration in the dispersion medium. This occurs because of the Donnan membrane effect, where the transfer of the drug ion is inhibited by repulsion with the liked charged polyelectrolyte (see M. R. Franklin et al. Drug Absorption, Action, and Disposition in 57 Remington: The Science and Practice of Pharmacy 1118 (A. R. Gennaro, ed. 2000)) The fact that the polyelectrolyte has high molecular weight means that it can not diffuse through the diffusion controlling membrane on the drug-loaded bead. As a result, to maintain constant activity across the membrane, the diffusible drug is pushed into the bead. Thus, the membrane controls both the influx of swamping electrolyte and the efflux of drug. This mechanism is illustrated in FIG. 2. for a dispersed phase 1 comprising a calcium alginate matrix and a cationic electrolytic drug. After oral administration, the dispersion medium 3 is admixed with biological fluids having a high concentration of counter-cations such as sodium, potassium and proton. The counter-cations penetrate the diffusion-controlling membrane coating 5 and displace the electrolytic drug associated with the calcium alginate. The electrolytic drug then moves from an area of high concentration (the dispersed phase 1), crosses the diffusion-controlling membrane coating 5, and moves to an area of low drug concentration (the dispersion medium 2). Thus, the liquid dosage, thermodynamically stable, drug suspension possessing low free drug concentration in the dispersion medium, provides sustained drug release when administered to a patient. If chosen judiciously, the inclusion of a polyelectrolyte in the dispersion medium of a suspension can also serve the traditional purpose of increasing viscosity and slowing sedimentation.

The amount of polyelectrolyte used in the liquid form controlled release drug composition depends on a several factors such as but not limited to, e.g., the amount and type of electrolytic drug in the ion-exchange matrix drug complex; the type of ion-exchange matrix; the type of porous diffusion-control membrane, if any, used; the type of liquid used in the dispersion medium; and the charge density on the polyelectrolyte. In one embodiment, the polyelectrolyte content ranges from about 0.1 molar equivalents of ionic charge to about 10 molar equivalents of ionic charge per molar equivalent electrolyte drug. As used herein, the phrase “molar equivalents of ionic charge” refers to the number of salt sites having the same charge as the electrolytic drug. In another embodiment, the polyelectrolyte content ranges from about 0.5 molar equivalents of ionic charge to about 5 molar equivalents of ionic charge per molar equivalent electrolyte drug. In another embodiment, the polyelectrolyte content ranges from about 1 molar equivalents of ionic charge to about 3 molar equivalents of ionic charge per molar equivalent electrolyte drug.

The drugs useful in the embodiments of the present invention, and their recommended usage are known in the art and have been described in such literature as the Physician's Desk Reference (56^{th} ed., 2002). One skilled in the art could readily determine the electrolytic drugs that would be particularly suitable for the dosage forms contemplated by the present invention.

The electrolytic drugs useful in the invention include but are not limited to, e.g., cardiovascular drugs, beta-agonists, respiratory drugs, antidepressants, antipsychotics, sympathomimetic drugs, cholinomemetic drugs, adrenergic drugs, antimuscarinic and antispasmodic drugs, skeletal muscle relaxants, diuretic drugs, anti-migraine drugs, anesthetics, sedatives and hypnotics, antiepileptics, psychopharmacologic agents, analgesics, including opioid and non-opioid analgesics, antipyretics, CNS stimulants, antineoplastic and immunosuppressive drugs, antimicrobial drugs, antihistamines, anti-inflammatory drugs, antibiotics, decongestants, cough suppressants, expectorants, and the like.

Non-limiting examples of cationic-active agents useful in the present include the pharmaceutically acceptable salts of acetophenazine, albuterol, amitriptyline, benztropine, biperiden, bitolerol, bromodiphenhydramine, brompheniramine, buprenorphine, carbinoxamine, chlorcyclizine, chlorpheniramine, chlorphenoxamine, chlorpromazine, clemastine, clonidine, codeine, cyclobenzaprine, cyclizine, cyclobenzaprine, cyproheptadine, desipramine, dexbrompheniramine, dexchlorpheniramine, dextroamphetamine dibucaine, dextromethorphan, dicyclomine, diethylpropion, dihydroengotamine, diltiazem, diphemanil, diphenhydramine, doxepin, doxylamine, ephedrine, ergotamine, fluphenazine, haloperidol, hydrocodone, hydroxychloroquine, hydroxyzine, hyoscyamine, imipramine, isoproterenol, levopropoxyphene, lidocain, maprotiline, meclizine, mepenzolate, meperidine, mephentermine, mesoridazine, metaproterenol, methadone, methdilazine, methscopolamine, methysergide, metoprolol, morphine, nalorphine, nortriptyline, noscapine, nortriptylenepyrilamine, nylindrin, orphenadrine, papaverine, pentazocine, phendimetrazine, phentermine, phenylpropanolamine, phenmetrazine, phenelzine, pibuterol, procaine, prochlorperazine, promazine, propoxyphene, propanolol, protriptyline, pseudoephedrine, pyridostigmine, pyrilamine, quinidine, salmeterol, scopolamine, terbutaline, tetracaine, tranylcypromine, trihexyphenidyl, trimeprazine, tripelennamine, triprolidine and verapamil. In one embodiment, the cationic active agent is an analgesic selected from the group consisting of codeine and morphine. In another embodiment, the cationic active agent is an analgesic such as codeine or morphine. In a specific embodiment, the cationic active agent is chlorpheniramine, hydrocodone or pseudoephedrine.

Non-limiting examples of anionic active agents useful in the present invention include the pharmaceutically acceptable salts of acetylsalicylic acid, cromolyn, diclofenac, diclofenac, diflunisal, ethacrynic acid, fenoprofen, fentanyl, flurbiprofen, furosemide, gemfibrozil, ibuprofen, indomethacin, indoprofen, ketoprofen, naproxen, pentobarbital, phenyloin, salicylamide, salicylic acid, secobarbital, sulindac, thiopental, theophylline and valproate.

In one of the preferred embodiments, useful drugs include salt forms of the above mentioned electrolytic drugs. In one embodiment, a salt form of the drug may be maleate, hydrochloride or bitartrate.

In certain embodiments, useful drugs also include the neutral forms of the above mentioned electrolytic drugs which form ions upon association or reaction with the ion-exchange matrix.

In various embodiments, the ion-exchange matrix drug complex comprises ion-exchange matrix in an amount sufficient to convert the drug into ionic form. Optionally, the ion-exchange matrix drug complex comprises ion-exchange matrix in an amount more than sufficient to convert the drug into ionic form.

Also contemplated is the use of two or more electrolytic drugs in the dispersed phase of the liquid form controlled release compositions of the invention. In one embodiment of the invention, the dispersed phase comprises two, three, four or more drugs or active ingredients. In certain embodiments, the dispersed phase comprises three active ingredients.

It is also envisaged by the inventors that two or more drugs or active ingredients associate in a single particulate, pellet or bead. In one embodiment, two or more drugs or active ingredients associate in single particulate, pellet or bead in a liquid form controlled release drug composition of the invention.

In one of the embodiments, two, three or more drugs associate with the pharmaceutically acceptable ion-exchange matrix having a surface charge opposite that of the drugs. In one of the preferred embodiments, two, three or more drugs associate with the same pharmaceutically acceptable ion-exchange matrix having a surface charge opposite that of the drugs in a single particulate, pellet or bead. Surprisingly, inventors found that binding two or more drugs to the same ion-exchange matrix does not interfere with the controlled release of each drug in the composition, and provides an adequate rate of release of each drug. It is envisioned that cationic active ingredients associate with an ion-exchange matrix with a negative surface functionality, and anionic active ingredients associate with an ion-exchange matrix with a positive surface functionality. In another embodiment, the dispersed phase contains pharmaceutically acceptable salt forms of one or more drug or active ingredient. In yet another embodiment, the dispersed phase contains a pharmaceutically acceptable salt form of an ion-exchange matrix.

In certain embodiments, the compositions of the invention comprise two or more drugs in a single particulate, pellet or bed wherein there are no chemical or physical interactions between the two or more drugs in such particulate, pellet or bead, and acceptable stability characteristics and an adequate drug delivery profile are achieved with a single drug release rate-controlling coating.

If each of the two or more drugs are placed in separate beads, which are then mixed, it is possible that the mixture will not be perfectly homogenous, which will result in incorrect relative doses for the drugs. Non-homogeneity can occur either due to random fluctuations or due to different physical properties of the two or more beads in a mixture, such as a difference in weight and density. However, the above-described technology, wherein two or more drugs associate in a single particulate, pellet or bead, advantageously ensures homogeneity of the drug mixture and resulting dose uniformity of the respective drugs in the combination formulation, such that a given patient will receive the same amount of each of the two or more drugs. Further, binding of two or more drugs to the same ion exchange matrix provides an advantage of having only one drug-bound resin complex in the dispersed phase. Furthermore, such combining of two or more active ingredients into single particulate, pellet, or bead, may reduce the surface area of particulates, pellets, or beads present in an overall drug combination product, and may increase stability of the product and active ingredients.

Another advantage of the above-described technology of the present invention is that it achieves similar or the same release profile for all drugs bound to the same resin, and does not require different doses or frequency of dosing for each drug. If different resins are used, then the release profile of each drug may be affected by differences in a patient's diet or physiology, such that a given patient may receive too much of one drug, but too little of another. By contrast, advantageously, the above-described technology of the present invention allows to achieve bioequivalence for two or more drugs at the same time. Also, placing two or more drugs in a single bead with a single release technology, allows to achieve a drug release profile that will be more consistent across the patient population.

Yet another advantage of the above-described technology, wherein two or more drugs associate in a single particulate, pellet or bead, is that such technology makes it difficult to extract or separate out individual active ingredients of such drug combination. Specifically, binding of two or more active ingredients to the same ion-exchange matrix makes extraction or separation of any single active ingredient exceedingly difficult for an untrained individual. For example, if several drugs are bound to one ion-exchange matrix, it is not possible to partially isolate one drug from others on the basis of differences in the physical properties, such as densities, of individual one drug/one ion-exchange matrix complexes. Because of difficulty of isolation of any single active ingredient from products produced using the above-described technology of the present invention, such products will have a decreased potential for abuse or illegal use of any single ingredient in the product. Thus, the technology of the present invention enables manufacture of combination drug products that include active ingredients that can be abused (e.g., opioids, pseudophedrine, ephedrine), such that the resulting product has a decreased potential for drug abuse and diversion.

In yet another embodiment, two or more drugs associate with different pharmaceutically acceptable ion-exchange matrices having a surface charge opposite that of the respective drugs.

Still further contemplated is the use of one or more non-electrolytic drugs in the dispersion medium. In some embodiments, the dispersion medium comprises one, two, three, four or more non-electrolytic drugs. In certain embodiments, the dispersion medium comprises two or three non-electrolytic drugs.

In one of the preferred embodiments, the compositions of the invention are stable for a long period of time, i.e., for at least 1 month, for at least 3 months, for at least 6 months, or for at least or about one year, or for two years, or for three, four, five or more years. Specifically, the compositions of the invention maintain chemical, physical and microbiological stability for above-indicated periods of time. One of skill in the art would know how to assess chemical, physical and microbiological stability of a drug composition. Stability characteristics of a drug composition, e.g., physical, chemical and microbiological stability characteristics, determine how long a drug or an active ingredient can be stored in a bottle in a final composition ready for administration to a patient. In one of the embodiments, the compositions of the invention are stable at room temperature for at least 1 month, for at least 3 months, for at least 6 months, or for at least one year, or two years, three, four, five or more years. In such embodiments, the compositions of the present invention possess chemical, physical and microbiological stability for at least 1 month, for at least 3 months, for at least 6 months, for at least one year, or two years, or three, four, five or more years.

Chemical stability is manifested in structural integrity of drugs or active ingredients in the composition over time. Chemical stability may be assessed using chromatographic assays and/or potency measurements. Such assays detect the presence of a drug in a composition and presence or absence of degradation products. The drug is considered chemically stable at a time X, wherein X is a longer time period than zero (i.e., the time when a composition is manufactured), if at that time X 90-100% of the drug, which was present at time zero, is present and demonstrates adequate structural integrity characteristics, or the same or similar, or not significantly different structural characteristics in comparison to those expected at time zero. In one embodiment, the compositions of the present invention possess chemical stability for at least 1 month, for at least 3 months, for at least 6 months, or at least one year, or two years, or three, four, five or more years.

Physical stability of the compositions of the present invention is manifested in, e.g., integrity of a diffusion controlling membrane or a functional coating enveloping the dispersed phase, and its permeability. Physical stability may be assessed using a dissolution assay. A dissolution assay may be performed starting at a time zero (i.e., the time when a composition is manufactured) or at a time X, wherein X is any time above zero, such as, 1 week, 2 weeks, 3 weeks, 1 month, 3 months, 6 months, 1 year, 2 years, 3 years, 4 years, or 5 years. The dissolution testing shows the amount of drug released once the composition is placed in a chemical environment which is equivalent to the environment in a patient's gastrointestinal tract. The dissolution testing reflects the rate of release of a drug upon its administration to a patient. The dissolution or release profile, i.e. the amount of drug released over time at each time point measured (after its placing in an appropriate chemical environment or its administration to a patient) and the rate of release of a drug, is indicative of the physical stability of a drug composition. A physically stable drug composition at a certain time X would manifest the same, similar, or at least not significantly different dissolution or release profile assessed using a dissolution assay as would be expected from a drug composition tested at a time zero, i.e., when the assay is performed immediately after the drug is manufactured. Also, a physically stable drug composition would have a certain expected amount of a drug released at the first time point of the assay (i.e., at the start of the assay). More specifically, at the first time point of the assay, a physically stable composition, which does not have an immediate release portion, would exhibit no drug release, or very low level of drug release, i.e., a release of less than 10%, or less than 5%, or less than 1% of the drug from the extended release phase. Further, a physically stable drug composition would have a certain expected rate of drug release, i.e., certain amount of drug released at each subsequent time point upon placing it in an appropriate chemical environment or its administration to a patient. In one embodiment, the compositions of the present invention possess physical stability for at least 1 month, for at least 3 months, for at least 6 months, or at least one year, or two years, or three, four, five or more years. In such embodiment, a drug composition of the invention may be stored in a bottle for at least 1 month, for at least 3 months, for at least 6 months, or at least one year, or two years, or three, four, five or more year, while maintaining its physical stability.

In certain embodiments, the compositions of the present invention also maintain microbiological stability over time. Microbiological stability of a drug composition reflects absence of contamination with microorganisms of such composition. In one embodiment, the compositions of the present invention possess microbiological stability for at least 1 month, for at least 3 months, for at least 6 months, or at least one year, or two years, or three, four, five or more years.

Due to the stability characteristics of the liquid form controlled release compositions of the present invention, such compositions have a long shelf life, i.e., shelf life of one year or more. It is envisaged that the compositions of the present invention have a storage shelf life at room temperature conditions of at least 1 month, of at least 3 months, of at least 6 months, or at least about one year, or about two years, or three, four, five or more years, during which time the stability and drug release profile characteristics of such formulations are maintained. In other embodiments, the sustained release formulation is physically and chemically stable for more than 1 month, more than 3 months, more than 6 months, or more than 1 year, or more than about 2 years, or more than about 3 years, or more than 4 years or more than 5 years.

In another embodiment, the present invention provides a combination formulation comprising one or more drugs, for example three drugs, and comprising an extended release and an immediate release portion which, when administered to a patient, achieves bioequivalence to immediate release product/s containing these drug/s. One advantage is that he formulations of the present invention may achieve bioequivalence for two or more drugs at the same time. In one embodiment, one or more drugs comprise an antihistamine, an antitussive, and a decongestant.

In certain embodiment, the present invention relates to an oral extended release drug composition comprising a first portion and a second portion, wherein

the first portion comprises an antihistamine, an antitussive, and optionally a decongestant as active ingredients in an immediate release form,

the second portion comprises a particulate, pellet, or bead that comprises the antihistamine, the antitussive, and the decongestant as three active ingredients in an extended release form,

administration of a single dose of the oral drug composition to a patient provides serum levels of the three active ingredients over a time period of at least 8 hours that are bioequivalent to serum levels achieved upon administration of an appropriate number of doses, over the same time period, of FDA-approved immediate release reference listed drug (IR RLD) compositions comprised of the active ingredients, and

the appropriate number of doses corresponds to a number of doses recommended in one or more FDA-approved labels for administration of the IR RLD compositions over the same time period.

Further, in one such embodiment, the antitussive is a narcotic antitussive. In another such embodiment, the first portion does not comprise the decongestant. In another such embodiment, the drug composition is in an oral liquid suspension form. In one such embodiment, the particulate, pellet or bead further comprises a coating. In another such embodiment, the antihistamine, the antitussive and the decongestant associate in a single particulate, pellet or bead. In yet another such embodiment, the particulate, pellet or bead further comprises a pharmaceutically acceptable ion-exchange matrix, wherein the antihistamine, the antitussive and the decongestant associate with the ion-exchange matrix. One such embodiment is further comprising an excipient selected from the group consisting of sweetening agents, flavoring agents, coloring agents, and any combination thereof. Another such embodiment is further comprising an additive selected from the group consisting of stabilizing agents, dispersion agents, and any combination thereof.

In yet another embodiment, the present invention relates to an oral extended release drug composition comprising a first portion and a second portion, wherein

the first portion comprises an antihistamine, an antitussive, and optionally a decongestant, as active ingredients in an immediate release form,

the second portion is a particulate, pellet or bead that comprises the antihistamine, the antitussive, and the decongestant as active ingredients in an extended release form,

administration of a sufficient number of doses of the drug composition to a patient to achieve steady-state serum levels of the three active ingredients over a time period of greater than 24 hours yields serum levels of the active ingredients that are bioequivalent to serum levels achieved upon administration of an appropriate number of doses, over the same time period, of one or more FDA-approved immediate release drug compositions comprised of the active ingredients, and

the appropriate number of doses corresponds to a number of doses recommended in one or more FDA-approved labels for the administration of the one or more FDA-approved immediate release drug compositions over the same time period.

In certain embodiments, the present invention relates to a method for achieving in a mammal serum levels of an antihistamine, an antitussive and a decongestant over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:

(a) administering to the mammal an oral extended release drug composition comprising a first portion and a second portion, wherein the first portion comprises the antihistamine, the antitussive and optionally the decongestant as active ingredients in an immediate release form, and wherein the second portion comprises a particulate, pellet, or bead that comprises the antihistamine, antitussive and the decongestant as active ingredients in an extended release form, and

(b) achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

In other embodiments, the present invention relates to a method for achieving in a mammal steady-state serum levels of an antihistamine, an antitussive and a decongestant upon administration of an oral extended release (ER) drug composition, wherein the serum levels are bioequivalent to serum levels achieved upon administration of one or more immediate release (IR) compositions comprising active ingredients and inactive ingredients, wherein said active ingredients consist of an antihistamine (e.g., chlorpheniramine), an antitussive (e.g., hydrocodone) and pseudoephedrine (e.g., pseudoephedrine) to the same mammal, wherein the method comprises:

administering to the mammal an oral ER drug composition comprising a first portion and a second portion, wherein the first portion comprises the antihistamine, the antitussive and the decongestant as active ingredients in an immediate release form, and wherein the second portion comprises a particulate, pellet or bead that comprises the antihistamine, the antitussive and the decongestant as active ingredients in an extended release form, and

wherein administration of a sufficient number of doses of the oral ER drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,

wherein the appropriate number of doses of the one or more FDA-approved IR drug compositions corresponds to a number of doses recommended in one or more FDA-approved labels for the administration of the one or more FDA-approved IR drug compositions over the same time period, and

wherein the appropriate number of doses of the one or more FDA-approved IR drug compositions is greater than the sufficient number of doses of the oral ER drug composition.

### 5.3.2 Methods for Making the Liquid Dosage Forms

The present invention also relates to methods for making the liquid sustained release drug formulations contemplated by the present invention.

In one embodiment, the present invention relates to a method for making a liquid sustained release drug formulation, comprising:

(a) providing an ion-exchange resin matrix;

(b) allowing the drug to associate with the ion-exchange matrix to form an ion-exchange matrix drug complex;

(c) drying the ion-exchange matrix drug complex; and

(d) combining the ion-exchange matrix drug complex with a dispersion medium.

In one embodiment, the dispersion medium of step (d) is substantially free of diffusible counterions.

In another embodiment, the dispersion medium of step (d) further comprises a polyelectrolyte.

In another embodiment, step (d) further comprises adding a polyelectrolyte having the same charge as the electrolyte drug to the dispersion medium.

In another embodiment, step (c) further comprises coating the ion-exchange matrix drug complex with a porous diffusion-controlling membrane.

Each of the above steps (a)-(d) influences the nature and performance of the finished product. In some situations, the drug loading step may be conducted after the core beads have been created.

In one embodiment, the method involves the formation of an aqueous gel.

In another embodiment, the method involves incorporation of a low molecular weight, non-electrolytic, soluble excipient in the formation of the dispersed phase in steps (a) and (b).

In another embodiment, the dispersion medium of step (d) further comprises adding a highly hydrated excipient capable of ensuring greater water activity outside the dispersed phase in the dispersion medium than inside the dispersed phase.

In one embodiment, the present invention relates to a method for making a liquid sustained release drug formulation, comprising:

(a) providing an ion-exchange resin matrix;

(b) allowing the drug to associate with the ion-exchange matrix in the presence of water to form an ion-exchange matrix drug complex, wherein the ion-exchange matrix drug complex is an aqueous gel;

(c) drying the ion-exchange matrix drug complex; and

(d) combining the ion-exchange matrix drug complex with a dispersion medium.

In one embodiment, the dispersion medium of step (d) is substantially free of diffusible counterions.

In another embodiment, the dispersion medium of step (d) further comprises a polyelectrolyte.

In another embodiment, step (d) further comprises adding a polyelectrolyte having the same charge as the electrolyte drug to the dispersion medium.

In another embodiment, step (c) further comprises coating the ion-exchange matrix drug complex with a porous diffusion-controlling membrane.

Each of the above steps (a)-(d) influences the nature and performance of the finished product. In some situations, the drug loading step may be conducted after the core beads have been created.

In another embodiment, the method involves incorporation of a low molecular weight, non-electrolytic, soluble excipient in the formation of the dispersed phase in steps (a) and (b).

In a specific embodiment, the base form of albuterol is allowed to interact with alginic acid in the presence of water, surprisingly forming a physically stable aqueous gel that can be used in forming drug-loaded beads. In one embodiment, the drug-loaded beads further comprise lactose. In other embodiments, the beads can optionally comprise stearic acid or other excipients that can facilitate extrusion. In certain specific embodiments, the resulting beads are coated with EUGRAGIT® and suspended in a dispersion medium that comprises water and sucrose. In one specific embodiment, the dispersion medium comprises Syrup NF. The dispersion medium can also further comprise preservatives and other non-active additives. In such embodiments, the resulting liquid sustained release product is capable of maintaining physical stability in a bottle and capable of achieving controlled release of drug product when administered to a patient.

In another embodiment, the dispersion medium of step (d) further comprises adding a highly hydrated excipient capable of ensuring greater water activity outside the dispersed phase in the dispersion medium than inside the dispersed phase.

In one embodiment, the invention relates to a method for preparing a liquid form controlled release drug composition, comprising:

(a) allowing a water-soluble electrolytic drug to associate with an ion-exchange matrix to form an ion-exchange matrix drug complex; and

(b) dispersing the ion-exchange matrix drug complex into a dispersion media comprising a polyelectrolyte; wherein

the surface of the ion-exchange matrix has a charge opposite that of the electrolytic drug; and

the polyelectrolyte has the same charge as that of the electrolytic drug.

In another embodiment, the present invention relates to a method for preparing a liquid form controlled release drug composition, comprising:

(a) allowing the acid form of an acid-functional ion-exchange matrix to associate with the base form of an amine-based drug to form an ion-exchange matrix drug complex; and

(b) dispersing the ion-exchange matrix drug complex into a dispersion media comprising a polyelectrolyte; wherein
the polyelectrolyte has a positive charge.

The invention further contemplates methods that encompass the formation of uncomplicated aqueous gels comprising drug and ion-exchange matrix. In one embodiment, such aqueous gels are substantially free of spectator ions, and are spontaneously formed by reacting and hydrating a drug and an ion-exchange matrix having a charge opposite that of the drug. In certain embodiments, manipulating drug and ion-exchange matrix in solid forms having low to limited aqueous solubility can allow formation of a stable colloidal dispersion which ultimately allows a highly water soluble drug to be formulated into a liquid sustained release drug delivery system. In particular embodiments, the resulting aqueous gel is viscous with adhesive characteristics suitable for granulation or extrusion/spheronization. In other particular embodiments, the drug is distributed with content uniformity within the aqueous gel. In yet other particular embodiments, the electrostatic interaction between the drug and ion-exchange matrix and/or permeable membrane influences the rate and extent of drug release. It is also contemplated that the aqueous gel can be used in an aqueous coating process to layer drug on a sugar sphere, forming a bead. The gel can also be dried to produce hydrophilic drug complex that can be easily reconstituted with rapid dissolution. The methods contemplated by the instant invention can be easily scaled up for large-scale manufacturing. As such, the instant invention also encompasses novel drug-loaded beads formed from an aqueous gel, having excellent content uniformity and high hydrophilicity, readily produced in large quantities.

In another embodiment, the present invention relates to a method for preparing a liquid form controlled release drug composition, comprising:

(a) allowing the acid form of an acid-functional ion-exchange matrix to associate with the base form of an amine-based drug in the presence of water to form an ion-exchange matrix drug complex, wherein the ion-exchange matrix drug complex is an aqueous gel;

(b) forming beads using the aqueous gel;

(c) coating the beads with a membrane; and

(d) dispersing the beads into a dispersion media

In certain embodiments, step (a) of the above method further includes the addition of a non-electrolytic soluble component having a molecular weight low enough to diffuse through the membrane. In one embodiment, the component is lactose.

In certain embodiments, the dispersion media of step (d) further includes the addition of a highly hydrated excipient capable of associating with water such that the water activity outside the beads in the dispersion media will be less than the water activity inside the beads.

An advantage of preparing the ion-exchange matrix drug complexes as aqueous gels is that spectator ions are absent from the complexes. A formulation based on an aqueous gel can comprise as few as one additional excipient and water. The resulting product formed from the aqueous gel is very hydrophilic with excellent content uniformity. Moreover, electrical neutrality constraints allow drug to be trapped by the ion-exchange matrix until there are molecules available to exchange with the drug, leaving the drug free to diffuse out of the bead.

In a specific embodiment, the dispersion media of step (d) is Syrup NF.

In another embodiment, the present invention relates to a method for preparing a liquid form controlled release drug composition, comprising:

(a) allowing the base form of an amine-functional ion-exchange matrix to associate with the acid form of an acid-based drug in the presence of water to form an ion-exchange matrix drug complex, wherein the ion-exchange matrix drug complex is an aqueous gel;

(b) forming beads using the aqueous gel;

(c) coating the beads with a membrane; and

(d) dispersing the beads into a dispersion media.

In certain embodiments, step (a) of the above method further includes the addition of a non-electrolytic soluble component having a molecular weight low enough to diffuse through the membrane. In one embodiment, the component is lactose.

In certain embodiments, the dispersion media of step (d) further includes the addition of a highly hydrated excipient capable of associating with water such that the water activity outside the beads in the dispersion media will be less than the water activity inside the beads.

In a specific embodiment, the dispersion media of step (d) is Syrup NF.

It will be understood that when the ion form of a drug is used to prepare the ion-exchange matrix drug complex, the drug is associated with one or more counterions. Illustrative counter-cations include, but are not limited sodium, potassium, and lithium; calcium, magnesium; aluminum and zinc or other similar metals; ammonia and organic amines, such as unsubstituted or hydroxy-substituted mono-, di- or trialkylamines; dicyclohexylamine; tributyl amine; pyridine; N-methyl-N-ethylamine; diethylamine; triethylamine; mono-, bis- or tris-(2-hydroxy-lower alkyl amines), such as mono-, bis- or tris-(2-hydroxyethyl)amine, 2-hydroxy-tert-butylamine or tris-(hydroxymethyl)methylamine, N,N-di-lower alkyl-N-(hydroxy lower alkyl)-amines, such as N,N-dimethyl-N-(2-hydroxyethyl)amine or tri-(2-hydroxyethyl)amine; N-methyl-D-glucamine; and amino acids such as arginine, lysine and the like. Illustrative counter-anions include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)).

The counterion associated with the ion-form of the drug may be the same or different from the diffusive counterion.

In another embodiment, the present invention relates to a method for preparing a liquid form controlled release drug composition, comprising:

(a) allowing the base form of an amine-functional ion-exchange matrix to associate with the acid form of acid-based drug to form an ion-exchange matrix drug complex; and

(b) dispersing the ion-exchange matrix drug complex into a dispersion media comprising a polyelectrolyte; wherein

the polyelectrolyte has a negative charge.

Non-limiting examples of useful ion-exchange matrix matrices include those described in Section 5.2 above. In another embodiment, the ion-exchange matrix is formed into a bead using standard forming methods including, but not limited to, granulation, extrusion and/or spheronization.

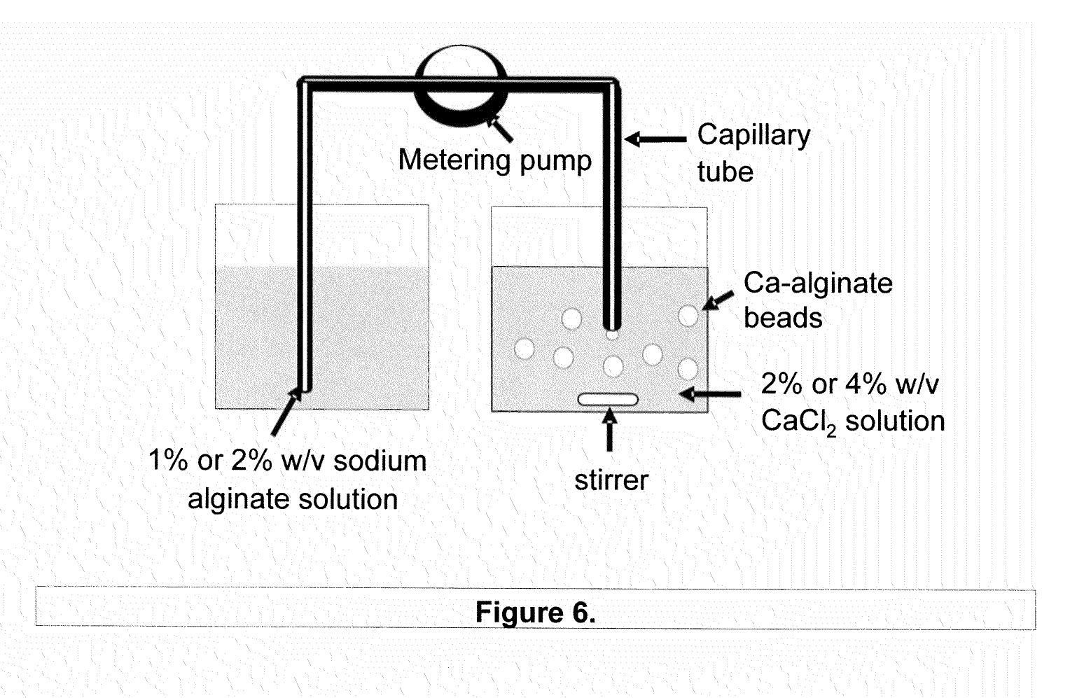

In certain embodiments, the ion-exchange matrix is a hydrophilic colloid formed through methods including, but not limited to coagulation precipitation of a hydrated liquid colloid. In another embodiment, the ion-exchange matrix is a cross-liked hydrophilic colloid. For example, a calcium cross-linked alginate can be formed by adding a 1 or 2% dispersion of sodium alginate drop-wise into a 2 or 4% solution of calcium chloride as shown in FIG. 6. After drying, the calcium cross-linked alginate can be coated with a porous diffusion-controlling membrane. The structure of the resultant calcium alginate drug complex is shown in FIG. 3. The alginic backbones 6 are cross-linked by Ca²⁺ cations 7 through —OC(O)-alginic pendant groups 8. Because of the ability of the Ca²⁺ cations 7 to crosslink alginate 6, a solid matrix is formed, which can be formulated into beads. These beads can be “harvested’ by filtration, and washed with distilled water to remove excess calcium chloride. The resulting beads have structure because of the cross-linking, and the negative carboxyl groups 9 that are not involved in cross links are neutralized by sodium counterions present in the diffuse double layer 10.

In certain embodiments, the ion-exchange matrix is first activated so that a suitable exchangeable ion is present on the surface of the ion-exchange matrix prior to loading with the electrolytic drug. For a cation-exchange matrix, the activation step typically involves treatment with dilute aqueous base such as, e.g., aqueous NaOH. For an anion-exchange matrix, the activation step involves treatment with dilute acid such as, e.g., aqueous HCl.

In certain embodiments, an ion-exchange matrix is allowed to interact with a drug having a charge opposite that of the ion-exchange matrix, in the presence of water, spontaneously and reversibly forming an aqueous gel, substantially free of spectator ions. The resulting gel is typically added to microcrystalline cellulose and other excipients into a low shear planetary mixer and the resulting wet mass is extruded, spheronized and dried using methods known in the art.

Drug loading may be accomplished by methods known in the art such as, e.g., ion displacement with an electrolytic drug in an aqueous or sufficiently polar non-aqueous dispersion; allowing an acid-functional ion-exchange matrix to associate with the base form of an amine-based drug to form an ion-exchange matrix drug complex; and allowing a free-base-functional ion-exchange matrix to associate with the acid form of a drug to form an ion-exchange matrix drug complex.

By way of illustration, the aforementioned calcium cross-linked alginate beads can be contacted with a concentrated solution of a drug such as propranolol hydrochloride. Because of the concentration gradient, the protonated propranolol cations will diffuse into the beads and displace the sodium counterions. By routine experimentation, one of ordinary skill in the art can determine through specific binding studies the affinity and capacity of the ion-exchange matrix and thereby control the drug loading step. After loading, the calcium cross-linked alginate beads can be harvested by filtration and washed with distilled water to remove unbound propranolol hydrochloride and sodium chloride.

Also by way of illustration, the aforementioned alginic acid can be combined with the base form of a drug such as albuterol in the presence of water. The components will spontaneously and reversibly form a hetergeneous aqueous gel. One of ordinary skill in the art can readily determine the ion-exchange matrix and drug characteristics that allow formation of an aqueous gel substantially free of spectator ions. The resulting gel can be granulated and/or spheronized and/or extruded and the resulting beads dried, and optionally coated. In specific embodiments, excipients such as lactose are incorporated into the beads.

Methods for contacting or loading the ion-exchange matrix with the electrolytic drug include, e.g., bulk mixing a solution phase of the electrolytic drug and the ion-exchange matrix; allowing the ion-exchange material to fall through a column of drug-containing solution; and circulating a solution phase of the electrolytic drug through a bed of the ion-exchange matrix. Changes in drug concentration in the liquid phase can be used to quantitatively monitor the progress of the drug loading.

As noted above, the present invention relates to methods for contacting or loading an acid-functional ion-exchange matrix with the free-base form of an amine-based drug, or a free-base-functional ion-exchange matrix with the acid form of a drug. An advantage of preparing the ion-exchange matrix drug complexes with the non-ionic form of the drugs is that non-aqueous solvents can be employed such as, e.g., methanol, ethanol, propanol, methylene chloride, glycols, and the like, provided that the solvent does not adversely interact with the ion-exchange matrix and the optional porous diffusion-controlling membrane.

Once the contacting step is complete, the solution phase can, optionally, be replenished with additional drug and used again in another contacting step. In another embodiment, the counterions present in the solution phase are removed by contacting the liquid phase with a size selective resin such as, e.g., those used for size exclusion chromatography or size exclusion filtration. The purified liquid phase can then be reused as described above.

As noted above, the ion-exchange matrix drug complexes are dried, and optionally coated with a diffusion-controlling membrane. Drying can be performed using traditional drying equipment known in the art. A fluid bed system is particularly suitable since the beads can be dried and coated in the same unit.

The optional diffusion-controlling membrane is applied to the ion-exchange matrix drug complex in an amount sufficient to control the release of the electrolytic drug from the dispersed phase.

Methods for coating solid form dosages are known in the art (see S. C. Porter, Coating of Pharmaceutical Dosage Forms in 57 Remington: The Science and Practice of Pharmacy 894-902 (A. R. Gennaro, ed. 2000, the entire contents of which are incorporated herein by reference). Coating methods useful in the present invention include those using fluidized bed technology including top spray, bottom spray and tangential spray; and pan coating.

In one embodiment, the ion-exchange matrix drug complex is spray coated with a spray comprising a diffusion-controlling membrane. In another embodiment, the ion-exchange matrix drug complex is spray coated with a spray comprising a diffusion-controlling membrane and an solvent. Non-limiting examples of solvents useful for spraying diffusion-controlling membrane include water; organic solvents such as, e.g., methanol, ethanol, propanol and dichloromethane; and combinations thereof. The coating may be applied as a solution or suspension.

In another embodiment, the invention relates to a solid phase of the liquid form controlled release drug composition comprising an ion-exchange matrix drug complex as described above; optionally, a diffusion-controlling membrane; and a polyelectrolyte. Such solid can be prepared by, e.g., allowing the liquid component of the dispersion medium of the liquid form controlled release drug composition to evaporate under reduced pressure.

The solid phase of the liquid form controlled release drug composition can be dispersed in a pharmaceutically acceptable liquid prior to use. Non-limiting examples of pharmaceutically acceptable liquids include deionized water; non-aqueous liquids such as, e.g., propylene glycol, glycerin, sorbitol solution and the like, which do not interfere with the intended operation of the invention; and any mixture thereof. In some of the preferred embodiments, the liquid further includes a highly hydrated excipient such as sucrose. As such, in one embodiment, the liquid is Syrup NF.

The present invention also relates to methods of forming an ion-exchange matrix drug complex using spray-coating or fluidized bed technology. In one embodiments, the invention relates to a method for forming a multilayer calcium alginate drug precursor, comprising:

(a) providing a seed;

(b) allowing a cation-forming electrolytic drug to be deposited on the seed to form an electrolytic drug layer;

(c) allowing a base to be deposited on the electrolytic drug layer to form a base layer;

(d) allowing alginic acid to be deposited on the base layer to form an alginic acid layer; and

(e) allowing a porous-diffusion controlling membrane to be deposited on the alginic acid layer to form the multilayer calcium alginate drug precursor.

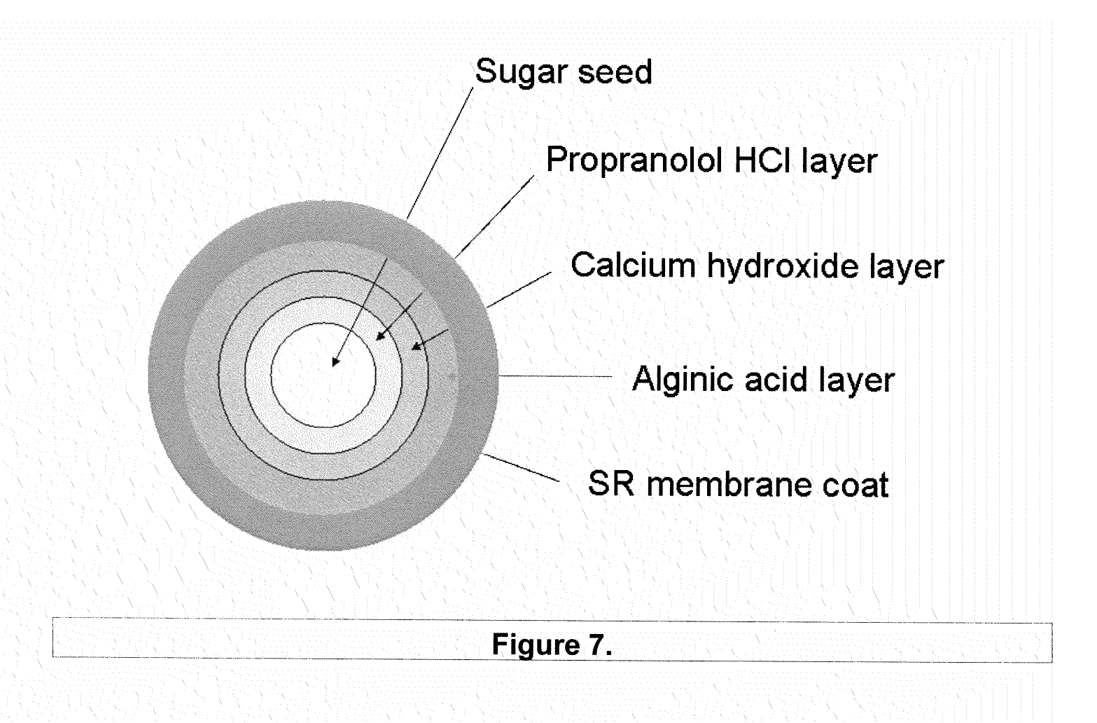

The above-described embodiment is depicted in FIG. 7.

In another embodiment, the method of step (e) further comprises admixing the calcium alginate drug precursor with water that is substantially free of diffusible counterions to form a calcium alginate drug complex.

In another embodiment, the method of step (e) further comprises dispersing the calcium alginate drug complex into a pharmaceutically acceptable liquid to provide a dispersion comprising a pharmaceutically effective concentration of the calcium alginate drug complex. In one embodiment, the dispersion liquid comprises a positively charged polyelectrolyte.

In another embodiment, the method of step (e) further comprises dispersing the calcium alginate drug complex into a pharmaceutically acceptable liquid; and adding a positively charge polyelectrolyte to the dispersion liquid.

In one embodiment, the seed is sugar.

Without being limited by theory, in the above embodiment, Applicants believe that the multilayer calcium alginate drug precursor forms a calcium alginate drug complex by the following process: The multilayer calcium alginate drug precursor, which is typically a dry bead, is immersed in an aqueous environment. Water diffuses into the bead, controlled to some extent by the diffusion-controlling membrane layer. Alginic acid hydrates as water penetrates the diffusion-controlling membrane. As the alginic acid continues to hydrate, water reaches the calcium hydroxide layer, the pH of the “wetted” phases is elevated, and calcium ions begin to diffuse into the alginic acid layer the calcium cross-linking begins to occur. The resultant calcium-alginate gel continues to react with base and cross-link, and water reaches the water-soluble cationic drug. The cationic drug dissolves is in the water, diffuses into the alginic acid layer, and is trapped in the calcium alginate matrix.

An advantage of the above-described embodiment is that all the electrolytic drug remains affixed to the calcium alginate drug precursor.

The concentration of ion-exchange matrix resin drug complex in the liquid form controlled release drug composition can vary over a wide range depending, e.g., on the particular drug, the content of drug in the of ion-exchange matrix resin drug complex; the condition or symptom to be treated; and the age of the patient. In one embodiment, the concentration of ion-exchange matrix resin drug complex in the liquid form controlled release drug composition ranges from about 5% to about 90% by weight based on the total weight of the liquid form controlled release drug composition; in the another embodiment, the weight of ion-exchange matrix resin drug complex ranges from about 10% to about 50% based on the based on the total weight of the liquid form controlled release drug composition; and in the another embodiment, the weight of ion-exchange matrix resin drug complex ranges from about 20% to about 40% based on the based on the total weight of the liquid form controlled release drug composition.

In yet another preferred embodiment, the method of making the liquid dosage forms of the invention does not involve formation of an aqueous gel comprising a drug and an ion-exchange matrix. Specifically, in such embodiment, the step of preparing the dispersed phase of the liquid dosage forms of the invention does not involve formation of an uncomplicated aqueous gel comprising a drug and an ion-exchange matrix. In another embodiment, the step of preparing the dispersed phase does not involve the step of conventional drug loading of the core ion-exchange beads.

In one of the preferred embodiments, the method of preparing the dispersed phase comprises mixing of a drug in a powder form and an ion-exchange matrix in a powder form. In such embodiment, salt forms of the drug and the ion-exchange matrix may be used. The powder blending method of preparing the dispersed phase of the present invention is cost and time-effective as compared to conventional prior art methods.

In one embodiment, the ion exchange matrix is sodium alginate. The drugs and pharmaceutically acceptable salt forms of such drugs that may be used in the liquid dosage of the invention have been described elsewhere in the specification. In one specific but non-limiting embodiment, powders of chlorpheniramine maleate, pseudoephedrine hydrochloride and hydrocodone bitartrate alone or in combination may be mixed with sodium alginate powders. In another embodiment, lactose, microcrystalline cellulose and/or other excipients may be added to the mixture of drug and ion-exchange powders.

In another embodiment, subsequent to the drug and ion-exchange powder blending, wet massing is continued with the addition of water, followed by extrusion and spheronization. The resulting core beads or pellets containing drug and ion-exchange matrix may be dried in a fluid bed dryer. In one of the preferred embodiments, each bead or pellet comprises several active ingredients associated with the same ion-exchange matrix. For example, each bead or pellet may comprise chlorpheniramine maleate, pseudoephedrine hydrochloride and hydrocodone bitartrate associated with sodium alginate powders.

In certain embodiments, the resulting beads are coated with EUDRAGIT® in the presence of triethyl citrate and talc in a fluid bed processor. Then, the coated beads are blended with talc and cured in an oven. In one embodiment, coated beads are cured for 2 h, 4 h, 8 h, 16 h, 24 h, or 48 h. In certain embodiments, coated beads are cured from about 16 hours to about 24 hours. The curing time of the coated beads may have an effect on a rate of release of the drug from the coated beads.

The coated beads are suspended in a dispersion medium that comprises salt forms of drugs, water and sucrose. In one specific but non-limiting embodiment, the dispersion medium comprises chlorpheniramine maleate and hydrocodone bitartrate. In another embodiment, the dispersion medium also comprises pseudoephedrine hydrochloride. The dispersion medium can also further comprise preservatives, taste masking agents and other non-active additives. In such embodiments, the resulting liquid sustained release product is capable of maintaining physical and chemical stability in a bottle, and capable of achieving controlled release of drug product when administered to a patient.

In one embodiment, the present invention relates to a method for preparing a liquid form controlled release drug composition, comprising:

(a) preparing the dispersed phase, which comprises preparing particulates, pellets or beads, wherein two or more active ingredients associate in a single particulate, pellet or bead;

(b) preparing the dispersion medium, wherein the dispersion medium comprises two or more active ingredients;

(c) coating the particulates, pellets or beads with a membrane coating; and

(d) dispersing the beads into a dispersion medium.

In one embodiment of the above-described method, the step of preparing the dispersed phase further comprises associating the two or more active ingredients with a pharmaceutically acceptable ion-exchange matrix. In another embodiment, the step of preparing the dispersed phase further comprises preparing particulates, pellets or beads comprising two or more active ingredients and a pharmaceutically acceptable ion-exchange matrix, wherein the two or more active ingredients bind to the ion-exchange matrix in a single particulate, pellet or bead. In another embodiment, the step of preparing the dispersed phase further comprises blending of the active ingredients and ion exchange matrix powders. In yet another embodiment, the step of preparing the dispersed phase further comprises wet granulation, extrusion and spheronization of the active ingredients and ion exchange matrix powders. In one embodiment, salt forms of the active ingredients and the ion exchange matrix are used.

In another embodiment, the present invention relates to a method for making the liquid oral extended release compositions of the invention, comprising

(a) preparing the immediate release portion, wherein the immediate release portion comprises an antihistamine, an antitussive, and optionally a decongestant as active ingredients;

(b) preparing the extended release portion, which comprises preparing particulates, pellets or beads comprising an antihistamine, an antitussive, and a decongestant as active ingredients;

(c) coating the particulates, pellets or beads with a membrane coating; and

(d) combining the extended release portion with the immediate release portion.

One embodiment of the above-described method further comprises preparing particulates, pellets or beads, wherein two or more active ingredients associate in a single particulate, pellet or bead. In another embodiment, the method further comprises the step of associating the two or more active ingredients with a pharmaceutically acceptable ion-exchange matrix.

### 5.3.3 Methods for Administering the Liquid Dosage Forms

While not restricted in their utility, the liquid sustained release dosage forms of the invention are useful for oral administration to a patient in need thereof.

It is another object of the invention to provide sustained release liquid dosage forms, suitable for once-a-day or twice-a-day administration of highly water soluble electrolytic drugs. In particular, the invention encompasses providing sustained release liquid dosage forms, capable of drug release over the span of about 24 hours, up to 48 hours.

In one embodiment, the invention relates to methods for treating a disease, disorder, condition or symptom, comprising administering a liquid form controlled release drug composition to a patient in need thereof, the drug composition comprising:

(a) a dispersed phase comprising an ion-exchange matrix drug complex comprising a pharmaceutically acceptable ion-exchange matrix and a water-soluble electrolytic drug adsorbed onto the ion-exchange matrix, wherein the surface charge of the ion-exchange matrix is opposite that of the electrolytic drug; and

(b) a dispersion medium comprising a polyelectrolyte having the same charge as the electrolytic drug.

The invention relates to methods for treating a disease, condition or symptom, comprising administering a liquid form controlled release drug composition to a patient in need thereof, the drug composition comprising:

(a) a dispersed phase comprising a bead having an ion-exchange matrix drug complex comprising a pharmaceutically acceptable in-exchange matrix and a electrolytic drug, wherein the surface charge of the ion-exchange matrix is opposite that of the electrolytic drug, and a soluble non electrolytic component;

(b) a diffusion-controlling membrane; and

(c) a dispersion medium comprising a highly hydrated excipient.

In one specific embodiment, the ion-exchange matrix drug complex comprises alginic acid, albuterol and lactose; the diffusion-controlling membrane is EUDRAGIT®; and dispersion medium is Syrup NF.

Relative to solid oral dosage forms; liquid formulations have the distinct advantages of dosage flexibility and ease of swallowing. In addition, it is possible to administer, in a single volume of liquid, a relatively large quantity of dispersed solid, which would normally require several tablets or capsules.

The dosage forms of the present invention are useful for treating patients who require chronic administration (e.g., patients with dysphagia or cancer patients receiving morphine).

The dosage forms of the present invention are particularly useful for treating chronic diseases, i.e., diseases that are long-lasting or recurrent. Chronic diseases include but are not limited to cardiovascular diseases (e.g., heart failure, ischemic cardiopathy, cerebrovascular disease, hypertension), autoimmune diseases, cancer, respiratory diseases (e.g., chronic obstructive pulmonary disease, asthma), osteoarticular diseases (e.g., rheumatoid arthritis, osteoarthritis), chronic fatigue syndrome, chronic renal failure, diabetes mellitus, chronic hepatitis, osteoporosis, hypercholesterolemia, chronic pain, and depression.

The dosage forms of the invention are also particularly useful for treating any disease, condition or symptom that requires administration of several active ingredients in the course of treatment, including but not limited to common cold, allergy, cardiovascular disease, hypertension, infectious diseases, respiratory disease, depression, chronic pain, and diabetes.

The dosage forms of the present invention are also particularly useful for treating any disease, condition or symptom that requires administration of one or more drugs that have the potential for abuse and diversion for illegal drug use. Drugs that have the potential for abuse and diversion for illegal drug use include but not limited to opioids (e.g., hydrocodone), pseudoephedrine, ephedrine, barbiturates, benzidiazepines, and amphetamines. Thus, the formulations of the present invention are particularly useful for treating diseases, conditions or symptoms that require administration of such drugs, including but not limited to pain (e.g., chronic pain), cold symptoms (e.g., cough and nasal obstruction or congestion), anxiety, insomnia, epilepsy, depression, narcolepsy, and attention deficit hyperactivity disorder.

The advantages of the dosage forms of the present invention make them particularly useful for treating pediatric and geriatric patient populations. Among other advantages, the compositions of the present invention allow for easy administration because the liquid compositions of the invention are easier to swallow. In certain embodiments, the formulations of the present invention include additional ingredients that improve the taste and flavor of the formulations. In addition, the compositions of the present invention allow for improved patient compliance with drug regimen due to the need for fewer number of doses and/or fewer number of different dosage forms per day. Using the compositions of the invention, patients will need to take medicine less often, and run a smaller risk of missing necessary doses to maintain relief over the course of the day. The above-described advantages of the compositions of the present invention benefit all patient populations, but especially the young (e.g., children) and the elderly. In one embodiment, the patient is less than 18 years of age, less than 12 years of age, less than 10 years of age, less than 5 years of age, less than 2 years of age, or less than 1 year if age. In another embodiment, the patient is at least 40 years of age, 50 years of age, 60 years of age, 65 years of age, or 70 years of age.

In one embodiment, the dosage of the controlled release drug composition used is the dosage sufficient to achieve a therapeutic effect in a patient, for example, in a human patient, wherein the term “therapeutic effect” means any effect against a disease, including but not limited to symptomatic relief, and/or other biological effects resulting in an improvement in subjective well-being. In one such embodiment, the dosage of the controlled release drug composition used is the dosage sufficient to achieve a therapeutic effect in a patient for a time period of at least 8 hours after administering a single dose of a single drug composition to the patient. In another such embodiment, the single drug composition comprises more than one drug, e.g., at least two drug, at least three drugs or at least four drugs, and administration of a single dose of such drug composition achieves sufficient AUCinfinity of all drugs to observe a therapeutic effect in a patient over a period of at least 8 hours after the single dose. In one specific embodiment, the term “therapeutic effect” means any effect against a cold, flu or an allergy, including but not limited to symptomatic relief, such as reducing severity and/or frequency of coughing, symptoms of coughing, nasal discharge, congestion or sneezing, and/or other biological effects resulting in an improvement in subjective well-being.

In one embodiment, the controlled release drug composition is formulated to be dosed once every 6 hours, or once every 8 hours, or once every 12 hours or once every 24 hours, or up to every 48 hours.

In certain embodiments, the liquid form controlled release drug composition is shaken prior to administration to a patient in need thereof.

In another embodiment, the liquid form controlled release drug composition is further diluted by addition of deionized water, a pharmaceutically acceptable liquid that does not interfere with the intended operation of the drug composition, or any combination thereof.

In another embodiment, the invention relates to a kit comprising the solid phase of the liquid form controlled release drug composition comprising an ion-exchange matrix drug complex as described in any above-described embodiments, optionally having a diffusion-controlling membrane; and a dispersion medium, optionally further comprising polyelectrolyte or a highly hydrated excipient. In one embodiment, a kit comprises a first container comprising the solid phase of the liquid form controlled release drug composition, and a second container comprising a dispersion medium, wherein the contents of the first container are mixed with the contents of the second container immediately prior to administration, or stored for 1 month, 3 months, 6 months, or 1 year, 2 years, 3 years, 4 years, 5 years or more prior to administration.

In one embodiment, the invention relates to method for treating a condition or symptom, comprising administering a liquid form controlled release drug composition to a patient in need thereof, comprising:

(a) providing a solid phase of the liquid form controlled release drug composition;

(b) dispersing the solid phase into a pharmaceutically acceptable liquid to provide a dispersion comprising a pharmaceutically effective concentration of the solid phase; and

(c) and administering the dispersion to a patient in need thereof.

In another embodiment, the present invention relates to a method of treating coughing, symptoms of coughing, nasal discharge, congestion or sneezing associated with a cold, flu or an allergy for a time period of at least 8 hours, comprising administering to a human subject in need of such a treatment a single dose of the drug composition of described herein effective to treat coughing, symptoms of coughing, nasal discharge, congestion or sneezing associated with a cold or an allergy, for a time period of at least 8 hours.

In yet another embodiment, the present invention relates to a method for treating cough, cold, flu or allergy symptoms in a human subject, comprising the step of administering the oral extended release drug composition described herein to the subject. In one such embodiment, the pharmaceutical composition is administrated as a dual release formulation allowing a once-a-day or twice-a-day dosing in humans.

Without being bound by particular theories, the controlled release composition is administered to a patient, and drug release can be triggered when the suspension is placed in an environment with a relatively high concentration of water and small ions that possess the same charge as the drug. These endogenous ions, which are counterions relative to the surface of the ion-exchange material, diffuse into and swamp the diffuse double layer, reducing its extent or thickness. Endogenous diffusible counterions with positive charge, such as sodium or potassium, or negative charge, such as chloride or phosphate, generally have higher charge density than the drug and are present in greater concentration than the drug. The result is that these ions essentially free the drug from its electrostatic entrapment, allowing it to diffuse out of the aggregated ion-exchange matrix drug complex.

Drug release can also be triggered by the influx of water or gastric fluid when water hydrates the coating or membrane, thereby increasing its porosity, allowing water inside the bead, and facilitating drug dissolution.

The amount of the composition of the invention which will be effective in the treatment, prevention, management or amelioration of one or more symptoms associated with a condition, disease, or disorder can be determined by standard clinical techniques. For example, the dosage of the composition which will be effective in the treatment, prevention, management or amelioration of one or more symptoms can be determined by administering the composition to animal models known to those skilled in the art. In addition, in vitro assays may optionally be employed to help identify optimal dosage ranges. Selection of the specific effective dose can be determined (e.g., via clinical trials) by a skilled artisan based upon the consideration of several factors which will be known to one of ordinary skill in the art. Such factors include the disease to be treated or prevented, the symptoms involved, the patient's body mass, the patient's immune status and other factors known by the skilled artisan to reflect the accuracy of administered pharmaceutical compositions. The precise dose to be employed in the formulation will also depend the seriousness of the symptoms, and should be decided according to the judgment of the practitioner and each patient's circumstances. Effective doses may be extrapolated from dose-response curves derived from in vitro or animal model test systems.

In preferred embodiments, the dosage forms of the invention are capable of controlled release of drug over a span of 8 hours, 12 hours, or 18 hours, or 24 hours, or up to 48 hours. In certain embodiments, patients need only be dosed once daily. In other embodiment, patients need to be dosed twice daily. The invention encompasses dosage forms that are capable of achieving a linear release profile, preferably with a constant rate of release, or preferably having a zero order release profile.

### 5.3.4 Sustained Release Compositions of the Invention Comprising an Antihistamine, Antitussive and Decongestant in Extended Release Formulations

In one embodiment, the present invention relates to a novel formulation comprising, as active pharmaceutical ingredients (APIs), an antihistamine, an antitussive, and a decongestant, where the formulation exhibits extended release (ER) release of all three drugs. For example, the present invention provides a formulation comprising a novel mixture of immediate release (IR) and ER forms of chlorpheniramine, pseudoephedrine and hydrocodone within a single product. Novel formulations of the present invention include those that result in an IR/ER combination products that can be dosed twice daily with the same effectiveness as previously available IR products comprising all three drugs, or a combination of IR and/or ER products comprising only one or two of the drugs.

An antihistamine inhibits the release or action of histamine in the body, for example by acting as an antihistamine antagonist or inverse agonist at a relevant cell receptor, such as the H₁ receptor. Histamine causes congestion, sneezing, runny and stuffy nose, itching and watery eyes associated with allergies, colds and the flu (influenza). Antihistamines can prevent histamines from attaching to cells and causing such symptoms. Examples of antihistamines include chlorpheniramine, brompheniramine, dimenhydrinate, diphenhydramine, loratadine, meclizine, promethazine and quetiapine. In one embodiment of the present invention, the antihistamine is chlorpheniramine. The term “chlorpheniramine” encompasses any form of the drug, and in one specific embodiment, chlorpheniramine is chlorpheniramine maleate (CPM), also known by the chemical name 2-pyridinepropanamine, -(4-chlorophenyl)-N,N-dimethyl-, (Z)-2-butenedioate (1:1).

Decongestants also can help relieve stuffy nose and congestion caused by a cold or the flu, sinusitis or allergies. Congestion in the nose, sinuses, and chest is due to swollen, expanded, or dilated blood vessels in the membranes of the nose and air passages. These membranes have an abundant supply of blood vessels with a great capacity for expansion (swelling and congestion). Histamine stimulates these blood vessels to expand. Decongestants, by contrast, cause constriction or tightening of the blood vessels in those membranes, which forces much of the blood out of the membranes so that they shrink, and the air passages open up again. Generally, decongestants are chemically related to adrenalin, the natural decongestant, which is also a type of stimulant. The most common oral decongestants are pseudoephedrine and phenylephrine. In one embodiment of the present invention, the decongestant is pseudoephedrine (PSE). The term “pseudoephedrine” encompasses any form of the drug, and in one specific embodiment, pseudoephedrine is pseudoephedrine hydrochloride, also known by the chemical name benzenemethanol, -[1-(methylamino)ethyl]-, [S—(R*,R*)]-, hydrochloride.

Cough medicines are generally grouped into two types: antitussives and expectorants. An antitussive is a medicine used to suppress or relieve coughing, and includes non-narcotic and narcotic antitussives. Benzonatate, dextromethorphan, carbetapentane are examples of non-narcotic antitussives. Dextromethorphan (an antitussive) and guaifenesin (an expectorant) are sometimes combined with each other. One example of a narcotic antitussive is hydrocodone (HC), also an analgesic, which is a semi-synthetic opioid derived from two of naturally occurring opiates, codeine and thebaine. The term “hydrocodone” encompasses any form of the drug. In one embodiment of the present invention, the antitussive is hydrocodone bitartrate, also known by the chemical name morphinan-6-one, 4,5-epoxy-3-methoxy-17-methyl-,(5)-, [R—(R*,R*)]-2,3-dihydroxybutane-dioate (1:1), hydrate (2:5).

Combinations of antihistamines with decongestants are currently commercially available, such as Actifedt, Allegra-D®, Chlor-Trimeton D®, Claritin D®, Contact, Co-Pyronil 2®, Deconamine®, Demazin®, Dimetapp®, Drixoral®, Isoclor®, Nolamine®, Novafed A®, Ornade®, Sudafed Plus®, Tavist Triaminic®, and Trinalin®. Antitussives are also available in combination with other drugs, such as pain relievers or antihistamines. Such combination products, known as multisymptom cold medicines, treat many symptoms at once.

As discussed on the FDA's website, however, FDA-approved IR hydrocodone antitussive formulations contain only hydrocodone bitartrate and homatropine methylbromide, such as in Hycodan®, Mycodone®, and Tussigon®. Only two ER antitussive formulations containing hydrocodone and chlorpheniramine are currently approved, Tussionex Pennkinetic® (suspension) and TussiCaps® (capsule). See www.fda.gov/CDER/drug/unapproved_drugs/hydrocodone_qa.htm; accessed Apr. 11, 2008. Notably, cough suppressants that combine hydrocodone and homatropine with other drugs, like an expectorant such as guaifenesin, or a decongestant such as phenylephrine or pseudoephedrine, are currently unapproved in any form. Thus, no FDA-approved drug comprising hydrocodone and a decongestant, such as PSE, is available.

Consequently, as mentioned above, the present invention differs from previously available combination products because the novel formulations described herein comprise three APIs, i.e., an antihistamine, an antitussive, and a decongestant, and exhibit ER release of all three APIs in the body via a single oral product. In one embodiment, a novel formulation is dosed once every 12 hours. Other embodiments include those dosed every 8 hours, 16 hours, 24 hours, etc.

In one embodiment, the product is a liquid dispersion of ER coated pellets in a syrup intended for the treatment of cough, cold, and allergy symptoms. For example, a formulation may contain 10 or 15 mg hydrocodone bitartrate, 120 mg pseudoephedrine hydrochloride and 8 mg chlorpheniramine maleate in combination per adult dosage (5 ml). Salt forms of the drugs may be used, but other forms of the drugs, including the base forms, may also be used. These active ingredients have extensive human experience dosed either individually or in combination as both prescription and over-the-counter (OTC) cough cold medications.

In one embodiment of the present invention, the ER portion of the formulation corresponds to coated beads, particulates or pellets within a liquid suspension, with an IR portion located in the liquid suspension. In one embodiment, formulations of the present invention are prepared using technology described in published patent applications owned by UPM Pharmaceuticals (see U.S. Ser. No. 10/724,276, filed on Nov. 26, 2003, U.S. Ser. No. 11/150,572 filed on Jun. 9, 2005, and U.S. Ser. No. 11/198,937 filed on Aug. 4, 2005), hereby incorporated by reference in their entirety, which relate to the production and use of a certain type of ER beads in suspension.

Alternatively, the ER portion in the present invention may comprise a solid dosage form such as capsule, tablet, or other oral solid, with an IR portion as a secondary layer or medium outside the ER portion. In certain embodiments, oral solid formulations contain no liquid components, i.e., such formulations do not contain a liquid phase or a dispersion medium. In such embodiments, the IR portion of the oral solid formulations does not comprise a liquid phase or a dispersion medium. In such embodiments, the oral solid formulation is not mixed with a liquid phase, e.g., a dispersion medium, prior to administration to a subject. Likewise, in one embodiment, a single combination ER product exhibits a specific IR to ER ratio, where the ER component is in a particulate, pellet, or bead and the IR portion is outside (e.g., suspended in syrup; in powder in a capsule, tablet, etc.). The ratio achieves blood serum levels that are bioequivalent (BE) to reference listed drugs (RLDs) at single-dose and steady-state conditions.

In other embodiments, administration of the present formulations achieves certain specific blood serum ranges (as measured by AUC, Tₘₐₓ, T_{1/2}, etc.) in humans over time, where the levels are safe and effective for ER 12-hour release and BE to immediate release RLDs at both single-dose and steady-state conditions.

In one embodiment, an oral extended release drug composition comprises a first portion and a second portion, wherein the first portion comprises an antihistamine, an antitussive, and optionally a decongestant, as active ingredients in an immediate release form, and wherein the second portion comprises particulates, pellets, or beads wherein each particulates, pellets, or beads comprises the same antihistamine, antitussive and decongestant as active ingredients in an extended release form. In another embodiment, administration of a single dose of this drug composition to a patient provides serum levels of the three active ingredients over a time period of at least 8 hours, such as 8, 12, 18 or 24 hours, that are bioequivalent to serum levels achieved upon administration of an appropriate number of doses over the same time period of FDA-approved IR reference listed drug (RLD) compositions comprising the active ingredients, wherein the appropriate number of doses corresponds to a number of doses recommended in FDA-approved labels for the administration of the FDA-approved IR drug compositions over the same time period.

In certain embodiments, the extended release portion is in the form of a particulate, pellet or bead. The particulate, pellet or bead is of a size which can be administered orally in a liquid or solid dosage form. In one embodiment, the particulate, pellet or bead is of a size and/or density such that it does not settle in suspension. In some embodiments, the diameter of the particulate, pellet or bead ranges from about 0.01 μm to about 2000 μm; in another embodiment, from about 0.1 μm to about 1000 μm; and in another embodiment, from about 1 μm to about 1000 μm. In one embodiment, the diameter of the particulates, pellets or beads are about 600 μm.

In certain embodiments, a suspension formulation comprising an antihistamine, an antitussive and pseudoephedrine, wherein the formulation exhibits IR and ER release of all three drugs, takes advantage of the fact that pseudoephedrine releases out of ER particulates, pellets or beads more quickly than does an antihistamine (e.g., chlorpheniramine) or antitussive (e.g., hydrocodone). For example, such formulations may comprise the antihistamine, antitussive and pseudoephedrine in ER particulates, pellets or beads, while the IR liquid/vehicle portion comprises the antihistamine and antitussive, but not pseudoephedrine. Certain such formulations still exhibit IR and ER release of all three drugs in a manner that is bioequivalent to the release of the drugs upon administration of corresponding RLDs dosed two or more times as directed on FDA approved labeling, for example over 12 hours.

In certain embodiments, inactive ingredients serving as a carrier for the APIs in formulations of the present invention include: ammonio methacrylate copolymer, lactose monohydrate, methylparaben, microcrystalline cellulose, propylparaben, purified water, sodium alginate, sucrose, talc, titanium dioxide, triethylcitrate. Inert components, such as these, may be used to prepare two distinct phases in formulations of the present invention: a dispersion medium, containing immediate release versions of the drugs, dissolved in syrup; and a dispersed phase comprising coated particulates, pellets or beads containing the extended release portions of the drugs.

Bioequivalence (BE) is a pharmacokinetics term used to describe the in vivo biological equivalence of two preparations of a drug. A bioequivalence requirement refers to a requirement imposed by the FDA for in vitro and/or in vivo testing of specified drug products that must be satisfied as a condition of marketing, under 21 C.F.R. §320.1(f). The U.S. Food and Drug Administration (FDA) has defined bioequivalence as “the absence of a significant difference in the rate and extent to which the active ingredient or active moiety in pharmaceutical equivalents or pharmaceutical alternatives becomes available at the site of drug action when administered at the same molar dose under similar conditions in an appropriately designed study.” 21 C.F.R. §320.1(e); see also “Bioavailability and Bioequivalence Studies for Orally Administered Drug Products General Considerations” published by FDA Jul. 2, 2002, at www.fda.gov/OHRMS/DOCKETS/98fr/02d-0258-gdl0001.pdt formally adopted Mar. 19, 2003 (68 Fed. Reg. 13316 (Mar. 19, 2003)).

BE can be measured by comparing the appropriate pharmacokinetic parameters between the two drugs and determining if they fall within an acceptable limit. “Bioequivalent” serum levels of an active ingredient means that the average log transformed values of AUC_{infinity} measured during a single dose study, and Cₘₐₓ, Cₘᵢₙ, and AUC_{infinity} measured during a steady state study for the active ingredient, as measured in the serum of a patient after administration of a first drug product comprising that active ingredient, is within 80 to 125 percent of the Cₘₐₓ, Cₘᵢₙ and AUC_{infinity} for the active ingredient, as measured in the serum after administration of a second drug product comprising the active ingredient, within a 90% confidence interval.

The FDA recommends a logarithmic transformation of the pharmacokinetic parameters before statistical analysis is done to determine BE. The traditional FDA-recommended BE limits are that the log transformed PK parameters must be within 80 to 125 percent of each other, within a 90% confidence limit. In one embodiment herein, the novel formulation is a 12 hour controlled release drug. This product may be compared to two or three doses of the RLDs dosed two to three times over a 12 hour period, once at t=0 and once at t=6 hours, or once at t=0, once at t=4 hours and once at t=8 hours.

A single dose study is a study in which an ER product of interest is given to patients only once, and its corresponding IR reference listed drugs (RLDs) are dosed for an equivalent 12 hour dose as directed by the FDA approved label on the IR RLDs. A steady state study is a study in which the ER product of interest and IR RLDs are given repeatedly over time during the study until a steady-state blood serum level of the APIs are achieved. The phrase “Cₘₐₓ” refers to the highest serum concentration (e.g., ng/ml) observed in a patient after administration after steady state has been reached. The phrase “Cₘᵢₙ” refers to the lowest serum concentration (e.g., ng/ml) observed in a patient after steady state for the drug has been reached. The phrase “AUC_{infinity}” or “AUC” refers to the area (e.g., ng/ml×hr) under a curve that plots the concentration of an active ingredient in serum over time, from time 0 to infinity, after administration of one or more doses of a drug product over a time period (e.g., 8, 12, 24, 48 hrs, etc.). “Cₘᵢₙ,” “Cₘₐₓ” and “AUC_{infinity}” for an active ingredient may be measured by well known methods.

“Reference listed drug” or “RLD” refers to a listed drug identified by FDA as a drug product upon which an applicant may rely in seeking approval of an abbreviated new drug application (ANDA). Thus, single drug containing RLDs are defined by the FDA. For generic drugs or drugs filed under a 505(b)(2) application, if there is more than one supplier for an API, the FDA selects which supplier will provide the product acting as the RLD. For the purpose of the present invention, RLDs include separate IR drug products containing a single active ingredient. RLDs may be dosed in combination, for example, by administering sequentially per their dosing instructions, for purposes of comparing to formulations of the present invention. Likewise, for the purposes of the present invention, the term “reference listed drug” or “RLD” also refers to a cocktail containing two or more single drug RLDs. For example, an RLD may be a cocktail containing an antihistamine RLD, an antitussive RLD, and a decongestant RLD. For chlorpheniramine, the FDA-recognized RLD is Chlor-Trimeton Syrup. For PSE, the FDA-recognized RLD is Sudafed Syrup. For Hydrocodone, the FDA-recognized RLD is Hycodan Syrup. When comparing serum levels obtained upon administration of a formulation of the present invention and evaluating bioequivalence, one may administer to a human a cocktail containing all three single drug RLDs in a way that complies with FDA-approved labeling for all of the RLDs.

In January 2001, FDA released guidelines describing bioequivalence studies generally, how to set up such studies, how to analyze data, etc., in a document entitled “Guidance for Industry: Statistical Approaches to Establishing Bioequivalence” (“Statistical Approaches”). In this document, the FDA defined the standards that it intended to use to determine if a product has achieved the statutory definition of BE. When referring herein to a product of interest being BE to its reference drug, the definition of “average BE” as described in these documents applies.

As described on page 2 of “Statistical Approaches,” the FDA recommends that “a standard in vivo BE study design be based on the administration of either single or multiple doses of the T (test) and R (reference) products to healthy subjects on separate occasions, with random assignment to the two possible sequences of drug product administration . . . [and that] statistical analysis for pharmacokinetic measures, such as area under the curve (AUC) and peak concentration (Cₘₐₓ), be based on the two one-sided tests procedure to determine whether the average values for the pharmacokinetic measures determined after administration of the T and R products were comparable. This approach is termed average bioequivalence and involves the calculation of a 90% confidence interval for the ratio of the averages (population geometric means) of the measures for the T and R products. To establish BE, the calculated confidence interval should fall within a BE limit, usually 80-125% for the ratio of the product averages.” “Statistical Approaches,” Section LLB, page 2 (emphasis in original).

The statistical analysis of BE data is based on a statistical model for the log transform of the bioavailability data (e.g., AUC, Cₘₐₓ, Cₘᵢₙ). The “Statistical Approaches” guidance suggests that BE measures be log transformed (either natural log or base 10). For data analysis, “Statistical Approaches” recommends using parametric (normal theory) methods for the analysis of log-transformed BE measures. “For average BE using the criterion stated in equations 2 or 3 (section [IV.A]), the general approach is to construct a 90% confidence interval for the quantity μ_{T}-μ_{R} and to reach a conclusion of average BE if this confidence interval is contained in the interval [−θ_{A}, θ_{A}] . . . . The 90% confidence interval for the difference in the means of the log-transformed data should be calculated using methods appropriate to the experimental design.” “Statistical Approaches,” Section VI.B, page 10.

Embodiments of the present invention integrate the benefits of three generally recognized as safe and effective (GRASE) APIs, i.e., an antihistamine, an antitussive, and a decongestant, into one ER medicine. Currently, when used together such drugs are dosed 4-6 times daily in their IR forms because they are not available in a single OTC or prescription-controlled extended release triple-acting combination product. Thus, IR/ER formulations comprising an antihistamine, an antitussive, and a decongestant, where the formulation exhibits ER release of all three drugs upon administration of the single product, provides advantages over currently available products that either do not contain all three active ingredients and/or are merely IR products. For example, upon using the formulations of the current invention, patients will require lower volumes of medicine, e.g., 5 ml as compared to 50 to 55 ml per day of IR products, to achieve relieve from symptoms of a cold, flu or allergy. Formulations of the present invention may also be sold in convenient unit-of-dose 4 ounce containers. Patients will also need to take medicine less often, and run a smaller risk of missing necessary doses to maintain relief over the course of a day. Given that patient compliance is an ever-present and well-recognized problem, a formulation that provides bioequivalent doses of all three drugs in a single dose (e.g. 12 hour dose) offers significant advantages over presently available formulations.

In one embodiment, the present invention provides a formulation comprising a novel mixture of IR and ER forms of chlorpheniramine, pseudoephedrine and hydrocodone within a single product, where the single product is administered to a patient less often, while achieving bioequivalence, in patients administered immediate release product(s) containing these drugs.

The present invention also relates to drug combination formulations and methods of manufacturing such formulations, such as stable oral extended release drugs in a liquid suspension or solid capsules or tablets, that comprise particulates, pellets, or beads having two or more active ingredients contained within each single particulate, pellet, or bead. This approach has multiple advantages not only over IR formulations, but also over ER formulations.

In other embodiments, the invention provides a novel oral liquid suspension formulation comprising an extended-release component comprising pellets, beads or particles containing one or more drugs, where the pellets, beads or particles are suspended in a syrup. The syrup may also contain one or more drugs. The present specification provides an example of such an oral liquid suspension. The oral liquid suspension formulation achieves superior properties over the prior art. For example, because the ER component of the example formulation comprises beads comprising three drugs associates with an ion-exchange matrix, and those beads are suspended in a syrup, the formulation provides greatly increased product stability over other liquid formulations. Typically, drug degradation occurs in an aqueous environment. While degradation can be minimized by the use of solid dosage forms, this prevents the ease of dosing and dosage flexibility found in liquid formulations. In at least one embodiment, the present inventive dosage form minimizes exposure to water by a two step approach: (1) the beads exhibit ER properties, rather than being readily soluble in a liquid phase used to suspend the beads; and (2) the liquid phase is a syrup, where the presence of sugars lower the water activity of the liquid phase. The syrup, by decreasing water activity and increasing osmotic pressure, also serves to: (a) minimize leaching of drugs from the ER beads into the syrup; and (b) prevents degradation of the beads. Conversely, when consumed by a patient, the beads are then able to release the drugs, for example by the beads swelling and degrading in the intestines, allowing for drug release from the ER.

One advantage is that the present formulation achieves bioequivalence for two or more drugs at the same time. Comparative products may not have the same release profile and require different doses or frequency of dosing for each drug. In addition, if different ER technologies are used (e.g. different resins), then the release profile of each drug may be affected by differences in a patient's diet or physiology, such that a given patient may receive too much of one drug, but too little of another. By placing three drugs, for example, in a single bead with a single release technology, the drug release profile will be more consistent across the patient population.

If each of the three drugs are placed in separate beads, which are then mixed, it is possible that the mixture will not be perfectly homogenous, especially in small amounts. Such non-homogeneity will result in incorrect relative doses for the drugs: too high for some, too low for others. Non-homogeneity can occur either due to random fluctuations, but may also be due to different physical properties of the three classes of beads in a three bead class mixture. For example, pseudoephedrine constitutes a larger proportion (by weight) of the drugs. If all three drugs are packaged in individual beads of equivalent size and amount, the greater amount of pseudoephedrine relative to excipient will result in a different density of the pseudoephedrine bead over a hydrocodone or chlorpheniramine bead. Such a density difference would result in a loss of homogeneity. Even if beads are calibrated to be of equivalent density at a given temperature and pressure, this may not hold true under different conditions.

Density difference can also be used to deliberately separate beads according to drug class, and thereby concentrate (say) pseudoephedrine, for diversion into illegal drug use. On the other hand, if all drugs are in a single type of bead, it is not possible to partially isolate one drug from others on the basis of differences in the physical properties of the beads. Moreover, the production of such products will significantly deter or prevent drug abuse or diversion of any one active ingredient present in the particulate, pellet, or bead. By combining or infusing two or more drugs within a single particulate, pellet, or bead, individuals cannot easily extract or separate out individual active ingredients from the products for abuse. In addition, by combining or infusing two or more active ingredients into single particulate, pellet, or bead, one reduces the surface area of particulates, pellets, or beads present in an overall drug combination product, thereby providing increased stability of the product and active ingredients.

In one embodiment, a drug combination product will have a decreased potential for separation or isolation of a single active ingredient by comprising particulates, pellets, or beads having two or more active ingredients per particulate, pellet, or bead. Likewise, the present invention provides methods for manufacturing cold and allergy combination drug formulations that have less abuse potential with regard to any single ingredient included in the formulation. For example, each bead within a product may comprise a pharmaceutically acceptable ion-exchange matrix and two or more pharmaceutically acceptable active ingredient drugs associated with the ion-exchange matrix, as such as those prepared using technology described in published patent applications owned by UPM Pharmaceuticals (see, e.g., U.S. Ser. No. 10/724,276, U.S. Ser. No. 11/150,572 and U.S. Ser. No. 11/198,937, hereby incorporated by reference in its entirety), relating to the production and use of a certain type of ER beads in suspension. From such a product, one cannot easily extract, separate or isolate any single active ingredient drug from the drug combination product in a makeshift laboratory.

Thus, formulations of the current invention differ from others currently available in the cough/cold market. For example, Tussionex® Extended-Release Suspension is a cough-suppressant/antihistamine combination, comprising hydrocodone and chlorpheniramine, used to relieve coughs and the upper respiratory symptoms of colds and allergies. This liquid suspension product contains drug-ion exchange resin beads, where any individual beads in the suspension is impregnated with either hydrocodone or chlorpheniramine, but not both drugs in any single bead.

Formulations of the present invention will also differ from those currently available in that one will not be able to readily rely on common household products and easily assessable equipment, or “recipes” for making or isolating a drug of abuse via the Internet, with formulations of the present invention. Rather, separation of a potential drug of abuse of interest from formulations of the present invention will require high quality state of the art equipment and scientific training and technology, e.g., complex chromatography, normally only available in academic or industrial laboratories.

In another embodiment of the present invention, a drug combination product has a decreased potential for abuse and/or diversion by comprising ion-exchange matrix drug particulates, pellets, or beads. Each bead may comprise a pharmaceutically acceptable ion-exchange matrix and two or more pharmaceutically acceptable active ingredient drugs associated with the ion-exchange matrix.

The invention also includes methods for preventing or reducing abuse of at least one active ingredient, comprising preparing a drug combination product, wherein the product comprises particulates, pellets, or beads, wherein each particulate, pellet, or bead comprises a pharmaceutically acceptable ion-exchange matrix and two or more pharmaceutically acceptable active ingredient drugs associated with the ion-exchange matrix.

In another embodiment, methods for preventing or reducing the ability to extract, isolate or separate out a single active ingredient comprise preparing a drug combination product, wherein that product comprises particulates, pellets, or beads, wherein each particulate, pellet, or bead comprises a pharmaceutically acceptable ion-exchange matrix and two or more pharmaceutically acceptable active ingredient drugs associated with the ion-exchange matrix. The fact that the matrix particulate, pellet, or bead comprises two or more active ingredients makes extraction or separation of any single drug exceedingly difficult for an untrained individual in a makeshift laboratory lacking industrial grade or other high quality equipment, such as chromatography equipment.

In another embodiment, drug combination products of the present invention allow for appropriate and precise dosing by a patient of, for example, three different active ingredients. When patients self-medicate with multiple compositions (e.g., three different products, where each contains a single IR active ingredient), appropriate and precise dosing is often difficult, especially with regard to avoiding over- or under-dosing of one or more drugs, and/or maximizing therapeutic benefit of all drugs while minimizing side effects. Combination products of the present invention avoid such difficulties because all relevant drugs are supplied and administered in single dose forms, such as in a 12-hour ER form.

In one embodiment, the drug combination product is a liquid suspension composition comprising a dispersed phase comprising coated ion-exchange matrix drug particulates, pellets, or beads containing extended released drugs, optionally comprising a dispersion medium containing immediate release drugs, dissolved in syrup.

In one specific embodiment, the present invention relates to an oral extended release drug composition comprising a first portion and a second portion, wherein

the first portion comprises an antihistamine, an antitussive, and optionally a decongestant as active ingredients in an immediate release form,

the second portion comprises a particulate, pellet, or bead that comprises the antihistamine, the antitussive, and the decongestant as three active ingredients in an extended release form,

administration of a single dose of the oral drug composition to a patient provides serum levels of the three active ingredients over a time period of at least 8 hours that are bioequivalent to serum levels achieved upon administration of an appropriate number of doses, over the same time period, of FDA-approved immediate release reference listed drug (IR RLD) compositions comprised of the active ingredients, and

the appropriate number of doses corresponds to a number of doses recommended in one or more FDA-approved labels for administration of the IR RLD compositions over the same time period.

In one embodiment of the above-described composition, the antitussive is a narcotic antitussive. In another embodiment of the above-described composition, the first portion does not comprise the decongestant, and comprises only an antihistamine and an antitussive. In a specific embodiment the antihistamine is chlorpheniramine. In another specific embodiment, the antitussive is hydrocodone. In yet another specific embodiment, the decongestant is pseudoephedrine. In certain embodiments, the above-described drug composition is in an oral liquid suspension form. In one embodiment of the above-described composition, the particulate, pellet or bead further comprises a coating.

In a certain specific embodiment of the above-described composition, the antihistamine, the antitussive and the decongestant associate in a single particulate, pellet or bead. In such embodiment, the particulate, pellet or bead further comprises a pharmaceutically acceptable ion-exchange matrix, wherein the antihistamine, the antitussive and the decongestant associate with the ion-exchange matrix.

Some embodiments of the above-described composition further comprise an excipient selected from the group consisting of sweetening agents, flavoring agents, coloring agents, and any combination thereof. Other embodiments further comprise an additive selected from the group consisting of stabilizing agents, dispersion agents, and any combination thereof.

In a certain embodiment, the present invention envisages an oral extended release drug composition comprising a first portion and a second portion, wherein

the first portion comprises an antihistamine, an antitussive, and optionally a decongestant, as active ingredients in an immediate release form,

the second portion is a particulate, pellet or bead that comprises the antihistamine, the antitussive, and the decongestant as active ingredients in an extended release form,

administration of a sufficient number of doses of the drug composition to a patient to achieve steady-state serum levels of the three active ingredients over a time period of greater than 24 hours yields serum levels of the active ingredients that are bioequivalent to serum levels achieved upon administration of an appropriate number of doses, over the same time period, of one or more FDA-approved immediate release drug compositions comprised of the active ingredients, and

the appropriate number of doses corresponds to a number of doses recommended in one or more FDA-approved labels for the administration of the one or more FDA-approved immediate release drug compositions over the same time period.

In another embodiment, the present invention relates to a method for treating cough, cold, flu or allergy symptoms in a human subject, comprising the step of administering the oral extended release drug composition described herein to the subject. In one such embodiment, the pharmaceutical composition is administered as a dual release formulation allowing a one-a-day or twice-a-day dosing in humans.

In yet another embodiment, the present invention relates to a method of treating coughing, symptoms of coughing, nasal discharge, congestion or sneezing associated with a cold, flu or an allergy for a time period of at least 8 hours, comprising administering to a human subject in need of such a treatment a single dose of the drug composition described herein effective to treat coughing, symptoms of coughing, nasal discharge, congestion or sneezing associated with a cold or an allergy, for a time period of at least 8 hours.

In yet another embodiment, the present invention relates to a method for making the liquid oral extended release drug composition described herein, comprising

preparing the immediate release portion, wherein the immediate release portion comprises an antihistamine, an antitussive, and optionally a decongestant as active ingredients;

preparing the extended release portion, which comprises preparing particulates, pellets or beads comprising an antihistamine, an antitussive, and a decongestant as active ingredients;

coating the particulates, pellets or beads with a membrane coating; and

combining the extended release portion with the immediate release portion.

In certain embodiments, the above-described method further comprises preparing particulates, pellets or beads, wherein two or more active ingredients associate in a single particulate, pellet or bead. In one specific embodiment, such method further comprises the step of associating the two or more active ingredients with a pharmaceutically acceptable ion-exchange matrix. In a specific embodiment the antihistamine is chlorpheniramine. In another specific embodiment, the antitussive is hydrocodone. In yet another specific embodiment, the decongestant is pseudoephedrine.

In another embodiment, the present invention relates to a method for achieving in a mammal serum levels of an antihistamine, an antitussive and a decongestant over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:

administering to the mammal an oral extended release drug composition comprising a first portion and a second portion, wherein the first portion comprises the antihistamine, the antitussive and optionally the decongestant as active ingredients in an immediate release form, and wherein the second portion comprises a particulate, pellet, or bead that comprises the antihistamine, antitussive and the decongestant as active ingredients in an extended release form, and

achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

In yet another embodiment, the present invention relates to a method for achieving in a mammal steady-state serum levels of an antihistamine, an antitussive and a decongestant upon administration of an oral extended release (ER) drug composition, wherein the serum levels are bioequivalent to serum levels achieved upon administration of one or more immediate release (IR) compositions comprising active ingredients and inactive ingredients, wherein said active ingredients consist of chlorpheniramine, hydrocodone and pseudoephedrine to the same mammal, wherein the method comprises:

administering to the mammal an oral ER drug composition comprising a first portion and a second portion, wherein the first portion comprises the antihistamine, the antitussive and the decongestant as active ingredients in an immediate release form, and wherein the second portion comprises a particulate, pellet or bead that comprises the antihistamine, the antitussive and the decongestant as active ingredients in an extended release form, and

wherein administration of a sufficient number of doses of the oral ER drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,

wherein the appropriate number of doses of the one or more FDA-approved IR drug compositions corresponds to a number of doses recommended in one or more FDA-approved labels for the administration of the one or more FDA-approved IR drug compositions over the same time period, and

wherein the appropriate number of doses of the one or more FDA-approved IR drug compositions is greater than the sufficient number of doses of the oral ER drug composition.

In one embodiment, the present invention relates to an oral extended release drug composition comprising a first portion and a second portion, wherein

the first portion comprises chlorpheniramine, hydrocodone, and optionally pseudoephedrine as active ingredients in an immediate release form,

the second portion comprises a particulate, pellet, or bead that comprises chlorpheniramine, hydrocodone and pseudoephedrine as three active ingredients in an extended release form,

administration of a single dose of the oral drug composition to a patient provides serum levels of the three active ingredients over a time period of at least 8 hours that are bioequivalent to serum levels achieved upon administration of an appropriate number of doses over the same time period of FDA-approved immediate release reference listed drug (IR RLD) compositions comprising the active ingredients, and

the appropriate number of doses corresponds to a number of doses recommended in one or more FDA-approved labels for the administration of the IR RLD compositions over the same time period.

In some embodiments of the above-described drug composition, the time period of at least 8 hours is 12 hours. In other embodiments, the time period of at least 8 hours is 24 hours.

In some embodiments of the above-described drug composition, the first portion does not comprises pseudoephedrine.

In one embodiment of the above-described drug composition, the drug composition is in an oral liquid suspension form. In another embodiment, the drug composition is in an oral solid form. In yet another embodiment, the drug composition is in an oral capsule form.

In some embodiments of the above-described drug composition, the particulate, pellet or bead further comprises a coating. In some such embodiments, the particulate, pellet or bead further comprises a pharmaceutically acceptable ion-exchange matrix, wherein the chlorpheniramine, hydrocodone and pseudoephedrine associate with the ion-exchange matrix.

In certain embodiments, the above-described drug composition further comprises an excipient selected from the group consisting of sweetening agents, flavoring agents, coloring agents, and any combination thereof. In certain embodiments, the drug composition further comprises an additive selected from the group consisting of stabilizing agents, dispersion agents, and any combination thereof.

In some embodiments of the above-described drug composition, the time period of at least 8 hours is 12 hours, and the drug composition comprises 8 to 12 mg chlorpheniramine maleate, 10 to 15 mg hydrocodone bitartrate and at least 120 mg pseudoephedrine per 5 ml single dose. In other embodiments, the time period of at least 8 hours is 24 hours, and the drug composition comprises 16 to 24 mg chlorpheniramine maleate, 20 to 30 mg hydrocodone bitartrate and at least 240 mg pseudoephedrine hydrochloride per 5 ml single dose.

In another embodiment, the present invention envisages a method for treating coughing, symptoms of coughing, nasal discharge, congestion or sneezing associated with a cold, flu or an allergy for a time period of at least 8 hours, comprising administering to a human subject in need of such a treatment a single dose of the drug composition described herein effective to treat coughing, symptoms of coughing, nasal discharge, congestion or sneezing associated with a cold or an allergy, for the time period of at least 8 hours.

In certain embodiments of the above-described method, the time period of at least 8 hours is 12 hours. In another embodiment, the time period of at least 8 hours is 24 hours.

In a certain embodiment, the present invention encompasses an oral pharmaceutical formulation comprising chlorpheniramine, hydrocodone and pseudoephedrine as active ingredients, wherein the formulation exhibits immediate release (IR) and extended release (ER) of the active ingredients, wherein

the formulation comprises an immediate release portion and an extended release portion, and

administration of a single dose of the oral formulation to a patient provides serum levels of chlorpheniramine, hydrocodone and pseudoephedrine over a time period of at least 8 hours that are bioequivalent to serum levels achieved upon administration of two or more doses, over the same time period, of one or more IR compositions comprising chlorpheniramine, hydrocodone and/or pseudoephedrine.

In one embodiment of the above-described formulation, the time period of at least 8 hours is 12 hours. In another embodiment, the time period of at least 8 hours is 24 hours.

In some embodiments, the above-described formulation is in an oral liquid suspension form.

In certain embodiments, the present invention relates to a method of making the oral pharmaceutical formulation described above, comprising preparing the immediate release portion, wherein the immediate release portion comprises chlorpheniramine and hydrocodone, but not pseudoephedrine.

In other embodiments, the present invention contemplates a method of making the oral pharmaceutical formulation described above, comprising preparing the extended release portion, which comprises preparing particulates, pellets or beads, wherein each individual particulate, pellet or bead comprises chlorpheniramine, hydrocodone and pseudoephedrine,

wherein the method further comprises combining the extended release portion with the immediate release portion.

In some embodiments, the above-described method, further comprises coating the particulates, pellets or beads with a membrane coating prior to combining the extended release portion with the immediate release portion.

In certain embodiments, the present invention relates to an oral extended release drug composition comprising a first portion and a second portion, wherein

the first portion comprises chlorpheniramine, hydrocodone, and optionally pseudoephedrine as active ingredients in an immediate release form,

the second portion is a particulate, pellet or bead that comprises chlorpheniramine, hydrocodone and pseudoephedrine as active ingredients in an extended release form,

administration of a sufficient number of doses of the drug composition to a patient to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release drug compositions comprising the active ingredients, and

the appropriate number of doses corresponds to a number of doses recommended in one or more FDA-approved labels for the administration of the one or more FDA-approved immediate release drug compositions over the same time period.

In another embodiment, the present invention relates to an oral extended release drug composition comprising a first portion and a second portion, wherein

the first portion comprises chlorpheniramine, hydrocodone, and optionally pseudoephedrine as active ingredients in an immediate release form,

the second portion comprises a particulate, pellet, or bead that comprises chlorpheniramine, hydrocodone and pseudoephedrine as active ingredients in an extended release form,

administration of a single dose of the drug composition to a patient provides serum levels of the three active ingredients over a time period of at least 8 hours that are bioequivalent to serum levels achieved upon administration of an appropriate number of doses over the same time period of an FDA-approved immediate release reference listed drug (IR RLD) composition comprising all three active ingredients, and

the appropriate number of doses corresponds to a number of doses recommended in an FDA-approved label for the administration of the IR RLD composition over the same time period.

In yet another embodiment, the present invention relates to an oral pharmaceutical composition comprising: (1) an immediate release (IR) portion comprising chlorpheniramine and hydrocodone as active ingredients, and (2) an extended release (ER) portion comprising chlorpheniramine, hydrocodone and pseudoephedrine as active ingredients, wherein

the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

administration of a single dose of the oral composition provides an AUC_{infinity} for hydrocodone in a human subject that is equivalent to an AUC_{infinity} obtained upon administration of two or more doses of an immediate release reference listed drug (IR RLD) having one half or less of the amount of hydrocodone present in the oral composition, and

administration of a single dose of the oral composition provides an AUC_{infinity} for pseudoephedrine in a human subject that is equivalent to an AUC_{infinity} obtained upon administration of two or more doses of an immediate release reference listed drug (IR RLD) having one half or less of the amount of pseudoephedrine present in the oral composition.

In some embodiments of the oral composition described above, administration of a single dose of the oral composition provides an AUC_{infinity} for chlorpheniramine in a human subject that is equivalent to an AUC_{infinity} obtained upon administration of two or more doses of an immediate release reference listed drug (IR RLD) having one half or less of the amount of chlorpheniramine present in the oral composition.

In certain other embodiments, the present invention relates to an oral pharmaceutical composition comprising: (1) an immediate release (IR) portion comprising chlorpheniramine and hydrocodone as active ingredients, and (2) an extended release (ER) portion comprising chlorpheniramine, hydrocodone and pseudoephedrine as active ingredients, wherein

the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

the oral composition demonstrates an AUC_{infinity} for hydrocodone in a human subject that is equivalent to an AUC_{infinity} obtained upon administration of two doses of an immediate release reference listed drug (IR RLD) having one half the amount of hydrocodone as compared to the oral composition, wherein the oral composition is dosed once, and the IR RLD is dosed twice at zero and six hours, over a 12 hour period, and

the oral composition demonstrates an AUC_{infinity} for pseudoephedrine in a human subject equivalent to an AUC_{infinity} obtained upon administration of two doses of an IR RLD having one half the amount of pseudoephedrine as compared to the oral composition, wherein the oral composition is dosed once, and the IR RLD is dosed twice at zero and six hours, over a 12 hour period.

In some specific embodiments, the above-described oral composition demonstrates an AUC_{infinity} for chlorpheniramine in a human subject equivalent to an AUC_{infinity} obtained upon administration of two doses of an IR RLD having one half the amount of chlorpheniramine as compared to the oral composition, wherein the oral composition is dosed once, and the IR RLD is dosed twice at zero and six hours, over a 12 hour period.

In one embodiment the present invention envisages a method for treating cough, cold, flu or allergy symptoms in a human subject, comprising the step of administrating one of the oral extended release drug compositions described herein to the subject.

In certain embodiments of the above-described method, the pharmaceutical composition is administrated as a dual release formulation allowing a one-a-day or twice-a-day dosing in humans.

In some embodiments, the present invention relates to an oral extended-release drug composition comprising an antihistamine, an antitussive and a decongestant as active ingredients, wherein the composition provides sufficient AUC_{infinity} of all three active ingredients to achieve a therapeutic effect for a time period of at least 8 hours after a single dose in a human subject, according to serum analysis.

In one specific embodiment, the present invention relates to an oral extended-release drug composition comprising chlorpheniramine, hydrocodone and pseudoephedrine as active ingredients, wherein the composition provides sufficient AUC_{infinity} of all three active ingredients to achieve a therapeutic effect for a time period of at least 8 hours after a single dose in a human subject, according to serum analysis.

In the context of the embodiments described above, the term “therapeutic effect” means any effect against a cold, flu or an allergy, including but not limited to symptomatic relief, such as reducing severity and/or frequency of coughing, symptoms of coughing, nasal discharge, congestion or sneezing, and/or other biological effects resulting in an improvement in subjective well-being.

In one embodiment of the above-described drug composition, the time period of at least 8 hours is 12 hours. In yet another embodiment of the above-described drug composition, the time period of at least 8 hours is 24 hours.

In one embodiment the present invention encompasses a method for preventing or reducing an ability to extract, isolate or separate out pseudoephedrine or ephedrine present in an oral extended-release drug composition, comprising:

preparing the oral extended release drug composition so that it comprises a first portion and a second portion, wherein

the first portion comprises an antihistamine, an antitussive, or both in immediate release form, and optionally comprises pseudoephedrine or ephedrine, and

the second portion comprises particulates, pellets, or beads, wherein each particulate, pellet, or bead comprises pseudoephedrine or ephedrine, and the antihistamine or antitussive or both, as active ingredients in an extended release form, and

preventing or reducing the ability to extract, isolate or separate out pseudoephedrine or ephedrine present in an oral extended-release drug composition.

In one specific embodiment of the above described method, the antitussive is hydrocodone, and wherein the method further comprises preventing or reducing an ability to extract, isolate or separate out hydrocodone present in the oral extended-release drug composition.

In another embodiment, the above-described method further comprises a step of manufacturing that makes extraction, isolation or separation of the pseudoephedrine or ephedrine from the oral extended-release drug composition more difficult, as compared to an immediate release composition comprising pseudoephedrine or ephedrine.

In certain embodiments the present invention envisages a method of reducing the abuse potential of pseudoephedrine or ephedrine present in an oral extended-release drug composition, comprising:

preparing the oral extended release drug composition so that it comprises a first portion and a second portion, wherein

the first portion comprises an antihistamine, an antitussive or both in immediate release form, and optionally comprises pseudoephedrine or ephedrine, and

the second portion comprises a particulate, pellet, or bead that comprises the antihistamine, the antitussive, and pseudoephedrine or ephedrine, as active ingredients in an extended release form.

In certain other embodiments the present invention envisages a method for reducing the abuse potential of a narcotic antitussive or pseudoephedrine present in an oral extended-release drug composition, comprising preparing the oral extended release drug composition so that it comprises a particulate, pellet, or bead comprising the narcotic antitussive and pseudoephedrine as active ingredients in an extended release form.

In one embodiment the present invention relates to a method for manufacturing a solid oral extended release combination drug formulation for use in the treatment of symptoms of a cold, flu or allergy, wherein the formulation has reduced abuse potential with regard to pseudoephedrine or ephedrine included in the formulation, as compared to an immediate release (IR) formulation comprising pseudoephedrine or ephedrine, wherein the method comprises:

preparing the solid oral extended release drug composition so that it comprises particulates, pellets, or beads, wherein each particulate, pellet, or bead comprises two or more active ingredients in an extended release form, wherein at least one of the active ingredients is pseudoephedrine or ephedrine, and wherein at least one of the active ingredients is not pseudoephedrine or ephedrine.

In another embodiment the present invention relates to a method for preventing or reducing the ability to extract, isolate or separate out pseudoephedrine or ephedrine present in a solid oral extended-release drug composition, comprising:

preparing the solid oral extended release drug composition comprising particulates, pellets, or beads, wherein each particulate, pellet, or bead comprises two or more pharmaceutically acceptable active ingredients in an extended release form, wherein at least one of the active ingredients is pseudoephedrine or ephedrine, and wherein at least one of the active ingredients is not pseudoephedrine or ephedrine.

In yet another embodiment, the present invention contemplates a method for achieving in a mammal serum levels of three active ingredients over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:

(A) administering to the mammal an oral extended release drug composition comprising a first portion and a second portion, wherein the first portion comprises an antihistamine, an antitussive, and optionally a decongestant, as active ingredients in an immediate release form, and wherein the second portion comprises a particulate, pellet, or bead that comprises the antihistamine, the antitussive and the decongestant as three active ingredients in an extended release form, and

(B) achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

In yet another embodiment, the present invention contemplates a method for achieving in a mammal serum levels of chlorpheniramine, hydrocodone and pseudoephedrine over a time period of at least 8 hours that are bioequivalent to serum levels achieved upon administration of two or more doses over the same time period of one or more immediate release (IR) compositions comprising chlorpheniramine, hydrocodone and/or pseudoephedrine to the same mammal, wherein the method comprises:

(A) administering to the mammal a single oral pharmaceutical formulation comprising chlorpheniramine, hydrocodone and pseudoephedrine as active ingredients, wherein the formulation exhibits IR and extended release (ER) of the active ingredients, wherein the formulation comprises an IR portion and an ER portion, and

(B) achieving serum levels of chlorpheniramine, hydrocodone and pseudoephedrine over a time period of at least 8 hours that are bioequivalent to serum levels achieved upon administration of two or more doses over the same time period of one or more IR compositions comprising chlorpheniramine, hydrocodone and/or pseudoephedrine.

In some embodiments of the above-described method, the time period of at least 8 hours is 12 hours. In other embodiments of the above-described method, the time period of at least 8 hours is 24 hours.

In yet another embodiment, the present invention contemplates a method for achieving in a mammal steady-state serum levels of an antihistamine, an antitussive and a decongestant upon administration of an oral extended release (ER) drug composition, wherein the serum levels are bioequivalent to serum levels achieved upon administration of one or more immediate release (IR) compositions comprising the antihistamine, the antitussive and/or the decongestant to the same mammal, wherein the method comprises:

administering to the mammal an oral ER drug composition comprising a first portion and a second portion, wherein the first portion comprises an antihistamine, an antitussive, and optionally a decongestant, as active ingredients in an immediate release form, and wherein the second portion is a particulate, pellet or bead that comprises the antihistamine, the antitussive and the decongestant as active ingredients in an extended release form, and

wherein administration of a sufficient number of doses of the oral ER drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,

wherein the appropriate number of doses of the one or more FDA-approved IR drug compositions corresponds to a number of doses recommended in one or more FDA-approved labels for the administration of the one or more FDA-approved IR drug compositions over the same time period, and

wherein the appropriate number of doses of the one or more FDA-approved IR drug compositions is greater than the sufficient number of doses of the oral ER drug composition.

In certain embodiments the present invention relates to a method for achieving in a mammal serum levels of an antihistamine, an antitussive and a decongestant as active ingredients over a time period of at least 8 hours that are bioequivalent to serum levels achieved upon administration of an appropriate number of doses over the same time period of an FDA-approved immediate release reference listed drug (IR RLD) composition comprising all three active ingredients to the same mammal, wherein the method comprises:

(A) administering to the mammal a single dose of an oral extended release (ER) drug composition comprising a first portion and a second portion,

wherein the first portion comprises the antihistamine, the antitussive, and optionally the decongestant, as active ingredients in an IR form,

wherein the second portion is a particulate, pellet, or bead that comprises the antihistamine, the antitussive and the decongestant as active ingredients in an ER form,

(B) achieving serum levels of all three active ingredients over a time period of at least 8 hours that are bioequivalent to serum levels achieved upon administration of an appropriate number of doses over the same time period of an FDA-approved IR RLD composition comprising all three active ingredients, and

wherein the appropriate number of doses corresponds to a number of doses recommended in an FDA-approved label for the administration of the IR RLD composition over the same time period.

In certain other embodiments the present invention relates to a method for achieving AUC_{infinity} values for hydrocodone and pseudoephedrine in a mammalian subject, wherein the method comprises:

(A) administering at the beginning of a time period of at least eight hours a single dose of an extended release (ER) oral pharmaceutical composition comprising: (1) an immediate release (IR) portion comprising chlorpheniramine and hydrocodone as active ingredients, and (2) an ER portion comprising chlorpheniramine, hydrocodone and pseudoephedrine as active ingredients,

wherein the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

(B) achieving an AUC_{infinity} for hydrocodone in a mammalian subject that is equivalent to an AUC_{infinity} obtained upon administrating over the same time period two or more doses of an immediate release reference listed drug (IR RLD) having one half or less of the amount of hydrocodone present in the oral composition, and

C) achieving an AUC_{infinity} for pseudoephedrine in a mammalian subject that is equivalent to an AUC_{infinity} obtained upon administrating over the same time period two or more doses of an immediate release reference listed drug (IR RLD) having one half or less of the amount of pseudoephedrine present in the oral composition.

In one embodiment, the above-described method further comprises: D) achieving an AUC_{infinity} for chlorpheniramine in a mammalian subject that is equivalent to an AUC_{infinity} obtained upon administrating over the same time period two or more doses of an immediate release reference listed drug (IR RLD) having one half or less of the amount of pseudoephedrine present in the oral composition.

In another embodiment the present invention relates to a method for achieving AUC_{infinity} values for hydrocodone and pseudoephedrine in a mammalian subject, wherein the method comprises:

(A) administering an extended release (ER) oral pharmaceutical composition comprising: (1) an immediate release (IR) portion comprising chlorpheniramine and hydrocodone as active ingredients, and (2) an ER portion comprising chlorpheniramine, hydrocodone and pseudoephedrine as active ingredients,

wherein the weight ratio of chlorpheniramine in the IR portion to the ER portion of the oral composition is about 25:75, and the weight ratio of hydrocodone in the IR portion to the ER portion is about 25:75, and the weight ratio of pseudoephedrine in the IR portion to the ER portion is about 0:100,

(B) achieving an AUC_{infinity} for hydrocodone in a mammalian subject that is equivalent to an AUC_{infinity} obtained upon the administration of two doses of an immediate release reference listed drug (IR RLD) having one half the amount of hydrocodone as compared to the oral composition, wherein the oral composition is dosed once, and the IR RLD is dosed twice at zero and six hours, over a 12 hour period, and

C) achieving an AUC_{infinity} for pseudoephedrine in a mammalian subject equivalent to an AUC_{infinity} obtained upon the administration of two doses of an IR RLD having one half the amount of pseudoephedrine as compared to the oral composition, wherein the oral composition is dosed once, and the IR RLD is dosed twice at zero and six hours, over a 12 hour period.

In one embodiment, the above-described method further comprises: D) achieving an AUC_{infinity} for chlorpheniramine in a mammalian subject equivalent to an AUC_{infinity} obtained upon the administration of two doses of an IR RLD having one half the amount of chlorpheniramine as compared to the oral composition, wherein the oral composition is dosed once, and the IR RLD is dosed twice at zero and six hours, over a 12 hour period.

In yet other embodiments, the present invention envisages a method for providing sufficient AUC_{infinity} of an antihistamine, an antitussive and a decongestant to achieve a therapeutic effect in a human subject for a time period of at least 8 hours after administering a single dose of a single drug composition to the human subject, wherein the method comprises: (A) administering to the human subject a single dose of a single oral extended-release drug composition comprising chlorpheniramine, hydrocodone and pseudoephedrine as active ingredients, and (B) achieving sufficient AUC_{infinity} of all three active ingredients to observed a therapeutic effect in the human subject over a period of at least 8 hours after the single dose, according to serum analysis.

In a specific embodiment, the present invention envisages a method for providing sufficient AUC_{infinity} of chlorpheniramine, hydrocodone and pseudoephedrine to achieve a therapeutic effect in a human subject for a time period of at least 8 hours after administering a single dose of a single drug composition to the human subject, wherein the method comprises: (A) administering to the human subject a single dose of a single oral extended-release drug composition comprising chlorpheniramine, hydrocodone and pseudoephedrine as active ingredients, and (B) achieving sufficient AUC_{infinity} of all three active ingredients to observed a therapeutic effect in the human subject over a period of at least 8 hours after the single dose, according to serum analysis.

In one embodiment of the above-described method, the time period of at least 8 hours is 12 hours. In another embodiment, the time period of at least 8 hours is 24 hours.

In some embodiments, the present invention relates to a solid oral extended release drug composition comprising a first portion and a second portion,

wherein the first portion comprises an antihistamine, an antitussive, and optionally a decongestant, as active ingredients in an immediate release form,

wherein the second portion comprises a particulate, pellet, or bead that comprises the antihistamine, the antitussive and the decongestant as three active ingredients in an extended release form,

wherein administration of a single dose of the oral drug composition to a patient provides serum levels of the three active ingredients over a time period of at least 8 hours that are bioequivalent to serum levels achieved upon administration of an appropriate number of doses over the same time period of FDA-approved immediate release reference listed drug (IR RLD) compositions comprising the active ingredients, and

wherein the appropriate number of doses corresponds to a number of doses recommended in one or more FDA-approved labels for the administration of the IR RLD compositions over the same time period.

In other embodiments, the present invention encompasses a method for manufacturing a solid oral extended release combination drug formulation for use in the treatment of symptoms of a cold, flu or allergy, wherein the formulation has reduced abuse potential with regard to any single active ingredient included in the formulation, as compared to an immediate release (IR) formulation comprising the active ingredient, wherein the method comprises:

preparing the solid oral extended release drug composition so that it comprises particulates, pellets, or beads, wherein each particulate, pellet, or bead comprises two or more active ingredients in an extended release form.

In yet another embodiment, the present invention relates to a method for preventing or reducing the ability to extract, isolate or separate out a single active ingredient present in a solid oral extended-release drug composition, comprising:

preparing the solid oral extended release drug composition comprising particulates, pellets, or beads, wherein each particulate, pellet, or bead comprises two or more pharmaceutically acceptable active ingredients in an extended release form.

In some embodiments, the present invention envisages a method for preventing or reducing the ability to extract, isolate or separate out pseudoephedrine or ephedrine present in an oral extended-release drug composition, wherein the method comprises preparing the oral extended release drug composition so that it comprises particulates, pellets, or beads, wherein each particulate, pellet, or bead comprises the pseudoephedrine or ephedrine, and an antihistamine or an antitussive or both, as active ingredients in an extended release form.

In some other embodiments the present invention relates to a method of reducing the abuse potential of pseudoephedrine or ephedrine present in an oral extended-release drug composition, wherein the method comprises preparing the oral extended release drug composition so that it comprises particulates, pellets, or beads, wherein each particulate, pellet, or bead comprises an antihistamine, an antitussive, and the pseudoephedrine or ephedrine, as active ingredients in an extended release form.

In another specific embodiment, an oral extended release drug composition comprises active ingredients consisting of an antihistamine, an antitussive and a decongestant. In one such embodiment, the immediate release portion of the drug composition comprises the active ingredients consisting of an antihistamine, an antitussive and optionally a decongestant. In another such embodiment, the extended release portion of the drug composition comprises the active ingredients consisting of an antihistamine, an antitussive and a decongestant.

In yet another specific embodiment, an oral extended release drug composition comprises active ingredients consisting of chlorpheniramine, hydrocodone and pseudoephedrine. In one such embodiment, the immediate release portion of the drug composition comprises the active ingredients consisting of chlorpheniramine, hydrocodone and optionally pseudoephedrine. In another such embodiment, the extended release portion of the drug composition comprises the active ingredients consisting of chlorpheniramine, hydrocodone and pseudoephedrine.

The following examples are set forth to assist in understanding the invention and should not be construed as specifically limiting the invention described and claimed herein. Such variations of the invention, including the substitution of all equivalents now known or later developed, which would be within the purview of those skilled in the art, and changes in formulations or minor changes in experimental design, fall within the scope of the present invention.

### 6. EXAMPLES

### 6.1 Example 1

Example 1 describes a method for making a calcium alginate propranolol hydrochloride ion-exchange matrix drug complex.

A 2% dispersion of sodium alginate in deionized water was prepared and 500 mL was added via a fluid-metering pump at a rate of approximately 1 mL/min pumped through a 21 gauge needle to 1000 mL of a stirred solution containing 2% of calcium chloride in deionized distilled water at 25° C. as depicted in FIG. 6. The resultant mixture was stirred for about one additional hour at about 25° C. The mixture was filtered and beads washed with 3×1750 mL volumes of distilled water to remove excess calcium chloride. The resulting beads have structure because of the cross-linking, and the negative carboxyl groups that are not involved in cross links are neutralized by sodium and/or calcium counterions present in the diffuse double layer.

The dried beads from above were immersed in 2000 mL of 2.5% W/V propranolol hydrochloride solution and stirred for 3 days at about 25° C. The resulting drug-loaded beads were harvested by filtration, and washed with multiple 1250 mL volumes of distilled water until the drug concentration became negligible, (e.g., four washings). The drug-loaded spheres were air-dried at approximately 25° C.

### 6.2 Example 2

Example 2 describes the results of equilibrium binding studies involving the exemplary electrolytic drug propranolol hydrochloride and exemplary ion-exchange matrix including sodium alginate, xanthan gum, and gellan gum.

The studies were performed with a two compartment plexiglass dialysis cell (Hollenbeck laboratory) and having a cellulose membrane (molecular weight cutoff of 6000 Daltons) Bel-Art Products (Pequannock, N.J.) placed between the two cell compartments. For the sodium alginate studies, one compartment (“the drug compartment”) was charged with 15 mL of a 0/97×10⁻² molar solution of propranolol hydrochloride in deionized water, while the other compartment (“the polymer compartment”) was charged with 15 mL of a 0.0877% W/V solution of the sodium alginate in deionized distilled water. The dialysis cell was shaken at 80 RPM in a thermostatic water bath at 25° C. until equilibrium was reached (30 h). The solution was removed from the drug compartment and the concentrations of free drug and polymer-bound drug measured by high performance liquid chromatography (“HPLC”).

Results of the binding studies of propanolol hydrochloride using sodium alginate are shown in Table 1.

TABLE 1
Binding data for propranolol with sodium alginate.^{a}

| | | | Amount | | |
|---|---|---|---|---|---|
| | | | bound per | | |
| Total drug | Free drug | Bound drug | gram of | | |
| concentration | concentration | concentration | polymer (M × | | |
| (M × 10⁴) | (M × 10⁴) | (M × 10⁴) | 10⁴) | % Bound | (l/g) |
| 50.70 | 39.36 | 11.34 | 12.93 | 22.36 | 0.33 |
| 41.20 | 30.35 | 10.84 | 12.37 | 26.33 | 0.41 |
| 30.40 | 20.32 | 10.08 | 11.49 | 33.16 | 0.57 |
| 24.86 | 15.45 | 9.41 | 10.73 | 37.84 | 0.60 |
| 19.88 | 11.28 | 8.60 | 9.81 | 43.28 | 0.87 |
| 14.91 | 7.36 | 7.55 | 8.61 | 50.63 | 1.17 |
| 9.94 | 4.10 | 5.83 | 6.66 | 58.75 | 1.62 |
| 8.95 | 3.57 | 5.37 | 6.12 | 60.02 | 1.71 |
| 7.95 | 2.49 | 5.46 | 6.23 | 68.66 | 2.49 |
| 6.96 | 1.82 | 5.14 | 5.86 | 73.80 | 3.21 |
| 5.96 | 1.43 | 4.53 | 5.16 | 75.94 | 3.60 |
| 5.47 | 1.22 | 4.25 | 4.84 | 77.68 | 3.97 |
| 4.97 | 0.94 | 4.03 | 4.59 | 81.06 | 4.88 |

^{a}The concentration of sodium alginate in the polymer compartment for all studies was 0.877 g/L.

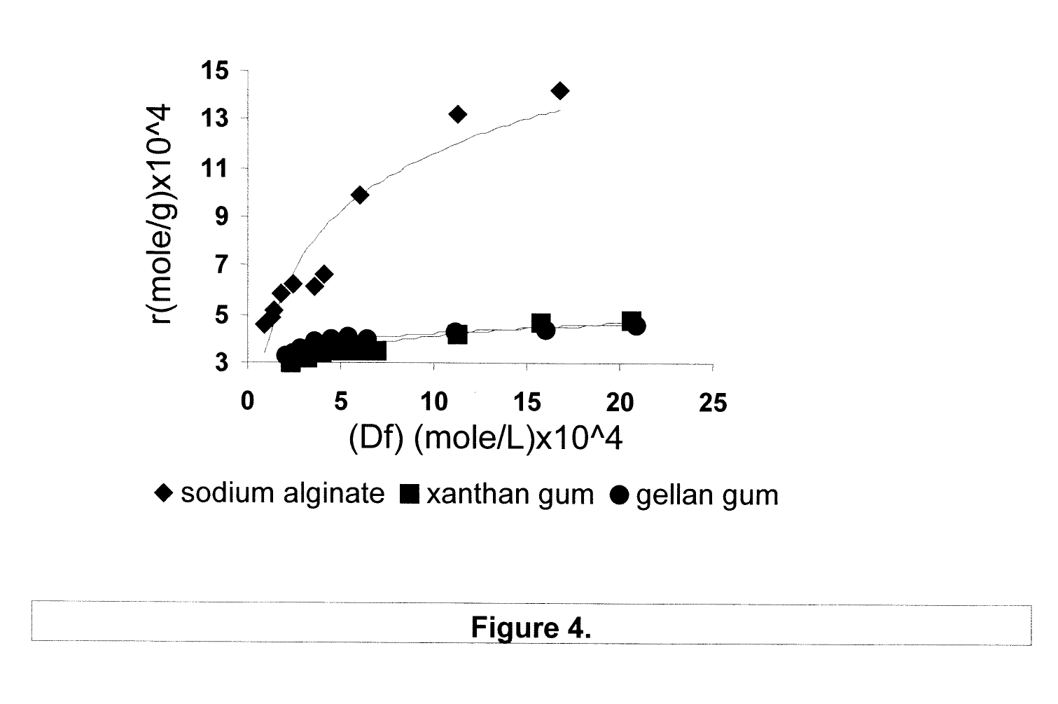

The binding isotherm of propranolol hydrochloride with sodium alginate is shown in FIG. 4 as a plot of r versus D_{f}, where r is the moles of drug bound per gram of polymer and D_{f} is the molar free drug concentration in the system. FIG. 4 also includes data for the cation-exchange matrices gum and gellan gum. The resultant Langmuir-type drug-polymer interaction isotherms indicate that the extent of binding increases with free drug concentration for all three cation-exchange matrices, and the binding is capacity limited.

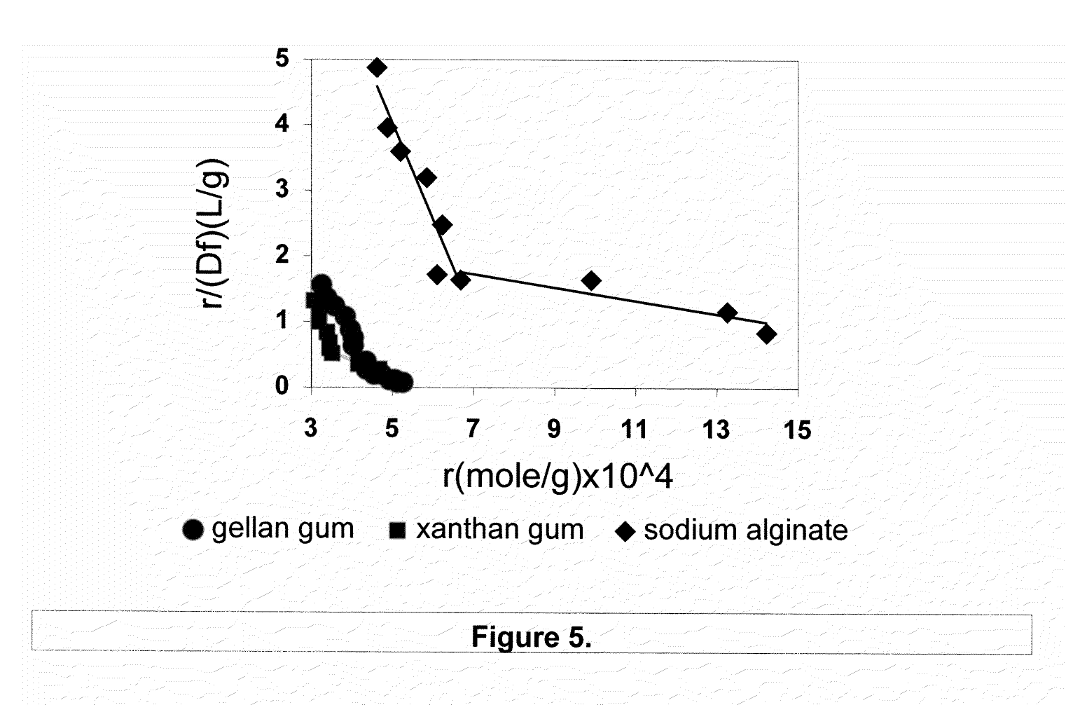

FIG. 5 shows a Scatchard plot of the binding data of r/(D_{f}) versus r for the propranolol hydrochloride with the sodium alginate, xanthan gum or gellan gum. In typical complexation or site specific binding studies with only one type of binding site, the Scatchard plot would be a single straight line where the slope is the association constant (K) and the intercept at the abscissa is equal to the binding capacity (a). FIG. 5 shows that the plots are not single straight lines in the concentration ranges that were studied. Inflection in these plots normally is taken to indicate competitive interactions, the existence of more than one type of binding site, or both. However, in this case non-linearity is not surprising given that the association is non-specific neutralization in the diffuse double layer.

It does appear, particularly for sodium alginate, that the data could be modeled with two different “binding sites” (e.g., two straight lines). The slopes and the intercepts of the initial portion of each Scatchard plot were determined by linear regression. The residual method was used to determine the second affinity constant (K₂) and binding capacity (n₂) in the high drug concentration range. Using this model, the total drug binding capacity (n_{T}) is then equal to the sum of n₁ and n₂. This model would be consistent with the location of some bound drug in the Stem layer of the DDL and the remainder in the more diffuse region referred to as the Guoy-Chapman layer (these layers are described in Remington: The Science and Practice of Pharmacy, 20^{th} ed., Alfonso Gennaro, Lippincott Williams & Wilkins (2000) at chapter 21: Colloidal Dispersions at page 300; Physical Pharmacy, 4^{th} ed. Alfred Martin, Lea & Febiger (1993) at chapter 15: Colloids, at page 405.) These parameter estimates are provided in Table 2.

TABLE 2
Binding capacities and affinity constants for the interaction of propranolol
with the cation-exchange matrix matrices sodium alginate, xanthan gum,
and gellan gum.

| | Binding Capacity, mole/g × 10⁴ | | | Affinity | |
|---|---|---|---|---|---|
| | | | Total | Constant, | |
| | 1^{st} Binding | 2^{nd} Binding | Binding | (M × 10⁴)⁻¹ | |
| Polymer | Capacity n1 | Capacity n2 | Capacity n_{T} | K₁ | K₂ |
| Sodium | 7.74 | 7.08 | 14.82 | 1.46 | 0.04 |
| Alginate | | | | | |
| Xanthan | 3.86 | 1.09 | 4.95 | 1.64 | — |
| Gum | | | | | |
| Gellan Gum | 4.76 | 0.58 | 5.34 | 1.04 | — |

The results in Table 2 show that the first affinity constants (K₁) are similar for different ion-exchange matrices in the low propranolol hydrochloride concentration range. The results also show that sodium alginate has the highest total binding capacities at both high and low propranolol hydrochloride concentration ranges of the three ion-exchange matrices studied.

The results of the binding studies demonstrate the electrostatic association of drug and polymers that are ion-exchange matrices, hence the feasibility of a dosage form comprising ion-exchange matrix drug complexes. The results of the binding studies also provide some basic information for formulating ion-exchange matrix drug complexes. For example, a solution containing a 60 mg (2.02×10⁻⁴ mole) dose of propranolol hydrochloride (MW=295.84) will require an ion-exchange matrix having sufficient capacity to associate with at least 1.82×10⁴ moles of the drug in order to meet the arbitrarily selected limit of 10% free drug concentration. For sodium alginate, the maximum binding capacity is 1.48 mmoles per gram (Table 2), so a quantity of about 123 mg of sodium alginate is expected to be sufficient to accommodate the dose of drug under maximum binding conditions. This appears to be a reasonable amount, given the fact that a dosing volume of up to 15 ml would not be problematic (e.g., a colloid concentration of 0.82% W/V).

### 6.3 Example 3

### Coating the Ion-Exchange Matrix Drug Complexes

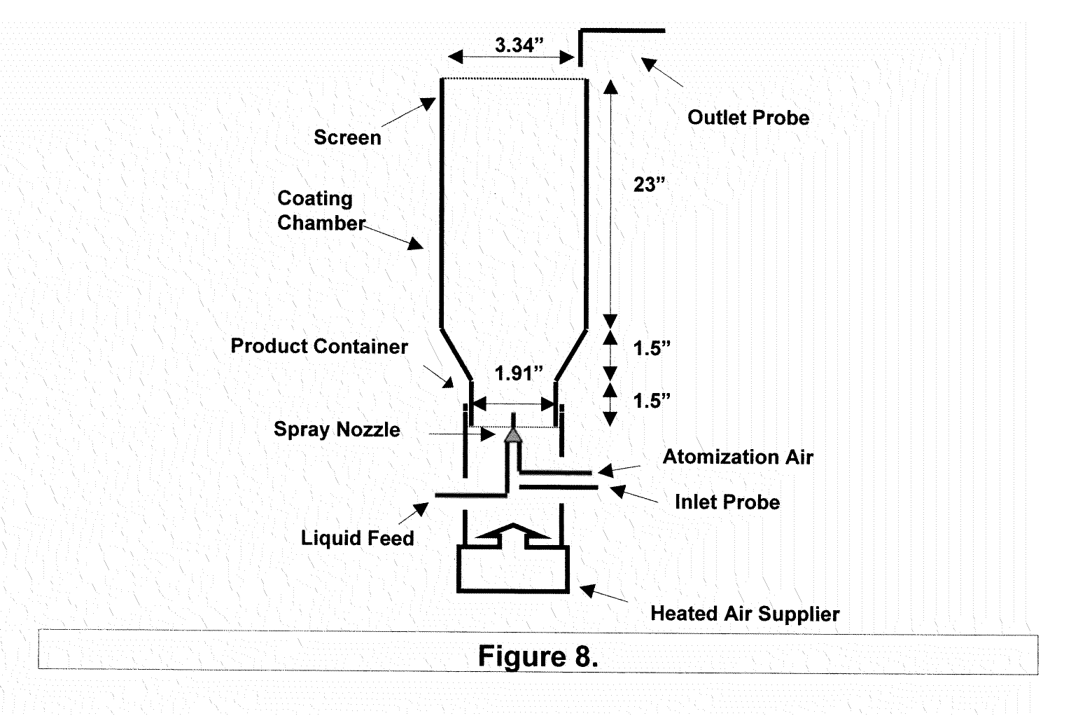

Coating of the ion-exchange matrix drug complexes was performed using the fluid bed coater depicted in FIG. 8, which is useful for processing solids in the 5 to 30 g range. Typically, about 8 g of ion-exchange matrix drug complex was charged to the fluid bed coater. The inlet temperature was set to about 40° C. and the bed temperature was set to about 30° C. An aqueous dispersion containing Eudragit®RS 30 D (8.34 g) and triethly citrate (1.67) was prepared, and the dispersion was applied at a spray rate of 0.97 ml/min and at an atomization air pressure of about 30 psig. After application was completed, the coated particles were allowed to dry under flowing air in the fluid bed coater. The dried coated particles typically contained about 20-30% by weight of coating based on the total weight of applied coating and ion-exchange matrix drug complex.

### 6.4 Example 4

### In Vitro Testing

Example 4 describes the results of an in vitro feasibility study showing that both the integrity of the coating and the integrity of the ion-exchange matrix in the dispersed phase (e.g., the beads) is maintained. The salt form of propanolol (propanolol HCl) was employed as a model active ingredient. Propanolol is a weakly basic drug, with a reported pKa of 9.4. At pH values of 7.4 or less, propranolol is protonated and positively charged.

In vitro testing was done utilizing the traditional USP Apparatus 2 at 37° C., with a stirring speed of 100 RPM. Ca-alginate drug-loaded spheres coated with Eudragit®RS 30 D and equivalent to 160 mg of propranolol hydrochloride were dispersed using hand shaking in a suspension medium (15 mL) containing a hydrophilic colloid useful both as a polyelectrolyte and suspending agent, and the suspension was added directly to 500 mL of simulated gastric fluid (SGF (i.e., the release medium). After 2 h of agitation, a sufficient quantity of tribasic sodium phosphate was then added to change the pH to 7.5, thus effectively changing the medium to simulated intestinal fluid for the remaining 10 h.

Eight suspensions containing different dispersion mediums were prepared and tested as described above. The hydrophilic colloid polyelectrolytes used in the dispersion media included hydroxypropylmethylcellulose (“HPMC”) (F₁ and F₂), xanthan gum (F₃ and F₄), propylene glycol alginate (F₅), chitosan (F₆) and gelatin (F₇). The ionic content, pH; viscosity of the dispersion medium; and percentage of drug released into the dispersion medium were measured and the results provide in Table 3. Ionic content was the sum of the diffusible counter cations K⁺, Na⁺ and Ca²⁺ present in the hydrophilic colloids; pH indicates the extent of drug release from the core, i.e., the lower the pH of the dispersion medium, the greater the extent of drug release; and viscosity is indicative of the effectiveness of the hydrophilic colloid as a suspending agent. The extent of drug release into the suspension medium is also provided in Table 3.

TABLE 3
Formulations, properties of the dispersion medium and extent of propanolol
hydrochloride release from calcium alginate coated beads dispersed in
media containing various hydophilic colloids.

| | | Ionic | | | | Drug |
|---|---|---|---|---|---|---|
| | | Content | | Viscosity (cP) | | Leaching, |
| Formulation | Components | (ppm)^{a} | pH | 0.6 RPM | 60 RPM | %^{c} |
| F₁ | 0.8% HPMC | 0^{b} | 6.11 | 1559.7 | 879.8 | 2.13 ± 0.22 |
| F₂ | 0.8% HPMC | 0^{b} | 5.84 | 1559.7 | 947.8 | 0.00 ± 0.00 |
| | Sucrose + | | | | | |
| | Preservatives | | | | | |
| F₃ | 0.5% Xanthan | 13.00 | 5.54 | 3399.3 | 162.0 | 7.95 ± 0.42 |
| | gum | | | | | |
| F₄ | 0.2% Xanthan gum | 5.20 | 5.00 | 1249.7 | 113.5 | 4.62 ± 0.07 |
| F₅ | 1.95% | 334.63 | 3.34 | 1349.7 | 415.9 | 8.19 ± 0.66 |
| | Propylene | | | | | |
| | Glycol | | | | | |
| | Alginate | | | | | |
| F₆ | 4% Chitosan | 4.79 | 5.57 | | | 4.34 ± 0.13 |
| F₇ | 1% Gelatin | 17.69 | 5.25 | | | 9.02 ± 0.17 |
| F₈ | Deionized | 0.00 | 6.40 | | | 2.02 ± 0.09 |
| | water | | | | | |

^{a}Ion content is the sum of K+, Na+ and Ca2+.
^{b}Actual value from calibration curve is −0.61.
^{c}mean ± SD for 3 replicates, % leaching = free drug content in the medium/total drug.
^{d}10% w/v of sucrose was added as a flavoring agent, and the combination 0.2% w/v of methylparaben and 0.01% w/v of propylparaben was added as a preservative.

The results in Table 3 show that the concentration of diffusible counterions in the dispersion medium strongly influences the extent of release of the drug ion from the ion-exchange matrix drug complex. The results indicate that additives such as suspending agents must contain a low content of diffusible counterions in order to minimize the extent of release of ionic drug.

The results in Table 3 also show that the strongly anionic polyelectrolyte propylene glycol alginate (F₅) provided a suspension with the highest extent of release of the cationic drug propanolol HCl. This result indicates that additives such as polyelectrolytic suspending agents that have a charge opposite that of the drug ion in the ion-exchange matrix drug complex will affect the levels of free drug concentration in the dispersion medium.

### 6.5 Example 5

Example 5 describes a method for making core alginate beads loaded with the drug albuterol.

The formulation for alginic acids beads is depicted in Table 4.

Alginic Acid Bead Formulation

| | | | |
|---|---|---|---|
| Albuterol | 5.0% | 12.5 | g |
| Alginic Acid | 5.0% | 12.5 | g |
| Lactose monohydrate (20%) | 20.0% | 50 | g |
| Avicel PH 101 | 70.0% | 175 | g |
| | 100% | 250 | g |

A target drug loading of 5% was used, with equal quantities of albuterol and alginic acid. The albuterol base and the alginic acid powders were placed in a beaker, and 100 mL of water was added. The mixture was agitated using a magnetic stir bar and stirrer. An additional 50 mL of water was added to wash down the sides of the beaker, and agitation was continued until a homogeneous gel was formed.

The microcrystalline cellulose and lactose was placed into the bowl of a low shear mixer and a pre-blend was effected using a spatula. The drug-containing gel was then added while mixing at low speed. The entire quantity of gel was added, to produce a wet mass. The resulting material was extruded using a radial screen attachment with 1 mm screen size. The extrudate was then spheronized at 750 rpm for approximately 30 sec, and then dried overnight in a convection oven at 45° C.

### 6.6 Example 6

Example 6 describes a method for making methacrylic acid (Carbopol) core beads loaded with the drug albuterol.

The formulation for the core methacrylic acid (Carbopol) beads is presented in Table 5.

TABLE 5
Carbopol Bead Formulation

| | | | |
|---|---|---|---|
| Albuterol | 2.5% | 6.25 | g |
| Carbopol 974P | 2.5% | 6.25 | g |
| Lactose monohydrate (20%) | 20.0% | 50 | g |
| Avicel PH 101 | 75.0% | 187.5 | g |
| | 100% | 250 | g |

A target drug loading of 2.5% was used with equal quantities of albuterol and alginic acid. Carbopol 974P was employed based on information from the supplier that this grade had greater cross-linking and was designed for oral use.

The microcrystalline cellulose and lactose were placed into the bowl of the low shear mixer and a pre-blend was effected using a spatula. The drug-containing gel was then added while mixing at low speed. The entire quantity of gel was added, plus an additional 10 mL of water, producing a wet mass. The resulting material was extruded using the radial screen attachment with 1 mm screen size. The extrudate was then spheronized at 750 rpm for approximately 30 sec, and then dried overnight in a convection oven at 45° C.

### 6.7 Example 7

Example 7 describes the results of studies involving exemplary coated alginate beads.

The beads were coated in the mini-fluid bed processor with Eudragit RS30D, including triethylcitrate as a plasticizer at a level of 10% of coating solids. The final product was targeted for a coating level of 35%, producing an evenly coated bead with a smooth, glossy surface.

As a crude assessment of physical stability, a 1 g sample of the coated beads was placed in 10 mL of water. Typically, when dispersed in water, the beads will absorb water and rupture within a few hours, producing a paste like substance. Although the dispersion medium turned a little “hazy,” these beads maintained their integrity for over 4 days. A sample of the suspended beads was placed on a microscope slide and excess water was removed with an absorbent swab. Observed under a microscope, some water is still present in a pendular state, holding the beads together through capillary forces; however, the beads are clearly intact, and even the shiny surface of the coating is still evident. The alginate-based beads retain their spherical integrity in water and the functional coating appears to be intact also.

Similarly, the Carbopol beads remained intact when exposed to water; however, the behavior was slightly different. Observed over a 75 minute time period, these beads eventually swell in water, and the coating loses its integrity. However, even though the coating loses its integrity, each swollen bead still appears to remain intact. Even after agitation, the discrete character of each bead is retained.

### 6.8 Example 8

Example 8 describes the results of studies conducted to evaluate the physical stability and the controlled release of albuterol from coated beads.

The following assay determinations have been done by HPLC using a method based on that in the USP for Albuterol Tablets. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 1 mL samples at the following time points: 5, 30, 60, 90 minutes; 4, 8, 12, 16, 20, 24 hrs. The 5 minute point is included to determine the quantity of albuterol that is “immediate” release. This early time point is determines the free drug concentration in the dispersion medium of a suspension dosage form as well as the effectiveness of coating when the sample is dry beads.

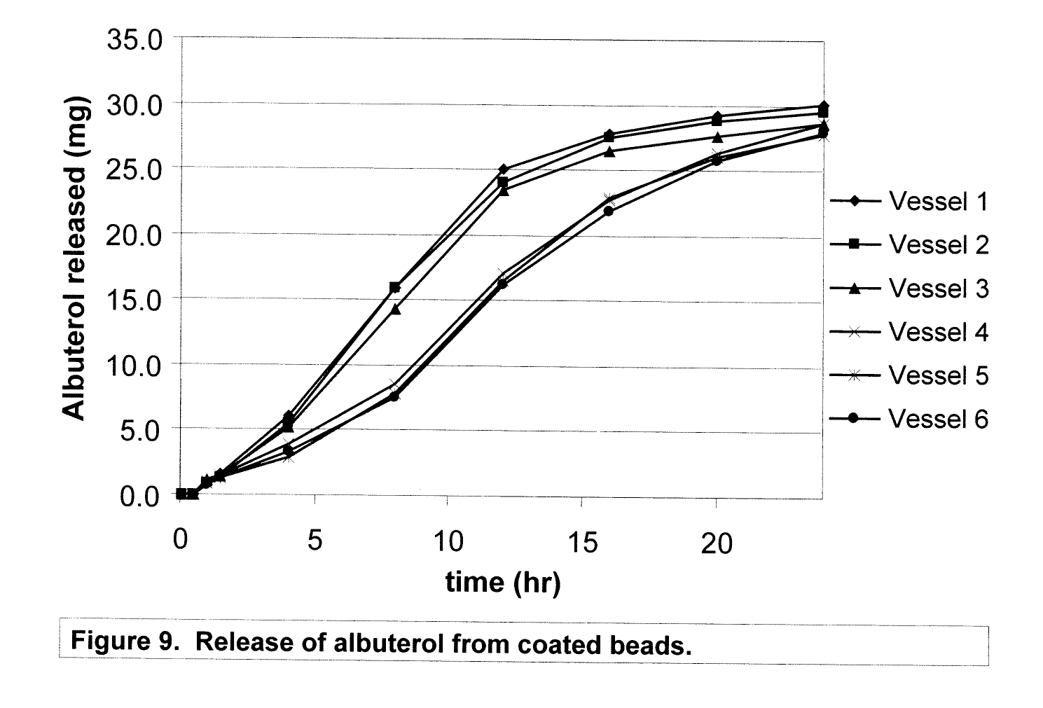

The two products represented in Tables 4 and 5, each of which was coated with 35% Eudragit RS30D, were examined for albuterol release, and the results are presented in FIG. 9. Based on the formulation and the target coating weight, each sample tested contains 32.5 mg of albuterol.

Both products demonstrate sustained drug release across a 24 hour period. The carbopol-based formulation presents a profile that is nearly “zero” order, and this product releases drug more slowly than the alginate-based formulation.

When suspensions in Syrup NF (i.e., 65% W/W sucrose) were analyzed, no albuterol peaks were observed on the initial chromatograms, indicating that essentially all the drug remained inside the beads.

Additional samples were prepared for release testing as suspensions containing approximately 50 mg of albuterol in 10 mL liquid. For the alginate-based product, a 1.5 g quantity of beads was used in each system, while a 3.0 g quantity of the less potent carbopol-based beads was used. Three vehicles were used: water, syrup, and a 50:50 mixture (“50% syrup”). The preparations were allowed to stand for 48 hours before testing.

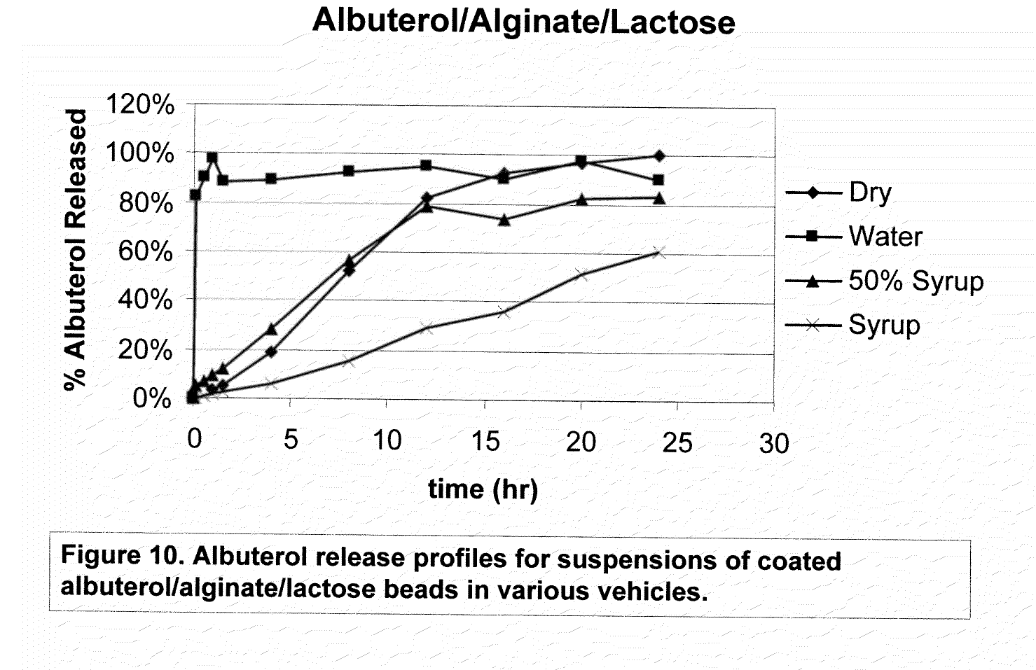

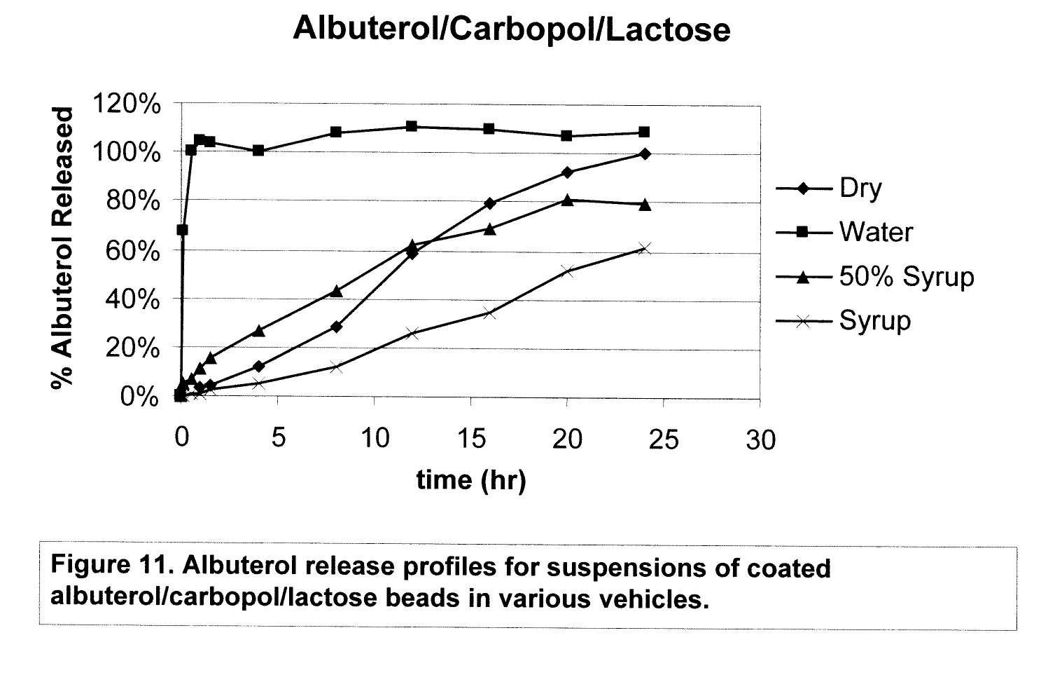

The dissolution results have been translated into % released, with a potency estimate based on the amounts released at 24 hr in FIG. 9. The result for each suspension product was then plotted against the appropriate average value obtained for the dry beads in FIG. 5. Results for the carbopol-based product are seen in FIG. 10, while those for the alginate-based product are in FIG. 11.

The behavior of the two formulations is quite similar. In each case, beads suspended in water did not provide any control over albuterol release. Release from the same beads suspended in syrup is slower than that seen for dry beads, while the suspensions in 50% syrup come closer to emulating dry beads.

Based on the results, there is an effect of the syrup vehicle on the permeability of the Eudragit coating. Without being limited by theory, this is most likely due to the high osmotic pressure of syrup itself and commensurate low water activity in the concentrated sucrose solution.

### 6.9 Example 9

Example 9 describes the results of studies conducted to evaluate the impact of water activity in the dispersion medium on the performance, physical stability and controlled release of albuterol from coated beads.

The impact of water activity in the vehicle on the performance of products made with lactose-containing beads was examined using the alginate system. Three systems were examined as vehicles, and suspensions were prepared and stored at room temperature for 7 days.

First 50% W/W Sucrose in water was examined. Stability has been demonstrated in the sucrose-containing vehicles, and based on previous results, albuterol release from beads suspended in 40-50% sucrose vehicles did not differ dramatically from what was seen from the coated beads alone. The other two systems examined were methylcellulose dispersions. Results for these products are presented in FIG. 8.

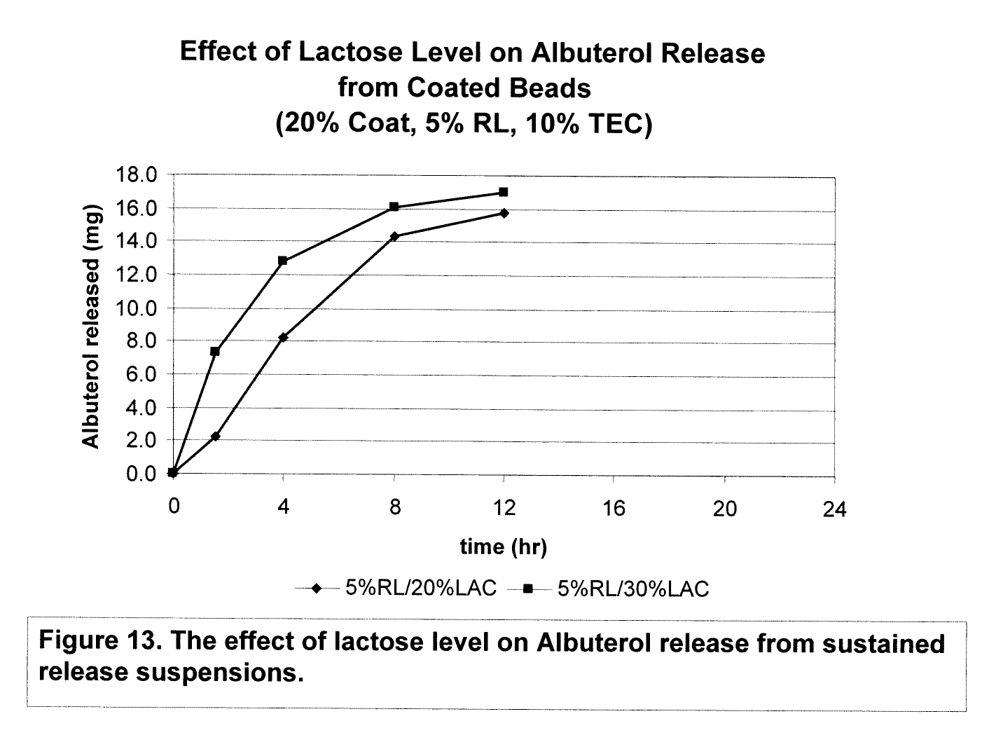

In the suspension in 50% W/W sucrose vehicle, compared to the profile for the coated beads, release is somewhat slower. This effect has been seen consistently, without being limited by theory, Applicants believe that additional curing takes place in the hyper-osmotic vehicle. Residual water in the coating would diffuse into the concentrated sucrose solution in the same way that a red blood cell shrinks in an hypertonic environment. Alginate bead formulations of albuterol have been prepared with lactose concentrations of 10, 20, 30, and 40% W/W. The level of lactose has the potential to effect both release rate and stability. For example, in FIG. 13, albuterol release is faster for the product with 30% lactose when compared to the one with the 20% level.

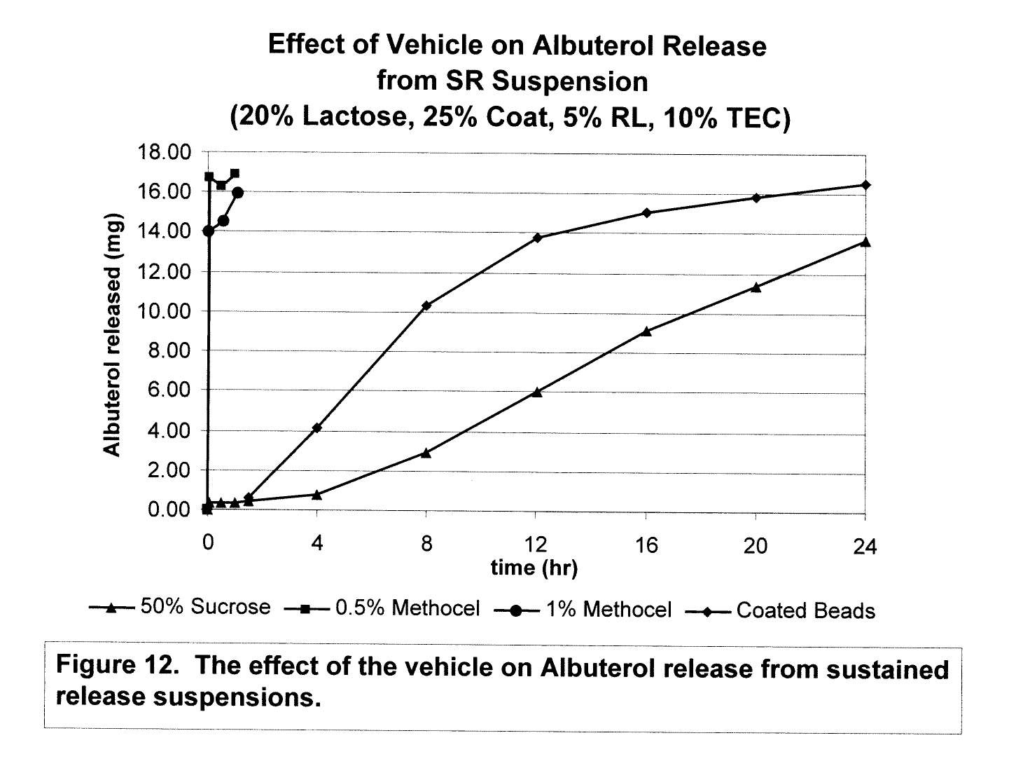

The coating system used for the beads in FIGS. 12 and 13, is based on a binary combination of Eudragit RL and Eudragit RS; the RL system is more permeable than the RS system. Based on these results, it is clear that the ratio of these polymers can be adjusted to manipulate the characteristics of the internal phase of the suspension.

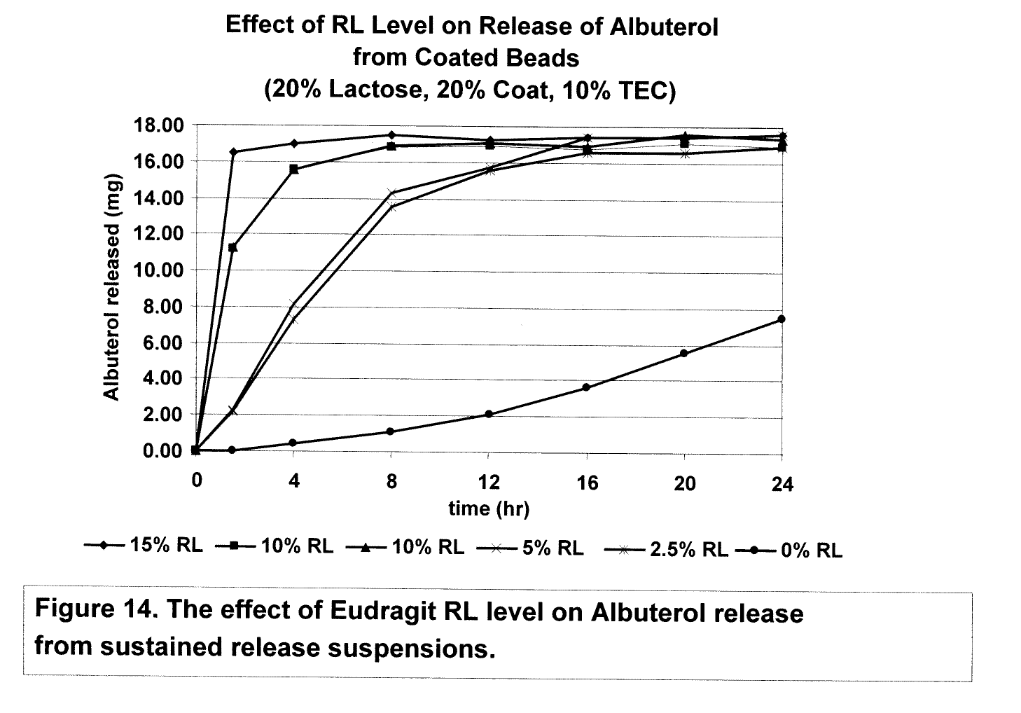

Presented in FIG. 14 are release profiles for beads coated with various ratios of the polymers, at the 20% overall coating level.

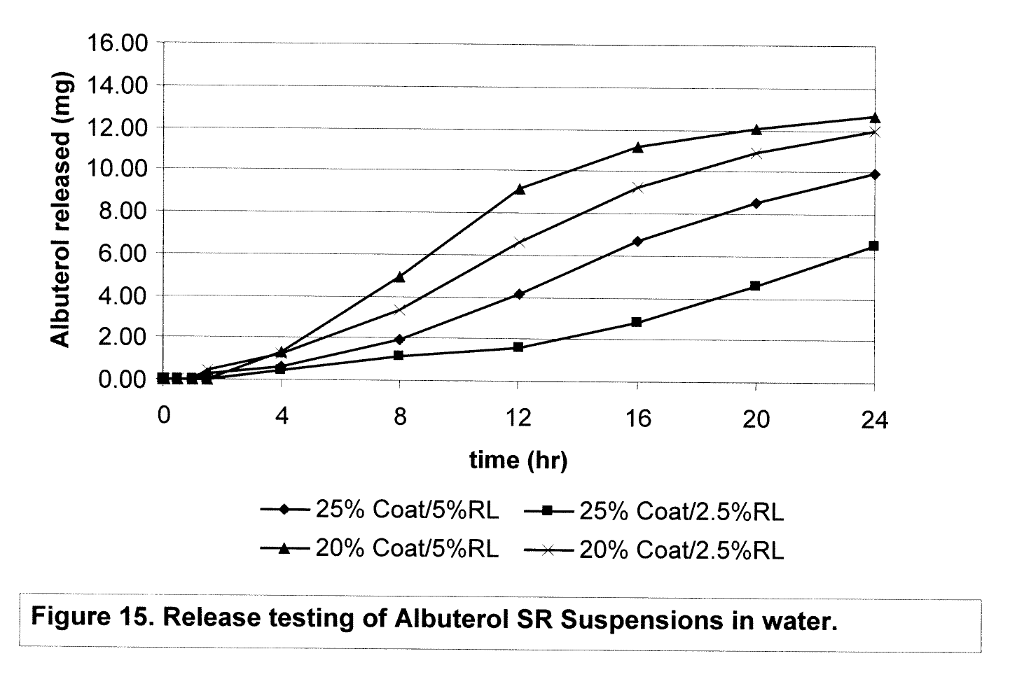

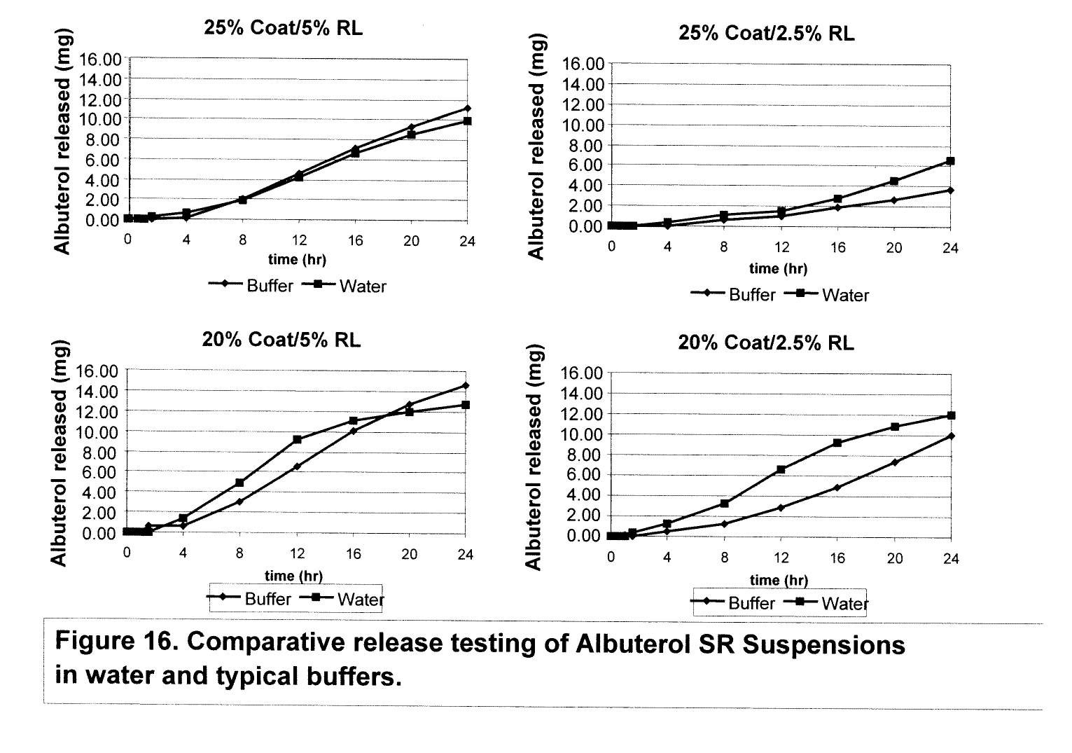

Presented in FIGS. 15 and 16 are release profiles for beads coated with various ratios of the polymers in water as well as typical buffers.

Design optimization of sustained release suspensions similar to this one, in terms of physical stability and controlled release, requires as a minimum: consideration of the lactose level in the bead formulation; the ratio of permeable (Eudragit RL) to non-permeable (Eudragit RS) polymer in the functional coating system; the plasticizer level; and the concentration of sucrose in the vehicle. Such variations will be apparent to one of skill in the art.

### 6.10 Example 10

Example 10 describes the results of the studies conducted to evaluate the impact of sucrose concentration in the dispersion medium on physical stability and controlled release profile of the extended release composition.

The experiment was carried out with 16 mg of albuterol bound to the alginic acid resin, with 20% coating, and 40%, 50%, 60% or 65% W/W of sucrose in the dispersion medium. First, the alginate beads were loaded with the drug albuterol, where 5% of albuterol and 5% of alginic acid were used. Albuterol and alginic acid powders were placed in a beaker, 100 ml of water was added, and the mixture was stirred until a homogenous gel was formed. Then, 70% microcrystalline cellulose and 20% lactose were placed into a bowl of a low shear mixer and pre-blended with a spatula. Then, the albuterol-alginate gel was incrementally added to the microcrystalline cellulose and lactose blend and mixed to produce a wet mass. Additional 50 ml of water was added to the beaker to wash down its sides and added to the wet mass during its blending, and the resulting wet mass was transferred into a polyethylene bag. Then, the resulting wet mass material was extruded using a dome screen attachment with 0.8 mm screen size at 55 rpm, with the wet mass being gradually added into the extruder. Subsequently, the extrudate was spheronized at 750 rpm for approximately 30 sec, and then dried overnight in a convection oven at 45° C. The beads of appropriate size (material that passed through 16 mesh screens and retained on 20 mesh screens) were selected. The beads were then coated in the mini Fluid Bed coater with Eudragit RS30D (30% solids) with 10% of Triethylcitrate as a plasticizer. The coating level of a final product was approximately 20%. Finally, coated beads were suspended in a dispersion medium containing 40%, 50%, 60% or 65% W/W of sucrose, and such suspensions were prepared to contain 16 mg of albuterol. The samples were stored at room temperature for 1 week prior to analysis. Then, assay determinations were made by HPLC using a method based on that in the USP for Albuterol tablets. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 1 mL samples at the following time points: 5, 30, 60, 90 minutes; 4, 8, 12, 16, 20, 24 hours.

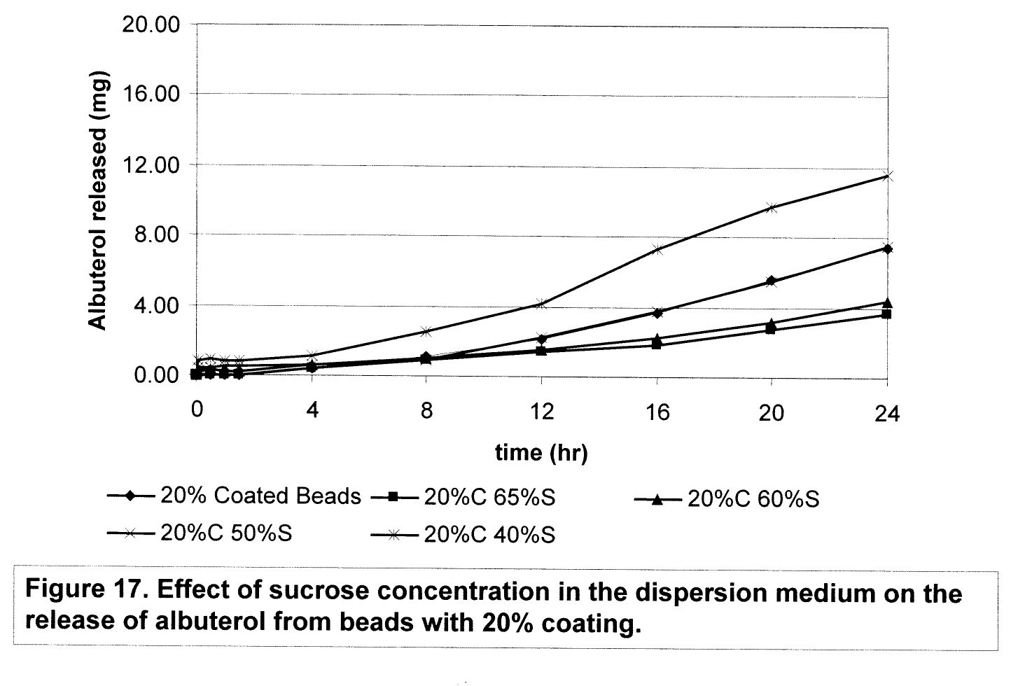

FIG. 17 shows the results of the experiment described above. FIG. 17 demonstrates that release of the drug albuterol from beads suspended in 50%, 60% and 65% W/W of sucrose is slower than from beads suspended in 40% W/W of sucrose. The results of the experiment presented in FIG. 17 reveal that use of 50%, 60% or 65% W/W of a highly hydrated excipient, such as sucrose, in the dispersion medium unexpectedly achieves a stable liquid form controlled release drug composition and a slower drug release profile than use of 40% W/W of a highly hydrated excipient. Thus, use of 50% to 70% W/W of a highly hydrated excipient in the dispersion medium is surprisingly more effective than use of 40% W/W of a highly hydrated excipient at achieving a preferred release profile of a drug in a liquid form controlled release drug composition.

### 6.11 Example 11

Example 11 describes a method of manufacture of “Formulation X.” “Formulation X” is a liquid dispersion of ER coated pellets in syrup intended for the treatment of cough, cold, and allergy symptoms. Formulation X contains 15 mg hydrocodone bitartrate (HC, a centrally-acting antitussive), 120 mg pseudoephedrine hydrochloride (PSE, a sympathomimetic nasal decongestant), and 8 mg chlorpheniramine maleate (CPM, an anti-histamine) in combination per adult dosage (5 ml). In this formulation, the salt forms of the drugs have been used. This formulation was sorted into (4 oz) unit-of-use containers upon manufacture.

Table 6 presents a table outlining an example quantitative composition of Formulation X IR/ER liquid dispersion of extended release pellets in syrup, expressed on a weight basis, in terms of a single 5 ml (6.55 g) dose.

TABLE 6
Quantitative Composition of Formulation X
IR/ER Liquid Dispersion of Extended Release Pellets in Syrup

| Component | Percent Weight |
|---|---|
| Chlorpheniramine Maleate | 0.1382% |
| Pseudoephedrine HCl | 2.073% |
| Hydrocodone Bitartrate | 0.2591% |
| Lactose monohydrate | 1.252% |
| Sodium Alginate | 0.2504% |
| Microcrystalline Cellulose | 8.649% |
| Eudragit RS30D | 3.222% |
| Eudragit RL30D | 0.1342% |
| Triethylcitrate | 0.3504% |
| Talc | 1.043% |
| Titanium Dioxide | 0.2068% |
| FD&C Red #40 Lake | 0.0827% |
| Artificial Strawberry Flavor | 0.3309% |
| Bitter Masking Flavor | 0.2482% |
| Sucralose | 0.08273% |
| Sucrose, NF | 52.86% |
| Propylparaben | 0.01254% |
| Purified Water | 28.81% |
| Total | 100.00% |

The ratio of API concentration in the IR syrup compared to the ER pellet has been designed to provide serum drug bioavailability for 12 hours in a manner that is BE to the immediate release drugs that are currently in the market.

The ratio of API concentration in the IR syrup compared to the ER pellet has been designed to provide serum drug bioavailability for 12 hours in a manner that is BE to the immediate release drugs that are currently in the market.

### 6.11.1 Manufacture of Core Pellets for Formulation X

The ER component of Formulation X comprises core pellets. A method for preparing core pellets is described below.

0.207 kg of chlorpheniramine maleate, 4.138 kg of pseudoephedrine hydrochloride, 0.3879 kg of hydrocodone bitartrate, 2.500 kg of lactose monohydrate, 0.5000 kg of sodium alginate and 17.27 kg of microcrystalline cellulose were placed in combination in a high shear mixer. The mixer was operated for 5 minutes with the impeller at 200 RPM and the chopper at 1500 RPM. Subsequent to this powder blending, approximately 10.00 kg of purified water was pumped into the mixer at a rate of approximately 1 L/minute while operating the impeller at 200 RPM and without using the chopper. After all the water was added, wet granulation was continued for 1 minute with the impeller at 200 RPM and the chopper at 1500 RPM. After discharge, the wet mass was left to sit in the open bags for a minimum of 30 minutes.

The resulting wet mass was then extruded using a single screw dome-type extruder with 0.7 mm screen, operating at 45 RPM. Extrusion was continued until the entire quantity of wet mass was processed.

The formation of pellets was accomplished as repetitive batch processes using a spheronizer with a disk having a 3×3 mm truncated pattern operating at approximately 1000 rpm. Nine batches of approximately 3.8 kg each were used with each spheronization run lasting approximately 90 seconds.

The spheronized material was placed in a fluid bed dryer. The beads were dried with an initial inlet air temperature of 60° C., and total air volume of 1500 cubic feet per minute. Subsequent in-process adjustment of inlet air temperature and volume was made to maintain proper fluidization and a product temperature in the range of 50-60° C. Drying was continued until the beads achieved a moisture content of 2% or less. The fluid bed dryer was operated in the cooling mode for sufficient time to bring the product to room temperature, and then the dry beads were discharged.

Sizing of the dry beads was accomplished using an automatic sieve shaker with #22 and #34 screens operating at 1200 RPM. Core pellets passing through the #22 screen and retained on the #34 screen were considered acceptable.

### 6.11.2 Coating of the Pellets for Formulation X

A method for coating the core pellets of Formulation X is described below.

8.65 kg of ammonio methacrylate copolymer type B liquid dispersion (Eudragit RS30D) was added through a #20 screen into a stainless steel vessel. 0.7774 kg of ammonio methacrylate copolymer type A liquid dispersion (Eudragit RL30D) was then added through a #20 screen to the same container. The combination of liquid dispersion was mixed using a propeller mixer at approximately 1000 rpm. In a separate container, 0.6087 kg of triethyl citrate and 10.92 kg of purified water were combined. This combination of liquids was also mixed using a propeller mixer at an rpm sufficient to produce a vortex without introducing air into the liquid. While continuing to mix, 1.812 kg of talc was added to the triethyl citrate and water mixture. After mixing for 5 minutes, the stirrer was stopped and removed. The mixture of triethyl citrate, talc and water was transferred into the container with the methacrylate copolymer dispersions with agitation continuing. Sufficient agitation of this coating system was continued throughout the coating operation to maintain a uniform dispersion.

Coating of the core pellets was performed in a Glatt GPCG 30 fluid bed processor with a 12 inch Wurster product container, using a D base plate with a 250 μm screen. The column height was set at approximately 2.5 inches from the bottom and a nozzle with a 1 mm opening was employed. 21.75 kg of core beads was placed in the fluid bed processor with initial parameter settings: inlet air temperature target range, 40-50° C.; product temperature target range, 27-32° C., atomization air pressure target range, 1.5-2.5 bar; air volume target range, 250-450 cfm; total air volume, 1500 cfm; filter bag shake interval, 5 seconds every 240 seconds. In process adjustments were made to maintain processing conditions within range. Initial spraying of the coating dispersion was accomplished at a rate of 20 g/minute; as processing continues, the spray rate can eventually be elevated to 100 g/minute.

After all the coating dispersion was applied, the fluid bed processor was operated in cool mode to bring the coated beads to room temperature, and then they were discharged. Subsequently, the coated beads and 75.00 g of talc were blended for 5 minutes and discharged.

Curing of the coated pellets was accomplished in a convection oven. The coated pellets were distributed into trays and placed into the oven. The oven temperature was set for 55° C., and the beads were maintained in the oven for approximately 16 hours. After this time period, the oven was turned off and the pellets were allowed to cool to room temperature.

Sizing of the coated beads was accomplished using an automatic sieve shaker with #16 and #34 screens operating at 1200 RPM. Coated pellets passing through the #16 screen and retained on the #34 screen were considered acceptable.

### 6.11.3 Preparing a Vehicle for the Core Pellets of Formulation X

The IR component of Formulation X comprises a liquid, also called a “vehicle” or “vehicle syrup” for Formulation X. A method for making a vehicle syrup is described below.

25.09 kg of purified water was placed in a jacketed stainless steel vessel of sufficient capacity to hold 60 L. A circulating water temperature control system was used to control the temperature of the liquid throughout the operation and a propeller mixer was used to produce agitation. A cover was employed to limit evaporation of water. The water was heated to approximately 70° C. while mixing at a speed such that a vortex was produced without introduction of air into the liquid. 0.01080 kg of propylparaben passed through a #30 screen was added to the water and mixing was continued until the propylparaben completely dissolved. After dissolution of the propylparaben, the temperature control unit was set to 50° C. and 48.00 kg of sucrose was slowly added to the vessel. The vessel was covered and mixing continued until all the sucrose was dissolved.

When the sucrose was completely dissolved, the temperature controller was set to its lowest temperature and the solution was allowed to cool to 25° C. 0.02642 kg of chlorpheniramine maleate, 0.04954 kg of hydrocodone bitartrate, 0.2680 kg of artificial strawberry flavor powder, 0.2160 g of artificial bitter masking powder, and 0.0720 kg of sucralose were added to the syrup. Mixing was continued until all the solid was dissolved. 0.0720 kg of FD&C Red No. 40 aluminum lake and 0.1800 kg of titanium dioxide were then added through a #30 screen into the vessel with stirring continuing until a uniform dispersion was obtained. (Note: This example of the vehicle contains insoluble material which will settle when mixing is stopped.)

Final net weight of the mixture was determined, and, if necessary, purified water was added to compensate for evaporative loss.

### 6.11.4 Combining Coated Pellets and Vehicle Syrup for Formulation X

One method for combining the coated pellets and vehicle syrup to form a final Formulation X product is described below.

The vehicle syrup was stirred constantly such that a vortex is formed. After at least 10 minutes of such stirring, 24.00 g of coated beads was added to 130.8 g of vehicle to produce 24 doses or 120 mL of product.

### 6.12 Example 12

Example 12 describes a study conducted to evaluate effectiveness of Formulation X in humans. Specifically, Example 12 describes a study conducted to compare a single dose of extended release Formulation X to two doses of immediate release RLDs containing HC, PSE, or CPM used in combination in 16 healthy subjects. One objective of this study was to determine the bioequivalence of two formulations of Formulation X to the corresponding RLDs.

### 6.12.1 Absorption of Hydrocodone, Pseudoephedrine and Chlorpheniramine

### 6.12.1.1 Hydrocodone

Hydrocodone is well absorbed orally, but undergoes a significant first pass effect involving intestinal and hepatic metabolism. In previously published studies, following a single IR oral dose of 10 mg HC administered to 5 male human subjects, the mean peak serum concentration was 23.6±5.2 ng/mL, with a Tₘₐₓ of approximately 1.3±0.3 hours. “Hycodan®”, available at www.rxmed.com, accessed Jun. 23, 2008; Stout, P.; Farrell, L. Opioids—Effects on Human Performance and Behavior.” Forensic Science Review. 15(1): 29-59 (2003). All hydrocodone metabolites are active, and include hydromorphone, norcodeine, and 6-alpha and 6-beta hydroxy metabolites. Micromedex Health Care Series, DrugDex Evaluations “Hydrocodone Bitartrate/Ibuprofen,” available at http://www.thomsonhc.com/hcs, accessed Jul. 1, 2008, citing Cone, et al “Comparative metabolism of hydrocodone in man, rat, guinea pig, rabbit, and dog,” Drug Metab Divas 6:488-493 (1978).

Table 7 provides a table comparing parameters, such as AUC_{infinity}, Cₘₐₓ and T_{1/2}, relating to serum levels of hydrocodone (HC) obtained in patients upon administering one dose of “Formulation X” vs two doses of HC reference listed drug (RLD). Treatment A corresponds to one dose of “Formulation X comprising 15 mg HC, 120 mg PSE and 8 mg CPM. Treatment C corresponds to two doses of a cocktail of three single RLDs comprising 7.5 mg HC, 60 mg PSE and 4 mg CPM.

In this study (as shown in Table 7, Treatments A and C), following the administration of Formulation X containing 15 mg HC, 120 mg PSE, and 8 mg CPM to 16 human subjects, the mean peak serum concentration (Cₘₐₓ) of HC was 17.54-4.75 ng/mL, compared to a mean peak serum concentration of 25.64-6.56 ng/mL for the 2 doses of RLD containing 7.5 mg HC each administered at 0 and 6 hours. With Formulation X, a median Tₘₐₓ was 4 hours, compared to a median Tₘₐₓ of 7 hours for the 2 doses of RLD. The difference in Tₘₐₓ for Formulation X, as compared to what is seen in the scientific literature, is due to the fact that the extended release pellets in Formulation X release HC over a period of time to achieve a 12 hour dose. Because two doses of the RLD are administered in this study (as compared to one dose in the previously published studies), however, the peak serum concentration (Cₘₐₓ) achieved with the two doses of RLD is reached at a later time, as compared to that with Formulation X.

TABLE 7
Formulation X vs RLDs - Hydrocodone

| | | | | | Ratio of Geometric Means |
|---|---|---|---|---|---|
| | | | Median | | [Test/Reference |
| | | | Min., | Geometric | Treatments] |
| Parameter | Treatment | Mean (SD) | Max. | Mean^{a} | Point Estimate (90% CI)^{a} |
| AUC | A | 238.78 | 203.18 | 227.09 | [A/C] 0.9328 |
| (ng * hr/mL) | | (80.93) | 126.1, | | (0.8482, 1.0258) |
| | | | 393.9 | | |
| | B | 163.43 | 146.46 | 155.67 | [B/D] 0.9510 |
| | | (57.69) | 93.5, | | (0.8647, 1.0459) |
| | | | 323.7 | | |
| | C | 254.49 | 216.28 | 243.46 | — |
| | | (79.97) | 150.7, | | |
| | | | 399.9 | | |
| | D | 172.30 | 157.23 | 163.69 | — |
| | | (56.65) | 83.9, | | |
| | | | 285.1 | | |
| AUC_{LAST} | A | 207.40 | 183.10 | 199.11 | [A/C] 0.8559 |
| (ng * hr/mL) | | (62.44) | 112.7, | | (0.7780, 0.9416) |
| | | | 327.8 | | |
| | B | 136.22 | 128.47 | 132.53 | [B/D] 0.8495 |
| | | (34.05) | 82.2, | | (0.7722, 0.9345) |
| | | | 216.5 | | |
| | C | 241.83 | 209.61 | 232.64 | — |
| | | (70.46) | 144.2, | | |
| | | | 371.3 | | |
| | D | 163.23 | 149.28 | 156.01 | — |
| | | (50.19) | 81.5, | | |
| | | | 259.0 | | |
| C_{MAX} | A | 17.54 | 16.53 | 16.88 | [A/C] 0.6796 |
| (ng/mL) | | (4.75) | 8.1, 25.1 | | (0.6052, 0.7632) |
| | B | 10.68 | 10.45 | 10.51 | [B/D] 0.6472 |
| | | (1.97) | 7.6, 14.1 | | (0.5763, 0.7268) |
| | C | 25.64 | 25.02 | 24.84 | — |
| | | (6.56) | 13.7, 38.3 | | |
| | D | 16.70 | 16.52 | 16.24 | — |
| | | (3.74) | 7.8, 23.9 | | |
| T_{MAX} | A | — | 4.00 | — | — |
| (hours) | | | 1.0, 9.0 | | |
| | B | — | 6.00 | — | — |
| | | | 1.0, 7.0 | | |
| | C | — | 7.00 | — | — |
| | | | 1.0, 8.0 | | |
| | D | — | 7.00 | — | — |
| | | | 1.0, 9.0 | | |
| T_{1/2} | A | 7.22 | 6.79 | — | — |
| (hours) | | (1.45) | 4.7, 9.2 | | |
| | B | 7.62 | 6.93 | — | — |
| | | (2.34) | 5.0, 13.7 | | |
| | C | 4.38 | 4.19 | — | — |
| | | (0.83) | 3.2, 6.7 | | |
| | D | 4.53 | 4.44 | — | — |
| | | (0.86) | 3.1, 6.1 | | |

Treatment A: Test formulation #1 - Formulation X 1q: 15 mg HC, 120 mg PSE, 8 mg CPM
Treatment B: Test formulation #2 - Formulation X 1q: 10 mg HC, 120 mg PSE, 8 mg CPM
Treatment C: Reference formulation #1 (for Treatment A) - RLD 2q: 7.5 mg HC, 60 mg PSE, 4 mg CPM
Treatment D: Reference formulation #2 (for Treatment B) - RLD 2q: 5 mg HC, 60 mg PSE, 4 mg CPM
AUC = total area under the plasma concentration-time curve from 0 extrapolated to infinity;
AUC_{LAST} = area under the plasma concentration-time curve from 0 to the last quantifiable plasma concentration;
C_{MAX} = maximum observed plasma concentration;
CPM = chlorpheniramine maleate;
HC = hydrocodone bitartrate;
PSE = pseudoephedrine hydrochloride;
T_{MAX} = time of maximum plasma concentration,
T_{1/2} = elimination half-life
^{a}Exponentiated results of analysis of log-transformed values

### 6.12.1.2 Pseudoephedrine

Pseudoephedrine is readily and almost completely absorbed from the GI tract and there is no evidence of first-pass metabolism. In previously published studies, T_{in a}, was observed at 1.5-2.4 hours following administration and bioavailability is the same across formulations and is unaffected by food. Micromedex Health Care Series. DrugDex Evaluations “Pseudoephedrine,” available at http://www.thomsonhc.com/hcs, accessed Jul. 1, 2008; “Common cold and influenza management” MedScape available at www.medscape.com/viewarticle/466063_{—}3, accessed Jun. 26, 2008; Graves D A, et al. “Influence of a standard meal on the absorption of a controlled release pseudoephedrine suspension.” Biopharm Drug Dispos. May-June; 9(3):267-72 (1988).

Table 8 provides a table comparing parameters, such as AUC_{infinity}, Cₘₐₓ and T_{Y}, relating to serum levels of pseudoephedrine (PSE) obtained in patients upon administering one dose of “Formulation X” vs two doses of PSE RLD. Treatment A corresponds to one dose of “Formulation X comprising 15 mg FTC, 120 mg PSE and 8 mg CPM. Treatment C corresponds to two doses of a cocktail of three single RLDs comprising 60 mg PSE, 7.5 mg HC and 4 mg CPM.

In this study (as shown in Table 8, Treatments A and C), following the administration of Formulation X containing 15 mg HC, 120 mg PSE, and 8 mg CPM to 16 human subjects, the mean peak serum concentration (Cᵣ—) of PSE was 292.05±49.94 ng/mL, compared to 345.47±78.46 ng/mL after 2 doses of the IR RLD containing 60 mg PSE each. Median Tₘₐₓ was observed 5 hours following dosing of Formulation X, compared to 7 hours following dosing of the RLD. As explained above, the ER pellets in Formulation X release PSE over a period of time to achieve a 12 hour dose. As a result, the peak serum concentration of the RLD in this study is reached at a later time.

TABLE 8
Formulation X vs RLDs - Pseudoephedrine

| | | | | | Ratio of Geometric Means |
|---|---|---|---|---|---|
| | | | | | [Test/Reference |
| | | Mean | Median | Geometric | Treatments] |
| Parameter | Treatment | (SD) | Min., Max. | Mean^{a} | Point Estimate (90% CI)^{a} |
| AUC | A | 3919.80 | 4203.51 | 3844.66 | [A/C] 0.9545 |
| (ng * hr/mL) | | (769.92) | 2645.4, | | (0.8855, 1.0288) |
| | | | 4965.8 | | |
| | B | 4042.92 | 4110.85 | 3944.76 | [B/D] 0.9880 |
| | | (969.30) | 2496.4, | | (0.9166, 1.0649) |
| | | | 6879.8 | | |
| | C | 4149.31 | 3970.85 | 4028.02 | — |
| | | (1034.06) | 2608.5, | | |
| | | | 6211.7 | | |
| | D | 4107.13 | 4151.74 | 3992.82 | — |
| | | (1013.50) | 2682.8, | | |
| | | | 6316.5 | | |
| AUC_{LAST} | A | 3291.46 | 3558.28 | 3201.77 | [A/C] 0.9551 |
| (ng * hr/mL) | | (764.26) | 1963.7, | | (0.8676, 1.0514) |
| | | | 4128.8 | | |
| | B | 3237.90 | 3175.08 | 3111.09 | [B/D] 0.9128 |
| | | (950.08) | 1432.8, | | (0.8292, 1.0049) |
| | | | 5868.5 | | |
| | C | 3519.47 | 3525.79 | 3352.38 | — |
| | | (1087.08) | 1735.2, | | |
| | | | 5738.7 | | |
| | D | 3535.29 | 3607.51 | 3408.26 | — |
| | | (979.99) | 1983.6, | | |
| | | | 5543.1 | | |
| C_{MAX} | A | 292.05 | 288.86 | 287.79 | [A/C] 0.8526 |
| (ng/mL) | | (49.94) | 196.3, 366.2 | | (0.7729, 0.9406) |
| | B | 275.35 | 262.75 | 269.39 | [B/D] 0.7900 |
| | | (60.23) | 171.8, 398.4 | | (0.7161, 0.8715) |
| | C | 345.47 | 331.05 | 337.53 | — |
| | | (78.46) | 216.1, 531.8 | | |
| | D | 347.95 | 354.76 | 341.00 | — |
| | | (69.91) | 219.0, 446.2 | | |
| T_{MAX} | A | — | 5.00 | — | — |
| (hours) | | | 3.0, 10.0 | | |
| | B | — | 5.00 | — | — |
| | | | 3.0, 10.0 | | |
| | C | — | 7.00 | — | — |
| | | | 6.5, 8.0 | | |
| | D | — | 8.00 | — | — |
| | | | 6.5, 9.0 | | |
| T_{1/2} | A | 6.48 | 6.52 | — | — |
| (hours) | | (1.40) | 3.5, 8.4 | | |
| | B | 7.28 | 7.27 | — | — |
| | | (1.73) | 4.5, 11.1 | | |
| | C | 5.06 | 4.96 | — | — |
| | | (0.90) | 3.6, 6.7 | | |
| | D | 4.93 | 4.95 | — | — |
| | | (0.96) | 3.5, 6.8 | | |

Treatment A: Test formulation #1 - Formulation X 1q: 15 mg HC, 120 mg PSE, 8 mg CPM
Treatment B: Test formulation #2 - Formulation X 1q: 10 mg HC, 120 mg PSE, 8 mg CPM
Treatment C: Reference formulation #1 (for Treatment A) - RLD 2q: 7.5 mg HC, 60 mg PSE, 4 mg CPM
Treatment D: Reference formulation #2 (for Treatment B) - RLD 2q: 5 mg HC, 60 mg PSE, 4 mg CPM
AUC = total area under the plasma concentration-time curve from 0 extrapolated to infinity;
AUC_{LAST} = area under the plasma concentration-time curve from 0 to the last quantifiable plasma concentration;
C_{MAX} = maximum observed plasma concentration;
CPM = chlorpheniramine maleate;
HC = hydrocodone bitartrate;
PSE = pseudoephedrine hydrochloride;
T_{MAX} = time of maximum plasma concentration,
T_{1/2} = elimination half-life
^{a}Exponentiated results of analysis of log-transformed values

### 6.12.1.3 Chlorpheniramine

Chlorpheniramine is rapidly and completely absorbed following oral administration. In previously published studies, the drug appeared in the systemic circulation within 30 to 60 minutes and reached Cₘₐₓ in 2 hours, with the concentration decreasing over the next 46 hours. Peets, E et al. “Metabolism of chlorpheniramine maleate in man,” J. Pharmacol. Exp. Ther. 180:464-474 (1972); “Micromedex Health Care Series. DrugDex Evaluations “Chlorpheniramine,” available at http://www.thomsonhc.com/hcs, accessed Jul. 1, 2008. CPM has a 41±16% oral bioavailability, and its absorption, but not its bioavailability, is delayed by food intake. Rumore, M. M, pharmacokinetics of chlorpheniramine,” Drug Intel. Clin. Pharm. 18:701-707 (1984). However, CPM appears to undergo substantial metabolism in the GI mucosa during absorption and on first pass through the liver. Limited data indicate that about 25 to 45% of a single oral dose of CPM as a conventional tablet, and 35 to 60% as a solution reaches the systemic circulation as unchanged drug. Limited data also indicate that the bioavailability of extended-release preparations of the drug may be reduced compared with that of conventional tablets or oral solution. Micromedex Health Care Series: Chlorpheniramine, ibid; Therapeutic Goods Administration (Australia) “Core sedating antihistamines product information” available at www.tga.gov.au/npmeds/pi-sedatingantihistamine.rtf, accessed Jun. 26, 2008.

Table 9 provides a table comparing parameters, such as AUC_{infinity}, Cₘₐₓ and T_{1/2}, relating to serum levels of chlorpheniramine (CPM) obtained in patients upon administering one dose of “Formulation X” vs two doses of CPM RLD. Treatment A corresponds to one dose of “Formulation X comprising 15 mg HC, 120 mg PSE and 8 mg CPM. Treatment C corresponds to two doses of a cocktail of three single RLDs comprising 4 mg CPM, 7.5 mg HC and 60 mg PSE.

In this study (as shown in Table 9, Treatments A and C), following the administration of Formulation X containing 15 mg HC, 120 mg PSE, and 8 mg CPM to 16 human subjects, the mean peak serum concentration (Cₘₐₓ) of CPM was 21.20±6.30 ng/mL compared to 28.89±7.92 ng/mL after 2 doses of the RLD containing 4 mg

TABLE 9
Formulation X vs RLDs - Chlorpheniramine

| | | | | | Ratio of Geometric Means |
|---|---|---|---|---|---|
| | | | | | [Test/Reference |
| | | Mean | Median | Geometric | Treatments] |
| Parameter | Treatment | (SD) | Min., Max. | Mean^{a} | Point Estimate (90% CI)^{a} |
| AUC | A | 1217.29 | 942.38 | 881.45 | [A/C] 1.2924 |
| (ng * hr/mL) | (N = 15) | (943.95) | 341.3, | | (0.9733, 1.7160) |
| | | | 3388.7 | | |
| | B | 770.03 | 610.52 | 694.03 | [B/D] 1.0339 |
| | (N = 14) | (429.72) | 347.4, | | (0.7786, 1.3728) |
| | | | 1681.5 | | |
| | C | 910.18 | 704.63 | 682.05 | — |
| | | (600.61) | 398.6, | | |
| | | | 2659.5 | | |
| | D | 964.21 | 732.82 | 671.29 | — |
| | | (756.40) | 347.8, | | |
| | | | 3316.7 | | |
| AUC_{LAST} | A | 355.69 | 359.71 | 340.95 | [A/C] 0.8457 |
| (ng * hr/mL) | | (112.69) | 228.5, 673.7 | | (0.7564, 0.9454) |
| | B | 329.90 | 293.92 | 314.81 | [B/D] 0.7525 |
| | | (110.92) | 174.2, 576.1 | | (0.6731, 0.8413) |
| | C | 421.31 | 417.25 | 403.17 | — |
| | | (134.24) | 223.2, 753.5 | | |
| | D | 444.78 | 398.39 | 418.32 | — |
| | | (169.87) | 241.3, 914.1 | | |
| C_{MAX} | A | 21.20 | 19.65 | 20.37 | [A/C] 0.7309 |
| (ng/mL) | | (6.30) | 13.8, 33.8 | | (0.6440, 0.8295) |
| | B | 19.18 | 18.57 | 18.35 | [B/D] 0.6395 |
| | | (6.15) | 10.1, 34.8 | | (0.5635, 0.7258) |
| | C | 28.89 | 29.21 | 27.88 | — |
| | | (7.92) | 14.9, 45.4 | | |
| | D | 30.84 | 27.66 | 28.69 | — |
| | | (13.51) | 16.8, 71.4 | | |
| T_{MAX} | A | — | 9.00 | — | — |
| (hours) | | | 4.0, 24.0 | | |
| | B | — | 10.00 | — | — |
| | | | 5.0, 24.0 | | |
| | C | — | 9.50 | — | — |
| | | | 8.0, 12.0 | | |
| | D | — | 9.00 | — | — |
| | | | 8.0, 12.0 | | |
| T_{1/2} | A | 41.71 | 27.50 | — | — |
| (hours) | (N = 15) | (43.33) | 11.3, 181.2 | | |
| | B | 27.50 | 19.84 | — | — |
| | (N = 14) | (18.13) | 12.0, 76.1 | | |
| | C | 19.37 | 17.96 | — | — |
| | | (9.66) | 6.8, 39.0 | | |
| | D | 20.30 | 14.22 | — | — |
| | | (21.08) | 6.2, 96.0 | | |

Treatment A: Test formulation #1 - Formulation X 1q: 15 mg HC, 120 mg PSE, 8 mg CPM
Treatment B: Test formulation #2 - Formulation X 1q: 10 mg HC, 120 mg PSE, 8 mg CPM
Treatment C: Reference formulation #1 (for Treatment A) - RLD 2q: 7.5 mg HC, 60 mg PSE, 4 mg CPM
Treatment D: Reference formulation #2 (for Treatment B) - RLD 2q: 5 mg HC, 60 mg PSE, 4 mg CPM
AUC = total area under the plasma concentration-time curve from 0 extrapolated to infinity;
AUC_{LAST} = area under the plasma concentration-time curve from 0 to the last quantifiable plasma concentration;
C_{MAX} = maximum observed plasma concentration;
CPM = chlorpheniramine maleate;
HC = hydrocodone bitartrate;
PSE = pseudoephedrine hydrochloride;
T_{MAX} = time of maximum plasma concentration,
T_{1/2} = elimination half-life
^{a}Exponentiated results of analysis of log-transformed values CPM each. The median Tₘₐₓ was achieved at 9 hours following dosing of Formulation X and 9.50 hours following the RLD.

### 6.12.2 Elimination and Excretion of Hydrocodone, Pseudoephedrine and Chlorpheniramine

### 6.12.2.1 Hydrocodone

The elimination half-life of the parent-compound of hydrocodone is between 3.8-4.5 hours. Micromedex Health Care Series: Hydrocodone, ibid. About 70% of total excretion occurring in the first 24 hours after a single oral dose. Id.; Stout, P.; Farrell, L. “Opioids Effects on Human Performance and Behavior.” Forensic Science Review, 15(1): 29-59. (2003).

In this study (as shown in Table 7, Treatments A and C), the T_{1/2} of Formulation X for HC was measured to be 7.22±1.45 hours, compared to 4.38±0.83 hours for the RLD. This difference was expected because Formulation X is an extended release drug.

### 6.12.2.2 Pseudoephedrine

PSE and its metabolite are excreted in the urine, with up to 90% of a dose being excreted unchanged within 24 hours of dosing. PSE has a half-life of approximately 9-16 hours, which can be affected by urinary pH, prolonging it when alkaline (pH 8) and reducing it when acidic (pH 5). Wishart, D., et al. “DrugBank: a comprehensive resource for in silico drug discovery and exploration,” Nucleic Acids Res. 34: D668-72 (2006); Micromedex Health Care Series: Pseudoephedrine, ibid.

In this study (as shown in Table 8, Treatments A and C), the T_{1/2} of Formulation X for PSE was measured to be 6.48±1.40 hours, compared to 5.06±0.90 hours for the RLD. This difference was expected because Formulation X is an extended release drug.

### 6.12.2.3 Chlorpheniramine

Elimination from the body of chlorpheniramine is primarily by metabolism to monodesmethyl and didesmethyl compounds with up to 26% excreted in the urine. Renal elimination accounts for approximately 50% total excretion with 3%-18% as unchanged drug. Renal excretion increases with increased urine flow and lower pH. See Micromedex Health Care Series: Chlorpheniramine, ibid. Less than 1% is excreted in the feces. The half-life for CPM is 20±5 hours with a measured clearance of 1.7±0.1 mL/min/kg. Rumore, ibid.

In this study (as shown in Table 9, Treatments A and C), the T_{1/2} of Formulation X for CPM was measured to be 41.71±43.33 hours, compared to 19.37±9.66 hours for the RLD. However, the sampling protocol only measured CPM for 24 hours following the administration of Formulation X. Measurement over only 24 hours was probably insufficient to accurately measure the elimination half-life of CPM in Formulation X, and may have contributed to the variability seen in T_{1/2}. It is expected that bioequivalence will be observed.

The data shown in Tables 2-4 do not necessarily reflect the previously published values discussed above regarding and T₁₁₂. The reason for this is that Formulation X is an extended release formulation. The published values for T_{1/2} and Tₘₐₓ are for drug that is immediately available for uptake into the blood and subsequent removal. Formulation X does not release all of the drugs immediately, so Tₘₐₓ is delayed and T_{1/2} and Tₘₐₓ may be shifted because drug is constantly being added for several hours after dosing. Likewise, the RLD values reported in Tables 2-4 do not necessarily reflect previously published values because values in the Tables are for two doses given 6 hours apart instead of the one dose that was used to determine values in the previously published studies. The second dose of the RLDs in the current study causes a second spike in drug concentration. As a result, absolute peak concentration of drug is achieved after the second dosing of the RLD. As a result, the Tₘₐₓ for the RLDs in Tables 2-4 appear delayed, but this is due only to the dosing protocol.

### 6.12.3 Bioequivalence of Hydrocodone, Pseudoephedrine and Chlorpheniramine

For a single dose study, the FDA considers AUC to be the only relevant PK parameter for showing BE; however, the FDA does request information on other PK parameters. Tables 2-4 contain the PK data for two different formulations of Formulation X and their respective RLDs. Table 2 shows that for HC, comparing Treatment A (Formulation X comprising 15 mg HC) and Treatment C(RLD comprising 7.5 mg HC, administered two times), the point estimate of AUC for Formulation X was 93.28% of the RLD with a 90% confidence interval (CI) of 84.82% to 102.58%. This meets the FDA's BE standard of 80-125% at 90% CI, and therefore HC achieved bioequivalence in this study. Tables 3 shows that for PSE, comparing Treatment A and Treatment C, the point estimate of AUC for Formulation X was 95.45% of the RLD with a 90% CI of 88.55% to 102.88%, achieving bioequivalence. Tables 4 shows that for CPM, comparing Treatment A and Treatment C, the point estimate of AUC for Formulation X was 129.24% of the RLD with a 90% CI of 97.33% to 171.60%. This value does not established BE. It is expected, however, that the failure to establish BE for CPM in this study was not due to a failure of Formulation X to achieve BE for CPM (in addition to HC and PSE), but instead due to a failure of the current study to account for the long half life of CPM during serum collection. Specifically, when final serum samples were collected, the CPM concentration was not yet decaying to the point that an accurate T_{1/2} could be determined. As a result, the extrapolation required to determine AUC was unreliable. This was true for the CMP RLDs and Formulation X. Future trials will establish that when an appropriate sampling time is used, the CPM data will show that Formulation X is BE to the CPM RLD.

In sum, the 90% confidence limits of AUC for Formulation X fell within 80%-125% of the RLDs for HC and PSE, indicating bioequivalence of at least these two drugs. In this particular study, the AUC for CPM did not definitively establish bioequivalence, as the 90% confidence limits for Formulation X, as compared to the CPM RLD, fell between 97.33 to 171.60%. Analysis of CPM AUC was complicated by high intrasubject variability and large differences between AUC_{0-INF} and AUC_{0-LAST} observed for all formulations. A larger study sample to address variability and longer sampling times to account for the long elimination half-life of CPM (20-24 hours by one source) will establish CPM bioequivalence of Formulation X to the CPM RLD. “Drug information: chlorpheniramine,” available at: www.accessmedicine.com/drugs.aspx?index=C, accessed May 15, 2007.

An analytical method is employed to determine the amount or ratios of APIs that should be used when preparing a formulation comprising an antihistamine, an antitussive and a decongestant as APIs, so that the formulation exhibits extended release (ER) release of all three APIs when administered to a patient.

While the invention has been described and exemplified in sufficient detail for those skilled in this art to make and use it, various alternatives, modifications, and improvements should be apparent without departing from the spirit and scope of the invention. The examples provided herein are representative of certain specific embodiments, are exemplary, and are not intended as limitations on the scope of the invention. Modifications therein and other uses will occur to those skilled in the art. These modifications are encompassed within the spirit of the invention and are defined by the scope of the claims.

It will be readily apparent to a person skilled in the art that varying substitutions and modifications may be made to the invention disclosed herein without departing from the scope and spirit of the invention. As such, the present invention is not to be limited in scope by the specific embodiments disclosed in the examples which are intended as illustrations of a few aspects of the invention and any embodiments that are functionally equivalent are within the scope of this invention. Indeed, various modifications of the invention in addition to those shown and described herein will become apparent to those skilled in the art and are intended to fall within the scope of the appended claims.

All patents and publications mentioned in the specification are indicative of the levels of those of ordinary skill in the art to which the invention pertains. All patents and publications are herein incorporated by reference to the same extent as if each individual publication was specifically and individually indicated to be incorporated by reference.

### 6.13 Example 13

Example 13 describes the results of the in vitro studies conducted to evaluate the release profile of pseudoephedrine, hydrocodone and chlorpheniramine in Formulation X in comparison to release profile of each of these drugs from coated beads comprising chlorpheniramine, hydrocodone and pseudoephedrine in a single bead.

Formulation X was prepared as described in Example 11. Coated beads comprising chlorpheniramine, hydrocodone and pseudoephedrine in a single bead were prepared in accordance with section 6.11.1 and 6.11.2 of Example 11. The time zero (t=0) sample was analyzed within 1 week from when production was completed; other samples were stored at 25° C. at a chamber with 60% humidity ( 25/60) and analyzed in 1 month (1 mo), 3 months (3 mo), 6 months (6 mo), 9 months (9 mo) and 12 months (12 mo) from when production was completed, respectively. Assay determinations were made by HPLC. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 5 mL samples at the following time points: 0.5, 4, 8, and 12 hours.

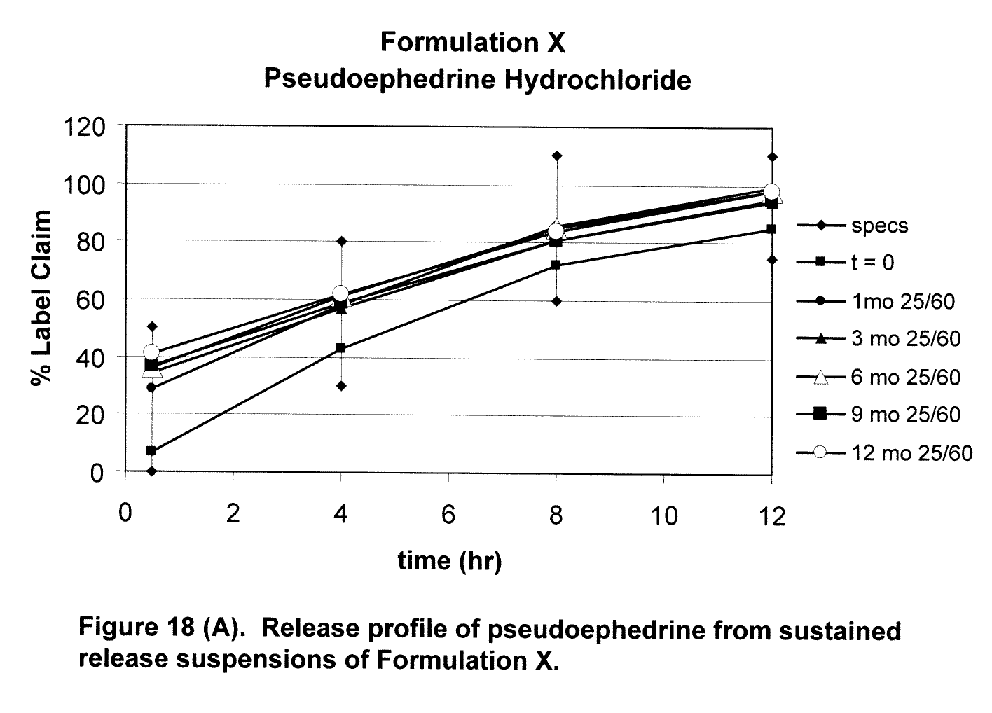

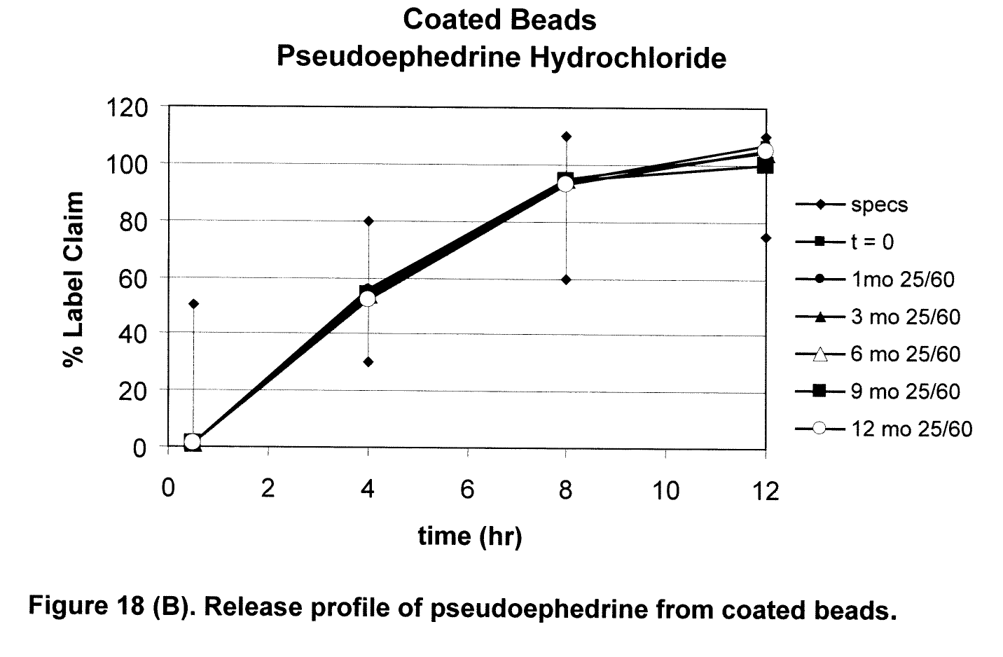

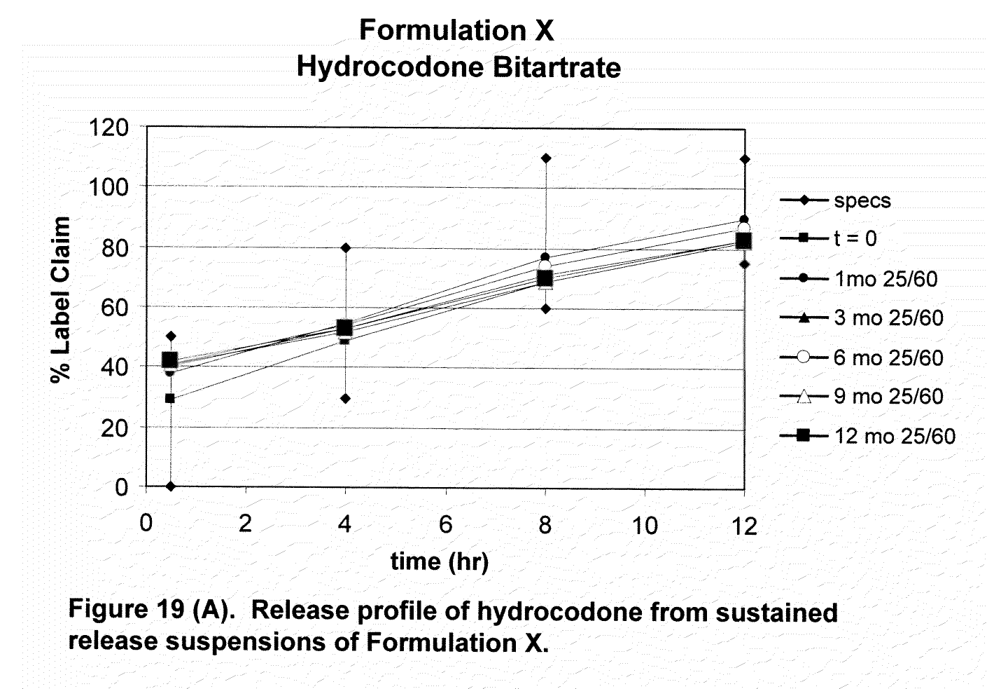

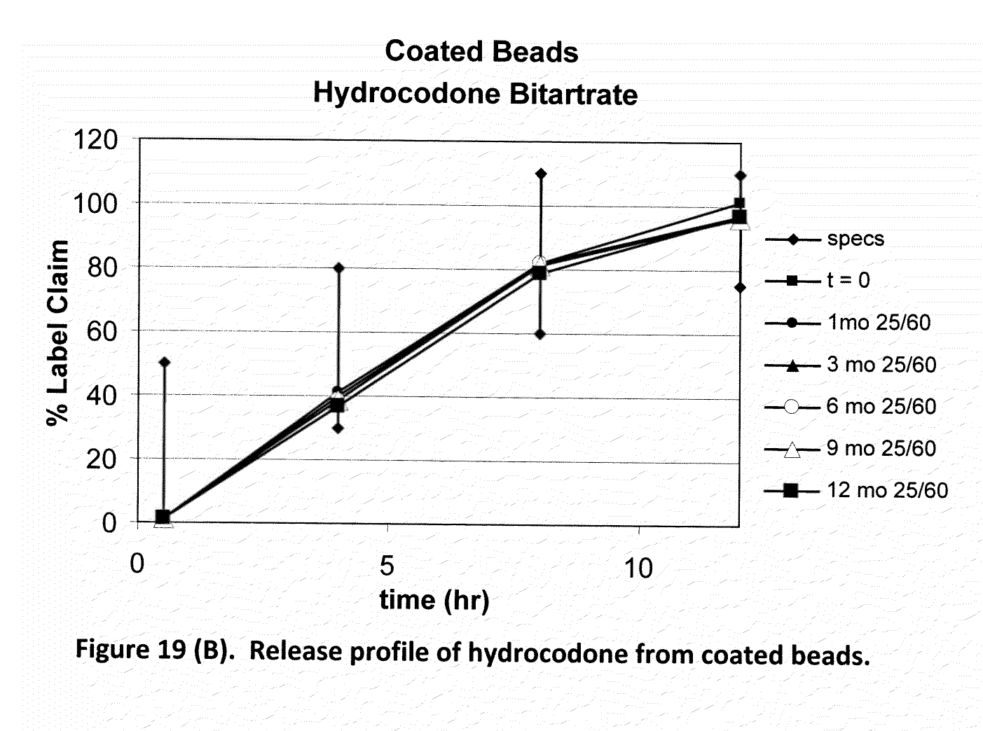

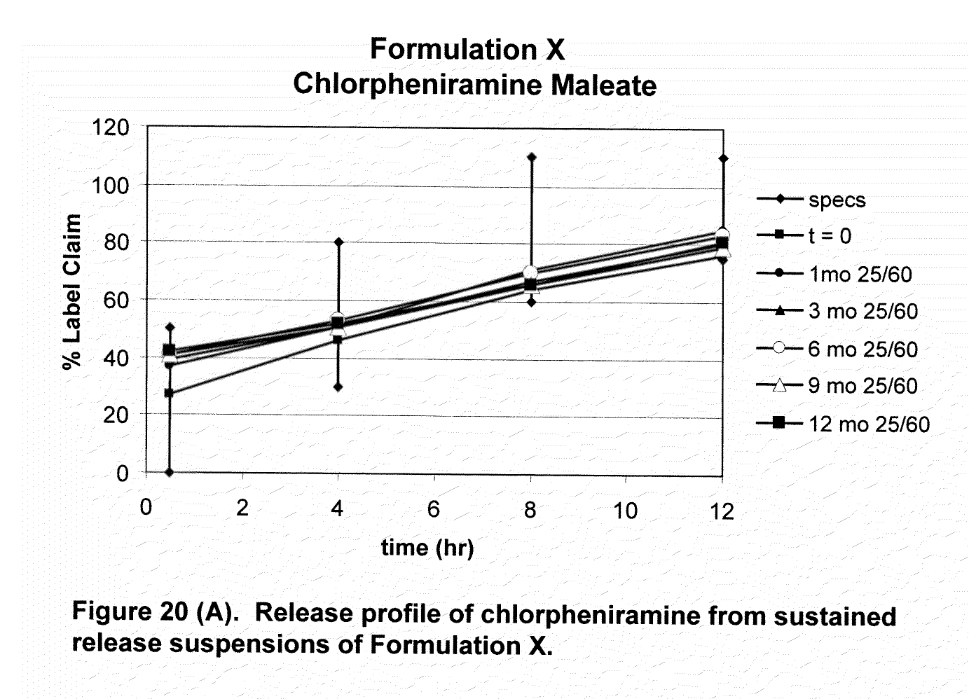

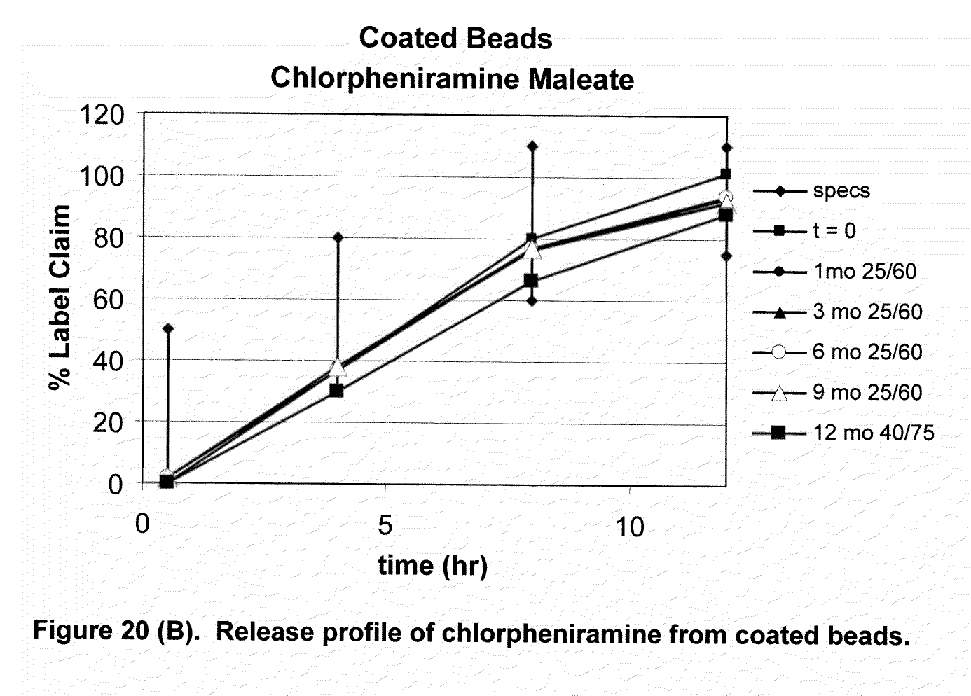

FIG. 18(A) shows the release profile of pseudoephedrine from the liquid form sustained release suspension of Formulation X. FIG. 18(B) shows the release profile of pseudoephedrine from the coated beads. FIG. 19(A) shows the release profile of hydrocodone from the liquid form sustained release suspension of Formulation X. FIG. 19(B) shows the release profile of hydrocodone from the coated beads. FIG. 20(A) shows release profile of chlorpheniramine from the liquid form sustained release suspension of Formulation X. FIG. 20(B) shows the release profile of chlorpheniramine from the coated beads.

FIGS. 18-20 demonstrate that the rate of release of the extended release portion of pseudoephedrine, hydrocodone and chlorpheniramine in Formulation X is comparable to the rate of release of pseudoephedrine, hydrocodone and chlorpheniramine from coated beads when the results are normalized for the presence of an immediate release portion of each of these drugs in Formulation X. The results of the experiment presented in FIGS. 18-20 show that the presence of ionic components, i.e., free drugs in their respective salt forms, in an immediate release phase does not interfere with an adequate rate of sustained release of each of these drugs from the extended release portion of Formulation X. FIGS. 18-20 also demonstrate that the rate of release of the extended release portion of pseudoephedrine, hydrocodone and chlorpheniramine in Formulation X tested at time zero is comparable with the rate of release of the extended release portion of pseudoephedrine, hydrocodone and chlorpheniramine in Formulation X stored for 1 month, 3 months, 6 months, 9 months and 12 months post production. Thus, the results of the experiment presented in FIGS. 18-20 also show that Formulation X maintains stability and an adequate rate of sustained release of each of these drugs after storage of Formulation X at room temperature conditions for 1 month, 3 months, 6 months, 9 months and 12 months.

### 6.14 Example 14

Example 14 describes the results of in vitro studies conducted to compare the release profile of a liquid form controlled release composition comprising base forms of drugs in the dispersed phase with the release profile of a liquid form controlled release composition comprising salt forms of drugs in the dispersed phase.

The experiment was carried out with two types of compositions, each designed to deliver the equivalent of 30 mg of pseudoephedrine hydrochloride (7.5 mg IR and 22.5 mg ER), 7.5 mg of hydrocodone bitartrate (1.87 IR and 5.63 mg ER), and 6 mg of chlorpheniramine maleate (1.5 mg IR and 4.5 mg ER) in 5 ml of the liquid dosage form. The first type contained 18.4 mg of pseudoephedrine, 3.41 mg of hydrocodone and 1.58 mg of chlorpheniramine, wherein the drugs were used in their base forms, bound to the alginic acid resin. The drug-loaded alginate beads were prepared, coated and dispersed into a dispersion medium containing 65% W/W of sucrose essentially as described in Example 10. The second type contained 22.5 mg of pseudophedrine hydrochloride, 5.62 mg of hydrocodone bitartrate and 4.5 mg of chlorpheniramine maleate, bound to sodium alginate. In the second type composition, the core beads were manufactured and coated essentially as described in sections 6.11.1 and 6.11.2, and such coated beads were dispersed into a dispersion medium containing 65% W/W of sucrose. In both cases, the dispersion medium contained the portion of each dose of the salt forms of the drugs designed for immediate release.

The samples were stored at room temperature for 3 weeks prior to analysis. Then, assay determinations were made by HPLC. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 1 mL samples at the following time points: 5, 30, 60, 90 minutes; 4, 8, 12, 16, 20, 24 hours.

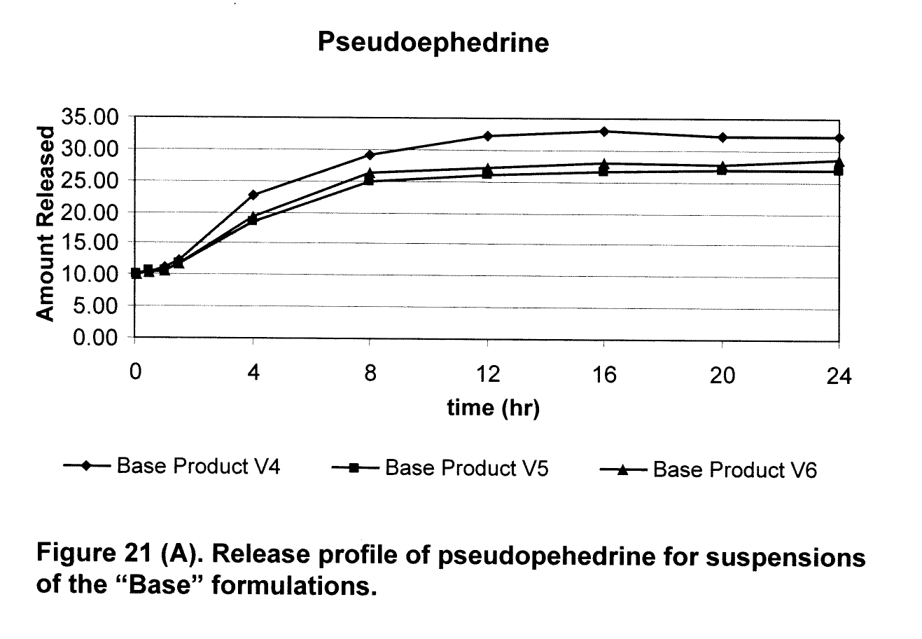

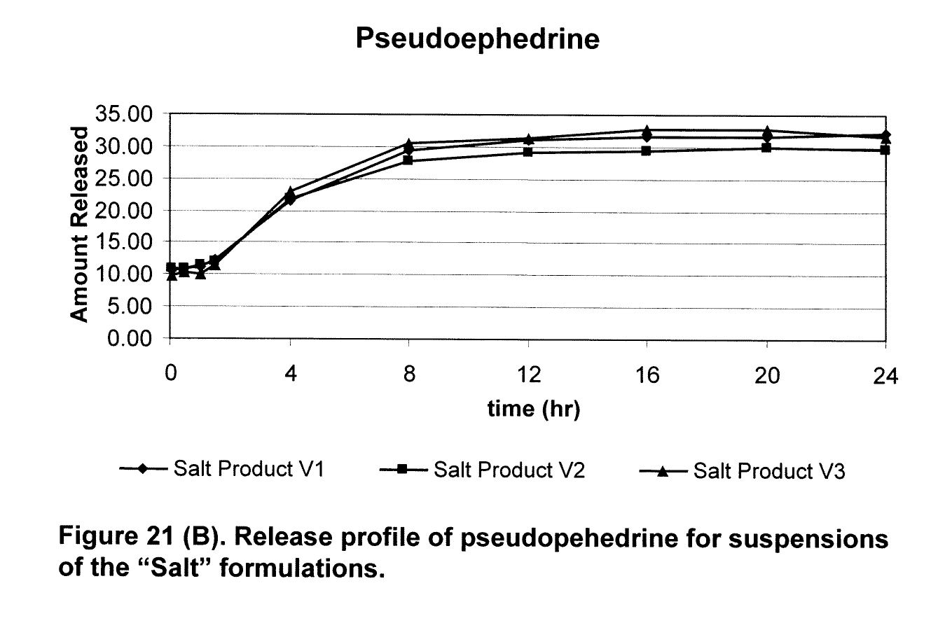

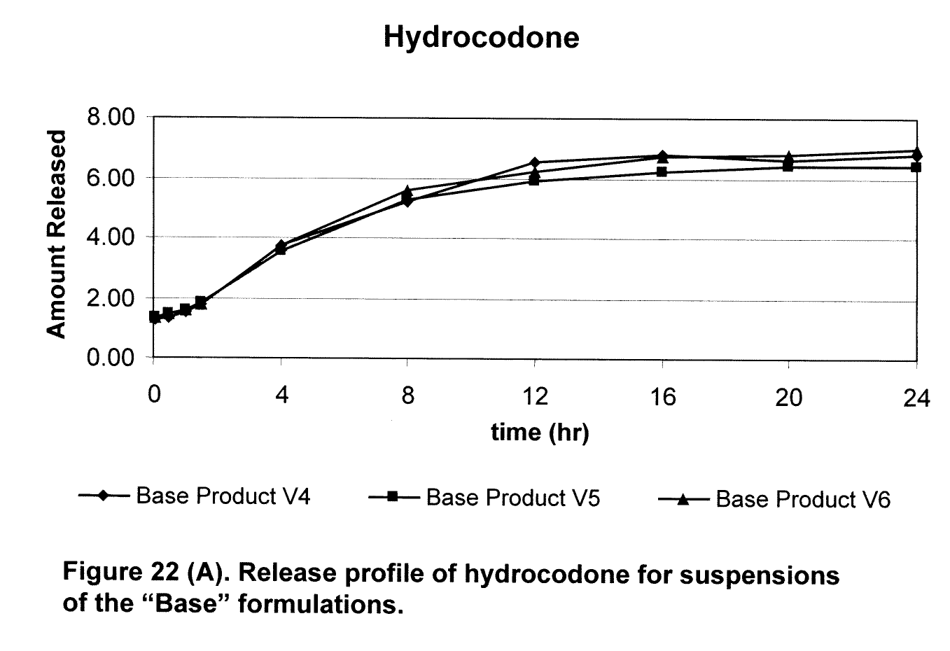

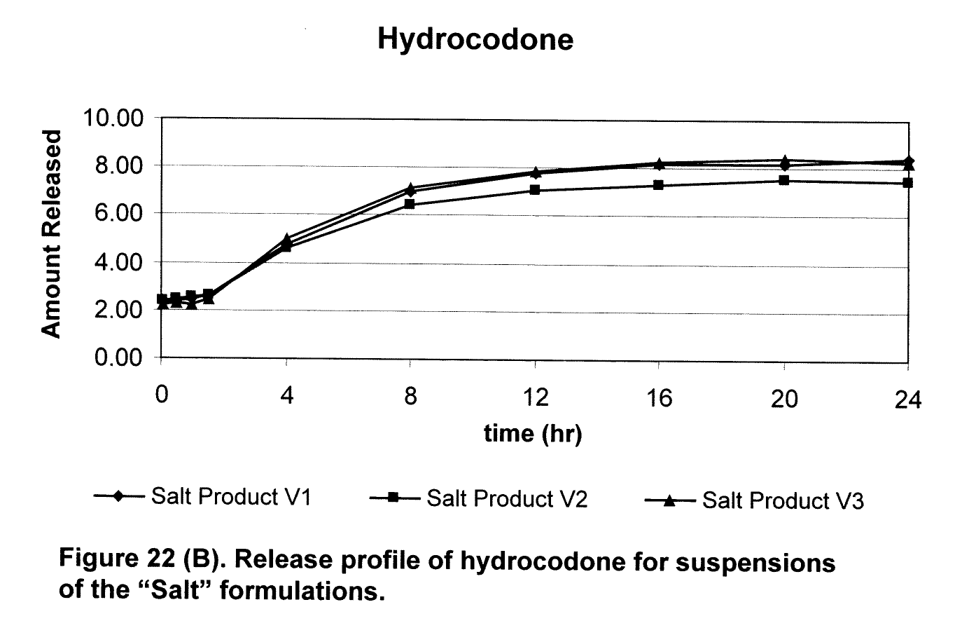

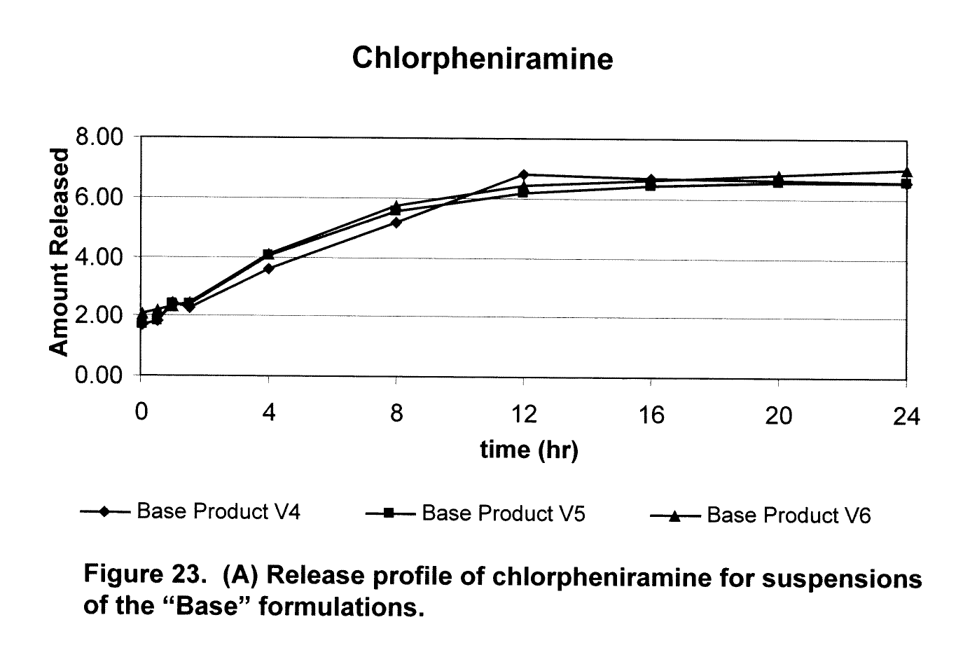

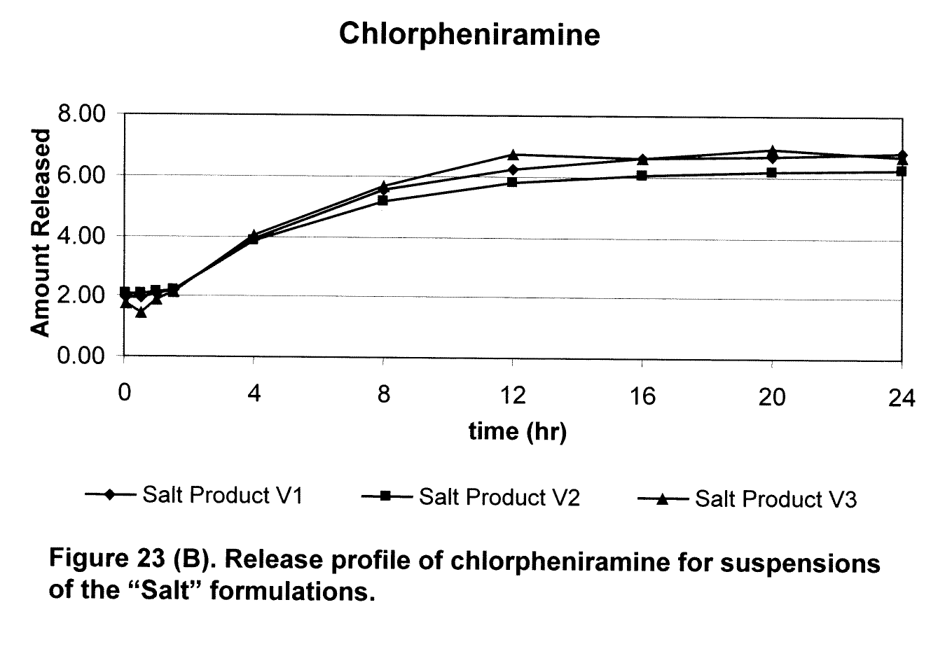

FIG. 21(A) shows the release profile of base formulations of pseudoephedrine. FIG. 21(B) shows the release profile of salt formulations of pseudoephedrine. FIG. 22(A) shows the release profile of base formulations of hydrocodone. FIG. 22(B) shows the release profile of salt formulations of hydrocodone. FIG. 23(A) shows the release profile of base formulations of chlorpheniramine. FIG. 23(B) shows the release profile of salt formulations of chlorpheniramine.

FIGS. 21-23 show that, in a liquid form sustained release compositions, the rate of release of salt forms of pseudoephedrine, hydrocodone and chlorpheniramine is comparable to the rate of release of base forms of pseudoephedrine, hydrocodone and chlorpheniramine, respectively. Specifically, these drugs in salt and base forms show about the same or not significantly different release characteristics at each time point of the experiment. The results of the experiment presented in FIGS. 21-23 show that salt forms of drugs may be used in liquid form controlled release compositions of the invention to achieve adequate sustained release profile of such drugs.

### 6.15 Example 15

Example 15 shows the release profile of hydrocodone from a liquid form sustained release composition comprising only one active ingredient, hydrocodone, in the dispersed phase.

The experiment was carried out using a liquid form controlled release composition comprising 10 mg of hydrocodone base bound to alginic acid matrix. The hydrocodone alginate beads were prepared to contain 10 mg hydrocodone bound to alginic acid and 20% lactose monohydrate. Table 10 outlines quantitative composition of the Hydrocodone Alginate Bead formulation.

TABLE 10
Hydrocodone Alginate Bead Formulation

| Component | Weight | |
|---|---|---|
| Batch Size | 250 | g |
| Hydrocodone base | 5 | g |
| Alginic acid | 5 | g |
| Lactose (20%) | 50 | g |
| Avicel PH101 | 190 | g |

Hydrocodone Alginate Beads were then coated with 1.5% Opadry/31.5% Eudragit RS30D (on a weight by weight basis). The coated hydrocodone alginate beads were dispersed in 5 mL of Syrup per 10 mg of hydrocodone. Table 11 outlines quantitative composition of the liquid dispersion of extended release pellets containing hydrocodone.

TABLE 11
Quantitative Composition of the Hydrocodone formulation:
Liquid Dispersion of Extended Release Pellets in Syrup

| Component | Percent Weight |
|---|---|
| Hydrocodone | 0.1376% |
| Alginic Acid | 0.1376% |
| Lactose Monohydrate | 1.376% |
| Avicel PH101 | 5.230% |
| Opadry Clear | 0.1541% |
| Eudragit RS30D | 2.915% |
| Triethylcitrate | 0.3241% |
| Sucrose | 58.14% |
| Purified Water | 31.58% |

The samples were assayed immediately after production was completed. Assay determinations were made by HPLC. Release testing was done using USP Apparatus 2 with paddles operating at 100 RPM. Initially, 750 mL of pH 1.2 buffer was placed in each vessel. After the 1.5 hour time point, 250 mL of pH adjusting solution was added to produce a pH of 6.8 and a volume of 1000 mL. An autosampler was programmed to withdraw approximately 1 mL samples at the following time points: 30 minutes, 1 hour, 1.5 hours, 2 hours, 3 hours, 4 hours, 6 hours, 8 hours and 10 hours.

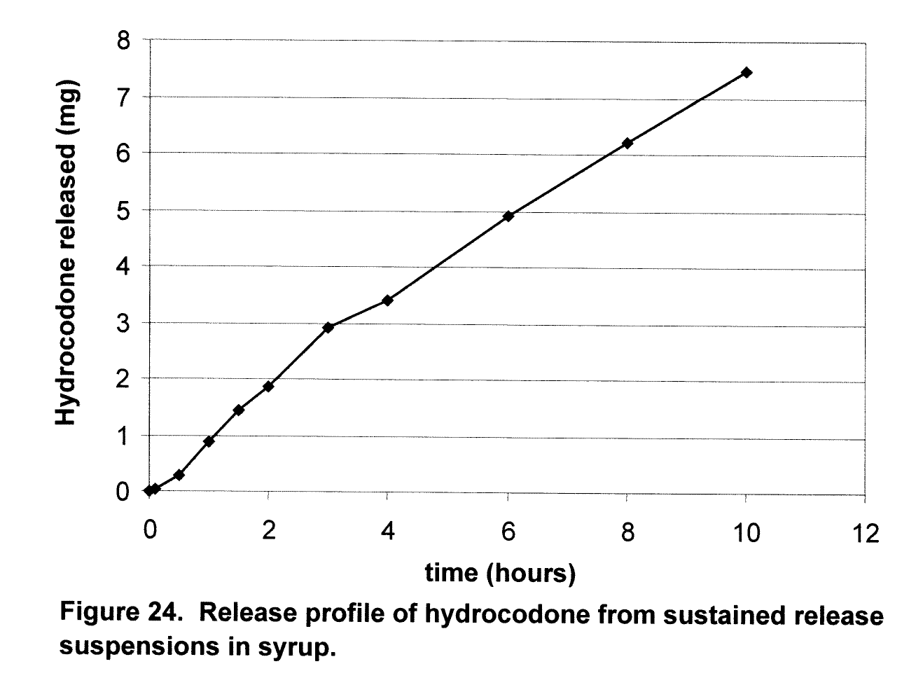

FIG. 24 shows release profile of hydrocodone from a liquid form controlled release composition comprising hydrocodone bound to alginic acid matrix.

FIG. 24 shows the rate of release of hydrocodone from a liquid form sustained release composition comprising beads containing only one active ingredient, hydrocodone, bound to an ion-exchange matrix and dispersed in Syrup.

What is claimed is:

1. A liquid form controlled release drug composition, comprising:
(a) a dispersed phase comprising particulates, pellets or beads comprising two or more drugs in a single particulate, pellet or bead;
(b) a diffusion-controlling membrane coating; and
(c) a dispersion medium.

2. The composition of claim 1, wherein the particulate, pellet or bead further comprises an ion-exchange matrix drug complex comprising a pharmaceutically acceptable ion-exchange matrix, wherein each of said two or more drugs associate with the ion-exchange matrix, and wherein the surface charge of the ion-exchange matrix is opposite that of said two or more drugs.

3. A liquid form controlled release drug composition, comprising:
(a) a dispersed phase comprising an ion-exchange matrix drug complex comprising a pharmaceutically acceptable ion-exchange matrix and a drug associated with the ion-exchange matrix, wherein the surface charge of the ion-exchange matrix is opposite that of the drug;
(b) a diffusion-controlling membrane coating; and
(c) a dispersion medium, wherein the dispersion medium comprises one or more drugs, wherein the dispersion medium does not comprise an ion-exchange matrix with the surface charge opposite that of at least one drug of said one or more drugs.

4. The composition of claim 2 or 3, wherein the ion-exchange matrix is sodium alginate.

5. The composition of claim 1 or 3, wherein at least one of the drugs is in a pharmaceutically acceptable salt form.

6. The composition of claim 1, wherein the dispersion medium comprises one or more drugs.

7. The composition of claim 6, wherein the dispersion medium does not comprise an ion-exchange matrix with the surface charge opposite that of at least one drug of said one or more drugs.

8. The composition of claim 6, wherein at least one drug of said one or more drugs in the dispersion medium is in an immediate release form.

9. The composition of claim 8, wherein at least one of the drugs in the dispersion medium is in a pharmaceutically acceptable salt form.

10. The composition of claim 1 or 3, which has a shelf life of 6 months or more.

11. The composition of claim 1 or 3, which maintains stability prior to administration to a patient for 6 months or more.

12. The composition of claim 1 or 3, wherein the diffusion-controlling membrane is selected from the group consisting of ethylcellulose, methylmethacrylate, cellulose esters, cellulose diesters, cellulose triesters, cellulose ethers, cellulose ester-ether, cellulose acylate, cellulose diacylate, cellulose triacylate, cellulose acetate, cellulose diacetate, cellulose triacetate, cellulose acetate propionate, cellulose acetate butyrate, and combinations thereof.

13. The composition of claim 11, wherein the diffusion-controlling membrane is ethylcellulose, methylmethacrylate, or combinations thereof.

14. The composition of claim 2 or 3, wherein the diffusion-controlling membrane coating is from about 20% to about 30% by weight based on the total weight of the coating and the ion-exchange matrix drug complex.

15. The composition of claim 1 or 3, wherein the dispersion medium comprises a highly hydrated excipient.

16. The composition of claim 15, wherein the dispersion medium comprises 50% to 70% on a weight by weight basis of a highly hydrated excipient.

17. The composition of claim 16, wherein the highly hydrated excipient is sucrose.

18. The composition of claim 1 or 3, further comprising a dispersion additive selected from the group consisting of a stabilizing agent, a dispersion agent, and any combination thereof.

19. The composition of claim 1 or 3, wherein one or more of the drugs in the dispersed phase or the dispersion medium are selected from a cardiovascular drug, respiratory drug, sympathomimetic drug, cholinomemetic drug, adrenergic drug, antimuscarinic drug, antispasmodic drug, skeletal muscle relaxant, diuretic drug, anti-migraine drug, anesthetic, sedative, hypnotic, antiepileptic, psychopharmacologic agent, analgesic, including opioid and non-opioid analgesic, antipyretic, CNS stimulant, antineoplastic, immunosuppressive drug, antimicrobial drug, antihistamine, anti-inflammatory, antibiotic, decongestant, cough suppressant, expectorant or a combination thereof.

20. The composition of claims 1, wherein the two or more drugs in the dispersed phase comprise an antihistamine, an antitussive, and optionally a decongestant.

21. The composition of claim 3, wherein the drugs in the dispersed phase or dispersion medium comprise an antihistamine, an antitussive, and optionally a decongestant.

22. The composition of claim 3, 6, or 21, wherein
administration of a single dose of the drug composition to a patient provides serum levels of the drugs of the dispersed phase and the dispersion medium over a time period of at least 8 hours that are bioequivalent to serum levels achieved upon administration of an appropriate number of doses, over the same time period, of FDA-approved immediate release reference listed drug (IR RLD) compositions comprised of said drugs, and wherein
the appropriate number of doses corresponds to a number of doses recommended in one or more FDA-approved labels for administration of the IR RLD compositions over the same time period.

23. A method for preparing a liquid form controlled release drug composition, comprising:
(a) preparing a dispersed phase, which comprises preparing particulates, pellets or beads comprising two or more drugs and a pharmaceutically acceptable ion-exchange matrix, wherein the two or more drugs bind to the ion-exchange matrix in a single particulate, pellet or bead;
(b) preparing a dispersion medium;
(c) coating the particulates, pellets or beads with a membrane coating; and
(d) dispersing the particulates, pellets or beads into the dispersion medium.

24. A method for preparing a liquid form controlled release drug composition, comprising:
(a) preparing a dispersed phase, which comprises preparing particulates, pellets or beads comprising one or more drugs associated with a pharmaceutically acceptable ion-exchange matrix;
(b) preparing a dispersion medium, wherein the dispersion medium comprises one or more drugs;
(c) coating the particulates, pellets or beads with a membrane coating; and
(d) dispersing the particulates, pellets or beads into the dispersion medium.

25. A method of claim 23 or 24, wherein the step of preparing the dispersed phase further comprises blending of the drugs and ion exchange matrix powders.

26. A method of claim 25, wherein the step of preparing the dispersed phase further comprises wet granulation, extrusion and spheronization of the drugs and ion exchange matrix powders.

27. A method of claim 23 or 24, wherein pharmaceutically acceptable salt forms of the drugs and the ion exchange matrix are used.

28. A liquid form controlled release drug composition comprising a dispersion medium and a dispersed phase, wherein
the dispersion medium comprises an antihistamine, an antitussive, and optionally a decongestant as active ingredients in an immediate release form,
the dispersed phase comprises particulates, pellets, or beads that comprise the antihistamine, the antitussive, and the decongestant as three active ingredients in an extended release form, wherein
administration of a single dose of the drug composition to a patient provides serum levels of the three active ingredients over a time period of at least 8 hours that are bioequivalent to serum levels achieved upon administration of an appropriate number of doses, over the same time period, of FDA-approved immediate release reference listed drug (IR RLD) compositions comprised of the active ingredients, and wherein
the appropriate number of doses corresponds to a number of doses recommended in one or more FDA-approved labels for administration of the IR RLD compositions over the same time period.

29. The drug composition of claim 28, wherein the antitussive is a narcotic antitussive.

30. The drug composition of claim 28, wherein the dispersion medium does not comprise the decongestant.

31. The drug composition of claim 28, wherein the particulate, pellet or bead further comprises a coating.

32. The drug composition of claim 28, wherein the antihistamine, the antitussive and the decongestant in the dispersed phase associate in a single particulate, pellet or bead, wherein the particulate, pellet or bead further comprises a pharmaceutically acceptable ion-exchange matrix, and wherein the antihistamine, the antitussive and the decongestant associate with the ion-exchange matrix.

33. The drug composition of claim 28, further comprising an additive selected from the group consisting of a stabilizing agent, a dispersion agent, and any combination thereof.

34. A liquid form controlled release drug composition comprising a dispersion medium and a dispersed phase, wherein
the dispersion medium comprises an antihistamine, an antitussive, and optionally a decongestant, as active ingredients in an immediate release form,
the dispersed phase comprises a particulate, pellet or bead that comprises the antihistamine, the antitussive, and the decongestant as three active ingredients in an extended release form, wherein
administration of a sufficient number of doses of the drug composition to a patient to achieve steady-state serum levels of the three active ingredients over a time period of greater than 24 hours yields serum levels of the active ingredients that are bioequivalent to serum levels achieved upon administration of an appropriate number of doses, over the same time period, of one or more FDA-approved immediate release drug compositions comprised of the active ingredients, and wherein
the appropriate number of doses corresponds to a number of doses recommended in one or more FDA-approved labels for the administration of the one or more FDA-approved immediate release drug compositions over the same time period.

35. A method for treating cough, cold, flu or allergy symptoms in a human subject in need thereof, comprising the step of administering the liquid form controlled release drug composition of claim 28 to the subject.

36. The method of claim 35, which allows a one-a-day or twice-a-day dosing in humans.

37. A method of treating coughing, symptoms of coughing, nasal discharge, congestion or sneezing associated with a cold, flu or an allergy for a time period of at least 8 hours, comprising administering to a human subject in need thereof a single dose of the drug composition of claim 28 effective to treat coughing, symptoms of coughing, nasal discharge, congestion or sneezing associated with a cold or an allergy, for a time period of at least 8 hours.

38. A method for making the liquid oral extended release drug composition of claim 28, comprising
(a) preparing the dispersion medium, wherein the dispersion medium comprises an antihistamine, an antitussive, and optionally a decongestant as active ingredients;
(b) preparing the dispersed phase, which comprises preparing particulates, pellets or beads comprising an antihistamine, an antitussive, and a decongestant as three active ingredients;
(c) coating the particulates, pellets or beads with a membrane coating; and
(d) combining the dispersed phase with the dispersion medium.

39. The method of claim 38, wherein the step of preparing the dispersed phase further comprises preparing particulates, pellets or beads, wherein the antihistamine, the antitussive, and the decongestant associate in a single particulate, pellet or bead, and wherein the step further comprises associating the antihistamine, the antitussive, and the decongestant with a pharmaceutically acceptable ion-exchange matrix.

40. A method for achieving in a mammal serum levels of an antihistamine, an antitussive and a decongestant over a time period of at least 8 hours that are bioequivalent to serum levels achieved upon administration of an appropriate number of doses over the same time period of FDA-approved immediate release reference listed drug (IR RLD) compositions to the same mammal, wherein the method comprises:
administering to the mammal a liquid form controlled release drug composition comprising a dispersion medium and a dispersed phase, wherein the dispersion medium comprises the antihistamine, the antitussive and optionally the decongestant as active ingredients in an immediate release form, and wherein the dispersed phase comprises a particulate, pellet, or bead that comprises the antihistamine, antitussive and the decongestant as three active ingredients in an extended release farm, and thereby
achieving serum levels of the three active ingredients over a time period of at least 8 hours that are bioequivalent to serum levels achieved upon administration of an appropriate number of doses over the same time period of FDA-approved IR RLD compositions comprising the active ingredients, wherein the appropriate number of doses corresponds to a number of doses recommended in one or more FDA-approved labels for the administration of the IR RLD compositions over the same time period.

41. A method for achieving in a mammal steady-state serum levels of an antihistamine, an antitussive and a decongestant upon administration of a liquid form controlled release drug composition, wherein the serum levels are bioequivalent to serum levels achieved upon administration of one or more immediate release (IR) compositions comprising the antihistamine, the antitussive and/or the decongestant to the same mammal, wherein the method comprises:
administering to the mammal a liquid form controlled release drug composition comprising a dispersion medium and a dispersed phase, wherein the dispersion medium comprises the antihistamine, the antitussive and optionally the decongestant as active ingredients in an immediate release form, and wherein the dispersed phase comprises a particulate, pellet or bead that comprises the antihistamine, the antitussive and the decongestant as active ingredients in an extended release form, and
wherein administration of a sufficient number of doses of the liquid form controlled release drug composition to the mammal to achieve steady-state serum levels of the three active ingredients over a time period of greater than 24 hours yields serum levels of the active ingredients that are bioequivalent to serum levels achieved upon administration of an appropriate number of doses over the same time period of one or more FDA-approved immediate release (IR) drug compositions comprising the active ingredients,
wherein the appropriate number of doses of the one or more FDA-approved IR drug compositions corresponds to a number of doses recommended in one or more FDA-approved labels for the administration of the one or more FDA-approved IR drug compositions over the same time period, and
wherein the appropriate number of doses of the one or more FDA-approved IR drug compositions is greater than the sufficient number of doses of the oral ER drug composition.
